SEQUENCE LISTING (39 sequences)

SEQ ID NO: 1 - protein, 685 aa, Thermus thermophilus
MNHLGKTEVFLNRFALRPLNPEELRPWRLEVVLDPPPGREEVYPLLAQVARRAGGVTVRMGDGLASWSPPEVLVLEGTLARMGQTYAYRLYPKGRRPLDPKDPGERSVLSALARRLLQERLRRLEGVWVEGLAVYRREHARGPGWRVLGGAVLDLWVSDSGAFLLEVDPAYRILCEMSLEAWLAQGHPLPKRVRNAYDRRTWELLRLGEEDPKELPLPGGLSLLDYHASKGRLQGREGGRVAWVADPKDPRKPIPHLTGLLVPVLTLEDLHEEEGSLALSLPWEERRRRTREIASWIGRRLGLGTPEAVRAQAYRLSIPKLMGRRAVSKPADALRVGFYRAQETALALLRLDGAQGWPEFLRRALLRAFGASGASLRLHTLHAHPSQGLAFREALRKAKEEGVQAVLVLTPPMAWEDRNRLKALLLREGLPSQILNVPLREEERHRWENALLGLLAKAGLQVVALSGAYPAELAVGFDAGGRESFRFGGAACAVGGDGGHLLWTLPEAQAGERIPQEVVWDLLEETLWAFRRKAGRLPSRVLLLRDGRVPQDEFALALEALAREGIAYDLVSVRKSGGGRVYPVQGRLADGLYVPLEDKTFLLLTVHRDFRGTPRPLKLVHEAGDTPLEALAHQIFHLTRLYPASGFAFPRLPAPLHLADRLVKEVGRLGIRHLKEVDREKLFFV

SEQ ID NO: 2 - protein, 9 aa, Artificial Sequence
  Description of Artificial Sequence Synthetic peptide
RRRRRRRRR

SEQ ID NO: 3 - protein, 9 aa, Artificial Sequence
  Description of Artificial Sequence Synthetic peptide
EEEEEEEEE

SEQ ID NO: 4 - protein, 6 aa, Artificial Sequence
  Description of Artificial Sequence Synthetic 6xHis tag
HHHHHH

SEQ ID NO: 5 - RNA, 21 nt, Artificial Sequence
  Description of Artificial Sequence Synthetic oligonucleotide
ugagguaguagguuguauagu

SEQ ID NO: 6 - RNA, 30 nt, Artificial Sequence
  Description of Artificial Sequence Synthetic oligonucleotide
cacacacuauacaaccuacuaccucaaccu

SEQ ID NO: 7 - RNA, 21 nt, Artificial Sequence
  Description of Artificial Sequence Synthetic oligonucleotide
uagcuuaucagacugauguug

SEQ ID NO: 8 - DNA, 16 bp, Artificial Sequence
  Description of Artificial Sequence Synthetic oligonucleotide
tgaggtagtaggttgt

SEQ ID NO: 9 - RNA, 21 nt, Artificial Sequence
  Description of Artificial Sequence Synthetic oligonucleotide
acuauacaaccuacuaccuca

SEQ ID NO: 10 - RNA, 21 nt, Artificial Sequence
  Description of Artificial Sequence Synthetic oligonucleotide
ugauauguuggaucuaccuca

SEQ ID NO: 11 - RNA, 21 nt, Artificial Sequence
  Description of Artificial Sequence Synthetic oligonucleotide
ugauaacaaggaucuaccuca

SEQ ID NO: 12 - RNA, 21 nt, Artificial Sequence
  Description of Artificial Sequence Synthetic oligonucleotide
acuauacaaccaaaaaaaaaa

SEQ ID NO: 13 - DNA, 21 bp, Artificial Sequence
  Description of Artificial Sequence Synthetic oligonucleotide
actatacaacctactacctca

SEQ ID NO: 14 - DNA, 21 bp, Artificial Sequence
  Description of Artificial Sequence Synthetic oligonucleotide
tgatatgttggatctacctca

SEQ ID NO: 15 - DNA, 21 bp, Artificial Sequence
  Description of Artificial Sequence Synthetic oligonucleotide
tgataacaaggatctacctca

SEQ ID NO: 16 - RNA, 21 nt, Artificial Sequence
  Description of Artificial Sequence Synthetic oligonucleotide
caacaucagucugauaagcua

SEQ ID NO: 17 - DNA, 16 bp, Artificial Sequence
  Description of Artificial Sequence Synthetic oligonucleotide
acaacctactacctca

SEQ ID NO: 18 - DNA, 16 bp, Artificial Sequence
  Description of Artificial Sequence Synthetic oligonucleotide
tgttggatctacctca

SEQ ID NO: 19 - DNA, 16 bp, Artificial Sequence
  Description of Artificial Sequence Synthetic oligonucleotide
acaaggatctacctca

SEQ ID NO: 20 - RNA, 16 nt, Artificial Sequence
  Description of Artificial Sequence Synthetic oligonucleotide
acaaccuacuaccuca

SEQ ID NO: 21 - RNA, 16 nt, Artificial Sequence
  Description of Artificial Sequence Synthetic oligonucleotide
uguuggaucuaccuca

SEQ ID NO: 22 - RNA, 16 nt, Artificial Sequence
  Description of Artificial Sequence Synthetic oligonucleotide
acaaggaucuaccuca

SEQ ID NO: 23 - RNA, 21 nt, Artificial Sequence
  Description of Artificial Sequence Synthetic oligonucleotide
ugauauguuggaucuaccaga

SEQ ID NO: 24 - RNA, 21 nt, Artificial Sequence
  Description of Artificial Sequence Synthetic oligonucleotide
ugauauguuggaucuacgaca

SEQ ID NO: 25 - RNA, 21 nt, Artificial Sequence
  Description of Artificial Sequence Synthetic oligonucleotide
ugauauguuggaucuaaauca

SEQ ID NO: 26 - RNA, 21 nt, Artificial Sequence
  Description of Artificial Sequence Synthetic oligonucleotide
ugauauguuggaucuugcuca

SEQ ID NO: 27 - RNA, 21 nt, Artificial Sequence
  Description of Artificial Sequence Synthetic oligonucleotide
ugauauguuggaucauccuca

SEQ ID NO: 28 - RNA, 21 nt, Artificial Sequence
  Description of Artificial Sequence Synthetic oligonucleotide
ugauauguuggaugaaccuca

SEQ ID NO: 29 - RNA, 21 nt, Artificial Sequence
  Description of Artificial Sequence Synthetic oligonucleotide
ugagguaguagguuguagagu

SEQ ID NO: 30 - RNA, 21 nt, Artificial Sequence
  Description of Artificial Sequence Synthetic oligonucleotide
ugauauguuggaucuacgaca

SEQ ID NO: 31 - RNA, 21 nt, Artificial Sequence
  Description of Artificial Sequence Synthetic oligonucleotide
acuauacaaccuacuauauca

SEQ ID NO: 32 - DNA, 16 bp, Artificial Sequence
  Description of Artificial Sequence Synthetic oligonucleotide
tggtctccctatagtg

SEQ ID NO: 33 - DNA, 32 bp, Artificial Sequence
  Description of Artificial Sequence Synthetic oligonucleotide
cactatagggagaccacaacggtttccctcta

SEQ ID NO: 34 - DNA, 32 bp, Artificial Sequence
  Description of Artificial Sequence Synthetic oligonucleotide
tagagggaaaccgttgtggtctccctatagtg

SEQ ID NO: 35 - DNA, 16 bp, Artificial Sequence
  Description of Artificial Sequence Synthetic oligonucleotide
caacggtttccctcta

SEQ ID NO: 36 - DNA, 16 bp, Artificial Sequence
  Description of Artificial Sequence Synthetic oligonucleotide
gcagcagccaactcag

SEQ ID NO: 37 - DNA, 32 bp, Artificial Sequence
  Description of Artificial Sequence Synthetic oligonucleotide
ctgagttggctgctgccaccgctgagcaataa

SEQ ID NO: 38 - DNA, 32 bp, Artificial Sequence
  Description of Artificial Sequence Synthetic oligonucleotide
ttattgctcagcggtggcagcagccaactcag

SEQ ID NO: 39 - DNA, 16 bp, Artificial Sequence
  Description of Artificial Sequence Synthetic oligonucleotide
caccgctgagcaataa

(19) United States
(12) Patent Application Publication
(10) Pub. No.: US 2016/0289734 A1
(43) Pub. Date: Oct. 6, 2016

(54) METHODS OF USING OLIGONUCLEOTIDE-GUIDED ARGONAUTE PROTEINS

(71) Applicant: University of Massachusetts, Boston, MA (US)

(72) Inventors: Phillip David Zamore, Northborough, MA (US); Melissa Jeanne Moore, Chestnut Hill, MA (US); Samson Michael Jolly, Holden, MA (US); William Edward Salomon, Worcester, MA (US); Victor Serebrov, Shrewsbury, MA (US); Han Zhang, Zhengzhou (CN)

(21) Appl. No.: 15/089,243

(22) Filed: Apr. 1, 2016

Related U.S. Application Data
(60) Provisional application No. 62/142,759, filed on Apr. 3, 2015.

(51) Int. Cl. C12Q 1/68; G01N 33/53

(52) U.S. Cl. CPC C12Q 1/683; C12Q 1/6806; C12Q 1/6818; G01N 33/5308

(57) ABSTRACT

The invention relates to the use of Argonaute polypeptide:guide molecule complexes as fast and specific nucleic acid probes, as specific, nucleic acid-guided restriction enzymes for DNA and RNA substrates, and as a means to detect RNA-protein interactions, RNA detection, DNA detection, and RNA depletion. Using such Argonaute polypeptide:guide molecule complexes enables fast and specific detection, purification, and enzymatic activity.

### RELATED APPLICATIONS

This application claims priority to U.S. Provisional Patent Application No. 62/142,759, filed Apr. 3, 2015, the contents of which are incorporated herein by reference.

### STATEMENT REGARDING FEDERALLY SPONSORED RESEARCH

This invention was made with government support under grant number GM62862 awarded by the National Institutes of Health. The government has certain rights in the invention.

### TECHNICAL FIELD

The invention relates to the use of Argonaute polypeptide:guide molecule complexes as fast and specific nucleic acid probes, as nucleic acid-guided, restriction enzymes for DNA and RNA substrates, and as a means to detect RNA-protein interactions, RNA detection, and RNA depletion.

### BACKGROUND

Quantitative analysis of gene expression is a fundamentally important approach in modern biological sciences and medicine. Detection and quantification of messenger and other specific RNAs is among the most widely used research and diagnostic techniques. All available approaches for detection as well as isolation of endogenous RNAs rely on the use of specific probes. Oligonucleotide probes are synthetic molecules that can be designed to bind RNA targets with high degree of specificity, and to provide means for quantitative detection of binding, such us fluorescent or other types of readout. Although protein-based probes that recognize specific RNA sequences can be used (Bao et al., Annu. Rev. Biomed. Eng. 11, 25-47, 2009; Urbanek et al., RNA Biol. 11, 1083-1095, 2014), e.g., the Pumilio family of proteins (Yoshimura et al., ACS Chem. Biol. 7:999-1005, 2012), a separate protein construct has to be engineered, produced, and purified for each RNA target, making this approach impractical. The most available methods use short DNA or modified RNA oligonucleotides that recognize their complementary RNA targets via base pairing. When used in imaging techniques, e.g., RNA in situ hybridization (RNA-ISH), these oligonucleotide probes are typically conjugated to fluorescent dyes for detection. In order to increase their signal-to-noise ratio, probes can be designed to form a stable hairpin that brings the fluorescent dye close to a quencher; the probe becomes highly fluorescent upon hybridization with the RNA target (molecular beacons). Several patented and commercialized strategies are available that provide post-hybridization amplification of the signal (hybrid capture, branched DNA assay, padlock probes) and multiplexing (such as available from NanoString Technologies (Seattle, Wash.)).

However, the sensitivity and specificity that can be achieved by all these strategies are limited by the inherent properties of the oligonucleotide probe itself. To achieve sufficient specificity with complex samples (e.g., the entire genome or transcriptome) probes need to be at least 15-20 nucleotides in length. Unfortunately, probes in this length range from exceptionally stable duplexes with their intended targets. Moreover, many unintended targets with partial complementarity to the probes will hybridize with lower but nonetheless high stability, leading to high levels of non-specific recognition (Herschlag, Proc. Natl. Acad. Sci. USA 88, 6921-6925 1991). To discriminate the specific, fully complementary targets from non-specific, partially complementary targets, hybridization needs to be carried out at temperatures high enough to prevent hybridization of mismatched targets. An alternative strategy for increasing probes' specificity involves using chemical denaturant such as formamide and urea. The lack of specificity under physiological conditions greatly limits the use of oligonucleotide probes in living cells, or in any application where denaturation must be avoided. In addition, oligonucleotide probes tend to be rapidly sequestered inside the cell nuclei, which make them unsuitable for detection of RNA in the cytoplasm. Oligonucleotide probes are also prone to degradation by nucleases. Their stability can be increased by using chemical modifications, but this significantly increases costs, and many of the modifications are toxic to cells. Another major drawback of oligonucleotide is their slow kinetics of hybridization to complementary sequences. This precludes using oligonucleotide probes to study dynamic cellular processes.

The ability to study, isolate, manipulate, and detect RNAs is essential for determining gene functions, gene expression and/or RNA sub-cellular localization. Currently, the state of the art is focused on two methods (1) RNA pull-down/affinity purification: in which RNAs are labeled (e.g. biotin) and these exogenous RNAs are used as bait in pull-down assays with immunoblotting or mass spectrometry analysis to identify proteins bound to the RNA or (2) RNA binding proteins (RBPs): expressing RNA recognition motifs (RRMs) alone or fused to other proteins to engineer specificity for RNAs (Filipovska and Rockham, RNA Biology, 8:6, 978-983, 2011). Such RBPs or their domains that have been adapted for such use include, Pumilio and FBF proteins (PUF), heterogeneous nuclear ribonucleoprotein K homology domains (KH), bacteriophage MS2 coat proteins (three hybrid systems), pentatricopeptide repeat (PPR) proteins, RNA binding Zinc finger domains and Cas9/sgRNA (MacKay et al., NSMB, 18(3), 256-261, 2011; O'Connel et al., Nature, 6(7530), 263-266, 2014; Wang et al., 2013; Zamore et al., Biochemistry, (38), 596-604, 1999). The first method, using RNA as bait, does not allow for study of endogenous RNAs and is susceptible to endogenous nucleases. The second method, using RNA-binding proteins, has varying degrees of programmability for specificity to RNA, but the examples of this method are designed for eukaryotic systems, require recombinant technology, genetic manipulation or protein purification for each unique RNA to be studied, or have moderate affinity for RNA. The need for a simple and general way to generate programmable protein complexes with specificities for endogenous RNAs of interest that is amenable for use in all three domains of life would represent a powerful tool and an advance within many fields of study.

Modification of nucleic acid sequences is a common practice in molecular biology. Modern recombinant DNA technology was made possible by the pioneering discovery that bacterial restriction enzymes can be used to cut double-stranded DNA at specific sequences (Cohen et al., 1973. Construction of biologically functional bacterial plasmids in vitro. Proc. Natl. Acad. Sci. USA 70, 3240-324). By cleaving DNA molecules from different sources with appropriately chosen restriction enzymes, sequences—including whole genes—from one organism can be inserted into DNA—such as a bacterial plasmid—from another. However, restriction enzymes have been identified for only a minority of the possible 6 or 8 bp sequences. Consequently, a convenient restriction site is often not available to cut a DNA at the most optimal site. PCR-based strategies have been devised to circumvent this limitation of restriction enzyme-based molecular “cloning” (Gibson et al., 2009. Enzymatic assembly of DNA molecules up to several hundred kilobases. Nat Methods 6, 343-345), but PCR often introduces sequence errors and rearrangements, requiring extensive quality control assays to confirm that the intended recombinant sequence has been generated. Fragmentation, trimming, or site-specific cutting is used in many applications such as cloning, nucleic acid preparation, high-throughput sequencing, and genome engineering. Restriction enzymes are commonly used to cut a piece of DNA at a specific site but they are limited by the availability of an enzyme with the desired recognition sequence and cleavage site (reviewed, Pingoud, Wilson, & Wende, Nuc. Acids Res., 42:7489-7527, 2014). Moreover, the use of multiple enzymes at the same time is limited by their being active in a single buffer; many combinations of restriction enzymes require incompatible conditions for activity. Additionally, a single restriction enzyme will cut as many times as there are recognition sequences with no ability to make recognition sequence sites more complex. Finally, restriction enzymes use only double-stranded DNA as substrate.

A second method for the sequence-specific cutting of nucleic acids employs RNase H. RNase H was discovered in calf thymus; it degrades only the RNA component of an RNA/DNA duplex (Stein & Hausen, Science, 166:393-395, 1969). RNase H can be used to cut an RNA molecule at a specific sequence by supplying a DNA oligonucleotide in trans to direct the cut. The cleavage site of RNase H is imprecise, even when chemical modifications are introduced into the DNA guide to limit RNA cleavage to a small region of the total sequence paired to the oligonucleotide (Cerritelli & Crouch, FEBS Lett., 276:1494-1505, 2009). Chemically modified antisense oligonucleotides have also been developed as drugs for human diseases. Such antisense oligonucleotides act to recruit RNase H to specific mRNAs in vivo (reviewed Crooke ST (ed) (2008) Antisense drug technology: principles, strategies, and applications, 2nd edn. CRC, Boca Raton). Thermostable versions of RNase H are sold commercially for research use (see U.S. Pat. No. 5,268,289).

### SUMMARY OF INVENTION

In a first aspect, disclosed herein is a method of cleaving an RNA or DNA molecule, comprising binding to a target RNA or DNA sequence a complex comprising an Argonaute polypeptide and a heterologous, single-stranded oligonucleotide guide molecule that comprises a recruiting domain comprising at least 8 nucleotides at the 5′ end of the guide molecule and a stabilization domain adjacent and 3′ to the recruiting domain and comprising at least 4 nucleotides in a sample, wherein the stabilization domain of the guide molecule has sufficient complementarity to its target RNA or DNA sequence such that the Argonaute polypeptide:guide molecule complex binds stably to the target RNA or DNA sequence, and allowing the Argonaute polypeptide:guide molecule to cleave the RNA or DNA molecule. In an embodiment, the stabilization domain consists of 4-8 nucleotides, such as 4, 5, 6, 7, and 8 nucleotides. In yet other embodiments, the recruiting domain consists of 8 nucleotides, and the stabilization domain consists of 4-8 nucleotides, such as 4, 5, 6, 7, and 8 nucleotides.

In other embodiments, the oligonucleotide guide molecule is a DNA guide molecule. In further embodiments, the target RNA or DNA molecule is cleaved at a phosphodiester bond across from nucleotide position 10 and 11 of the guide strand. In embodiments, the target RNA or DNA is single-stranded or double-stranded. In embodiments, binding of the Argonaute polypeptide:guide molecule complex to the target RNA or DNA molecule is at least 10- to 300-times faster than the guide molecule binding the target alone. In embodiments, the Argonaute polypeptide:guide molecule complex binding to the target RNA or DNA molecule has a dissociation constant (KD)<1 nM. In other embodiments, the stabilization domain has about 38-100% complementarity to its target RNA or DNA sequence, such as about 50%, 63%, 75%, 88%, and about 100% complementarity to its target RNA or DNA sequence. In other embodiments, the guide molecule comprises one or more mismatches 3′ of g5. In embodiments, the guide molecule comprises two or more mismatches 3′ of g5; in further embodiments, the guide molecule comprises two mismatches 3′ of g5 and 5′ of g9. In other further embodiments, the guide molecule comprises two mismatches 3′ of g8 to the 3′ end of the molecule. In other embodiments, the guide molecule consists of 12-16 nucleotides, such as 12, 13, 14, 15, and 16 nucleotides.

In embodiments, the guide molecule comprises a nucleotide sugar modification or a nucleotide substitution. In some embodiments, the nucleotide sugar modification comprises a 2′ sugar modification and is selected from the group consisting of a 2′-O—CH3, a 2′-F, and a 2′-MOE modification. In other embodiments, the nucleotide substitution comprises one selected from the group consisting of locked nucleic acid (LNA), an unlocked nucleic acid (UNA), deoxyuridine, pseudouridine, 5-methylcytosine, 2-aminopurine, 2,6-diaminopurine, deoxyinosine, 5-hydroxybutynl-2′-deoxyuridine, 8-aza-7-deazaguanosine, and 5-nitroindole. In further embodiments, the guide molecule comprises a sugar modification and a nucleotide substitution.

In embodiments, the Argonaute polypeptide is a Thermus thermophilus Argonaute polypeptide. In embodiments, the target molecule is RNA and cleavage is accomplished by incubating the sample at about 55° C. or greater. In yet other embodiments, the target molecule is DNA and cleavage is accomplished by incubating the sample at about 65° C. or greater. In embodiments, the sample comprises a solution comprising a salt, such as KCl, NaCl, or C₅H₈NNaO₄ (monosodium glutamate). In embodiments, the solution comprises about 25 mM to about 100 mM. In other embodiments, the solution comprises about 50 mM NaCl, about 75 mM C₅H₈NNaO₄ (monosodium glutamate), or about 100 mM KCl. The solution can have a pH of about 7 to about 8.8, such as 7.4 to 7.5. The solution can further comprise a buffer. In embodiments, the buffer is selected from the group consisting of N-(2-acetamido)-2-aminoethanesulfonic acid (ACES), N-(2-acetamido)iminodiacetic acid (ADA), N,N-bis(2-hydroxyethyl)-2-aminoethanesulfonic acid (BES), 2-(N-morpholino)ethanesulfonic acid (MES), 3-(N-morpholino)-propanesulfonic acid (MOPS), 3-(N-morpholinyl)-2-hydroxypropanesulfonic acid (MOPSO), piperazine-N,N′-bis(2-ethanesulfonic acid) [Pipes], N-tris-(hyrdroxymethyl)-methyl-2-aminoethanesulfonic acid (TES), 3-[N-tris (hydroxymethyl) methylamino]-2-hydroxypropanesulfonic acid (TAPSO), and 3-[N-tris-(hydroxymethyl-mettlylamino]-propanesulfonic acid (TAPS); and 2-[4-(2-hydroxyethyl)piperazin-1-yl]ethanesulfonic acid (HEPES). In some embodiments, the buffer comprises 2-[4-(2-hydroxyethyl)piperazin-1-yl]ethanesulfonic acid (HEPES) or HEPES-KOH. The concentration of the buffer can be from about 1 mM to about 200 mM; in other embodiments, the concentration can be about 18 mM. In embodiments, the solution further comprises a reducing agent, such as dithiothreitol (DTT) or 2-mercaptoethanol (β-mercaptoethanol); and can be present at about 1 mM to about 20 mM; in some embodiments, the concentration is 5 mM. In yet other embodiments, the solution can further comprise a detergent, such as a nonionic, non-denaturing detergent or a zwitterionic nondenaturing detergent. In some embodiments, the detergent is selected from the group consisting of poly(ethyleneoxy)ethanol (IGEPAL®-CA630, Nonidet™ P-40); Octylphenolpoly(ethyleneglycolether)×(Triton® X-100), Polyethylene glycol tert-octylphenyl ether (Triton® X-114), Polyoxyethylene (23) lauryl ether (Brij® 35), Polyethylene glycol hexadecyl ether (Brij® 58), Polyethylene glycol sorbitan monolaurate (Tween® 20), Polyethylene glycol sorbitan monooleate (Tween® 80), and octylglucoside. In a preferred embodiment, the detergent is poly(ethyleneoxy)ethanol. Examples of zwitterionic nondenaturing detergents include 3-((3-cholamidopropyl) dimethylammonio)-1-propanesulfonate (CHAPS) and 3-([3-Cholamidopropyl]dimethylammonio)-2-hydroxy-1-propanesulfonate (CHAPSO). In some embodiments, the detergent comprises octylphenoxy poly(ethyleneoxy)ethanol, branched (IGEPAL®-CA630, Nonidet™ P-40). The detergent can be present at about 0.001% to about 2%; in some embodiments, the detergent is present at about 0.01%. The solution can further comprise glycerol or a sugar (such as sucrose), and can be present at about 1% to about 20%; in some embodiments, the glycerol or sugar is present at about 10%. In some embodiments, the solution comprises 18 mM HEPES-KOH, pH 7.4; 50 mM NaCl, 3 mM MnCl₂, 0.01% octylphenoxy poly(ethyleneoxy)ethanol, 5 mM DTT, and 10% glycerol. In other embodiments, the solution comprises 18 mM HEPES-KOH, pH 7.4; 75 mM C₅H₈NNaO₄, 3 mM MnCl₂, 0.01% octylphenoxy poly(ethyleneoxy)ethanol, 5 mM DTT, and 10% glycerol. In yet other embodiments, the solution comprises 18 mM HEPES-KOH, pH 7.4; 100 mM KCl, 3 mM MnCl₂, 0.01% octylphenoxy poly(ethyleneoxy)ethanol, 5 mM DTT, and 10% glycerol. In embodiments, the solution further comprises a divalent metal cation, such as Mn²⁺ or Mg²⁺. In embodiments, the divalent metal cation is present as a salt from about 1 mM to about 100 mM; in some embodiments, the salt of a divalent metal cation is present at about 3 mM. In further embodiments of these solutions, the guide molecule can consist of 12 to 15 nucleotides, such as 12, 13, 14, and 15 nucleotides. In embodiments, the Argonaute polypeptide:guide molecule specifically cleaves at its target sequence. The cleavage can occur in vitro. In embodiments, the Argonaute polypeptide:guide molecule complex specifically cleaves at its target sequence.

In some embodiments, the Argonaute polypeptide:guide molecule complex is attached to a solid support. The solid support can comprise at least one selected from the group consisting of agarose, cross-linked agarose, cellulose, dextran, polyacrylamide, latex, polystyrene, polyethylene, polypropylene, polyfluoroethylene, polyethyleneoxy, glass, silica, controlled pore glass, reverse phase silica, and metal. In further embodiments, the Argonaute polypeptide comprises an affinity tag and is attached to the solid support by the affinity tag binding to a binding partner, wherein the binding partner is immobilized on the solid support. The affinity tag can be, for example, biotin, and the binding partner is avidin or streptavidin.

In some embodiments, the sample comprises a cell, such as a prokaryotic or eukaryotic cell. The cell can be alive. In other embodiments, the sample comprises a cell extract or a bodily fluid. In yet other embodiments, the sample comprises purified RNA or DNA. In other embodiments, the sample can comprise a plasmid. In yet other embodiments, the sample comprises in vitro transcribed mRNA.

In some embodiments, the Argonaute polypeptide comprises an additional polypeptide sequence. The additional polypeptide sequence can comprise a sequence selected from the group consisting of a nuclear localization sequence, a mitochondrial localization sequence, and a chloroplast localization sequence. In some embodiments, the target is an RNA molecule, and the RNA molecule is selected from the group consisting of a nuclear, a mitochondrial, a plastid, and a viral RNA molecule. In other embodiments, the target is a DNA molecule, the DNA molecule is selected from the group consisting of a nuclear, a mitochondrial, a plastid, and a viral DNA molecule.

An embodiment is directed to a method of subcloning a desired double stranded nucleic acid fragment from a donor double stranded nucleic acid molecule (donor fragment) to an acceptor double stranded nucleic acid molecule (acceptor molecule), comprising the steps of:

(a) cleaving the desired donor fragment according to the method of the first aspect, wherein
(i) a first Argonaute:guide molecule complex targets a first region of a first strand of the donor fragment;
(ii) a second Argonaute guide molecule complex that targets the first region of a second strand of the donor fragment, such that the targeted region of the first strand and the targeted region of the second strand partially overlap such that cleavage by the first and second Argonaute:guide molecule complex creates a first sticky end;
(iii) a third Argonaute:guide molecule complex that targets a second region of the first strand of the donor fragment;
(iv) a fourth Argonaute:guide molecule complex targets the second region of the second strand of the donor fragment as that of the third Argonaute:guide molecule complex, such that the targeted region of the first strand of the target nucleic acid and the targeted region of the second strand of target nucleic acid partially overlap such that cleavage by the third and fourth Argonaute:guide molecule complex creates a second sticky end;
(v) isolating the cleaved desired fragment;

(b) cleaving the acceptor molecule according to the method of the first aspect, wherein
(i) steps (a)(i)-(a)(iv) are repeated for the acceptor molecule, thus creating third and fourth sticky ends that are complementary to the first and second sticky ends;
(ii) isolating the cleaved acceptor molecule; and

(c) combining the molecules from steps (a) and (b) to create a mixture and incubating the mixture under appropriate conditions to form a new molecule comprising the desired donor fragment subcloned into the acceptor molecule. In some embodiments, ligase is added to the mixture of step (c). In other embodiments, the sticky ends are from about 18 to 24 nucleotides long, and ligase is not added to the mixture of step (c). In yet other embodiments, the sticky ends are not complementary, and the method further comprises in step (c) combining a first single-stranded oligonucleotide that is complementary to a sticky end of the desired fragment and to a sticky end of the acceptor molecule such that the oligonucleotide bridges the sticky ends, and a second single-stranded oligonucleotide that is complementary to the other sticky ends of the desired fragment and of the acceptor molecule, such that the oligonucleotide bridges the sticky ends; and treating the mixture with polymerase and ligase.

In a second aspect, disclosed herein is a kit comprising an Argonaute polypeptide and a single-stranded oligonucleotide guide molecule that comprises a recruiting domain comprising 8 nucleotides at the 5′ end of the guide molecule and a stabilization domain adjacent and 3′ to the recruiting domain and comprising at least 4 nucleotides and having a sequence sufficiently complementary to a target RNA or DNA molecule nucleic acid sequence such that the Argonaute polypeptide:guide molecule complex binds stably to the target RNA or DNA sequence. In an embodiment, the guide molecule is a DNA guide molecule. The kit can also comprise a buffer suitable for the Argonaut polypeptide and guide molecule to form a complex. In other embodiments, the buffer is suitable for the Argonaute polypeptide:guide molecule complex to cleave the target RNA or DNA molecule. The buffer can comprise at least one selected from the group consisting of: a buffer, a salt, a detergent, a reducing agent, a divalent metal cation, glycerol and sugar. The buffer can be selected from the group consisting of N-(2-acetamido)-2-aminoethanesulfonic acid (ACES), N-(2-acetamido)iminodiacetic acid (ADA), N,N-bis(2-hydroxyethyl)-2-aminoethanesulfonic acid (BES), 2-(N-morpholino)ethanesulfonic acid (MES), 3-(N-morpholino)-propanesulfonic acid (MOPS), 3-(N-morpholinyl)-2-hydroxypropanesulfonic acid (MOPSO), piperazine-N,N′-bis(2-ethanesulfonic acid) [Pipes], N-tris-(hyrdroxymethyl)-methyl-2-aminoethanesulfonic acid (TES), 3-[N-tris (hydroxymethyl) methylamino]-2-hydroxypropanesulfonic acid (TAPSO), and 3-[N-tris-(hydroxymethyl-mettlylamino]-propanesulfonic acid (TAPS); and 2-[4-(2-hydroxyethyl)piperazin-1-yl]ethanesulfonic acid (HEPES). In embodiments, the salt is NaCl, KCl, or C₅H₈NNaO₄. The detergent can be a nonionic non-denaturing detergent or a nondenaturing zwitterionic detergent. The divalent cation is Mg²⁺ or Mn²⁺. In some embodiments, the buffer can comprise either (1) 18 mM HEPES-KOH, pH 7.4; 50 mM NaCl, 3 mM MnCl₂, 0.01% octylphenoxy poly(ethyleneoxy)ethanol, 5 mM DTT, and 10% glycerol, (2) 18 mM HEPES-KOH, pH 7.4; 75 mM C₅H₈NNaO₄, 3 mM MnCl₂, 0.01% octylphenoxy poly(ethyleneoxy)ethanol, 5 mM DTT, and 10% glycerol, or (3) 18 mM HEPES-KOH, pH 7.4; 100 mM KCl, 3 mM MnCl₂, 0.01% octylphenoxy poly(ethyleneoxy)ethanol, 5 mM DTT, and 10% glycerol. The buffer can be prepared concentrated from about two-fold to about five-fold.

In some embodiments, the stabilization domain has about 38-100% complementarity to its target RNA or DNA sequence, such as about 50%, 63%, 75%, 88%, and about 100% complementarity to its target RNA or DNA sequence. In other embodiments, the guide molecule comprises one or more mismatches 3′ of g5. In embodiments, the guide molecule comprises two or more mismatches 3′ of g5; in further embodiments, the guide molecule comprises two mismatches 3′ of g5 and 5′ of g9. In other further embodiments, the guide molecule comprises two mismatches 3′ of g8 to the 3′ end of the molecule. In other embodiments, the guide molecule consists of 12-16 nucleotides, such as 12, 13, 14, 15, and 16 nucleotides.

In an embodiment, the guide molecule binds a disease marker sequence, a disorder marker sequence, or an infectious agent sequence.

In some embodiments, the guide molecule further comprises a detectable label, such as a fluorescent dye or a radiolabel. The detectable label can be at the 3′ end of the guide molecule.

In other embodiments, the detectable label is at least one fluorophore, the fluorophore localized to the recruiting or stabilization domain forming a first arm, wherein the guide molecule comprises additional sequence at the 3′ end that is complementary to the domain comprising the at least one fluorophore, the sequence labeled with at least one quencher and forming a second arm; the first arm separated from the second arm by not more than about 60 nucleotides; the guide molecule forming with the target RNA or DNA sequence under detection conditions a double-stranded hybrid having a first strength; the first and second arm sequences having sufficient complementarity to one another to form under detection conditions a double-stranded stem hybrid having a second strength less than the first strength, whereby in the absence of the target RNA or DNA sequence fluorescence of the at least one fluorophore is quenched; and wherein the first and second hybrid strengths being selected such that the guide molecule fluoresces when the at least one fluorophore is stimulated under detection conditions in the presence of the target RNA or DNA sequence.

In some embodiments, the guide molecule comprises a nucleotide sugar modification or a nucleotide substitution. In some embodiments, the nucleotide sugar modification comprises a 2′ sugar modification and is selected from the group consisting of a 2′-O—CH3, a 2′-F, and a 2′-MOE modification. In other embodiments, the nucleotide substitution comprises one selected from the group consisting of locked nucleic acid (LNA), an unlocked nucleic acid (UNA), deoxyuridine, pseudouridine, 5-methylcytosine, 2-aminopurine, 2,6-diaminopurine, deoxyinosine, 5-hydroxybutynl-2′-deoxyuridine, 8-aza-7-deazaguanosine, and 5-nitroindole. In further embodiments, the guide molecule comprises a sugar modification and a nucleotide substitution.

In some embodiments, the kit further comprises a probe to detect the target RNA or DNA sequence.

In embodiments, the guide molecule further comprises an additional sequence added 3′ of the guide molecule. In other embodiments, the Argonaute polypeptide comprises an affinity tag, such as biotin. The kit can further comprise an avidin or streptavidin component, such as a solid support comprising the avidin or streptavidin.

In an embodiment, the Argonaute polypeptide is altered. Such alteration can comprise an additional polypeptide sequence; or such altering can comprise altering a catalytic domain of the Argonaute polypeptide. An additional polypeptide sequence can comprise one selected from the group consisting of a nuclear localization sequence, a mitochondrial localization sequence, and a chloroplast localization sequence.

In some embodiments, the Argonaute polypeptide is a Thermus thermophilus Argonaute polypeptide.

In other embodiments, the Argonaute polypeptide and guide molecule are provided as a complex. The complex can be attached to a solid support, selected from the group consisting of agarose, cross-linked agarose, cellulose, dextran, polyacrylamide, latex, polystyrene, polyethylene, polypropylene, polyfluoroethylene, polyethyleneoxy, glass, silica, controlled pore glass, reverse phase silica, and metal.

In embodiments, the kit further comprises instructions for use.

In a third aspect, provided herein is a method of recruiting an Argonaute polypeptide to a heterologous target RNA or DNA sequence comprising combining the Argonaute polypeptide with a heterologous, single-stranded oligonucleotide guide molecule that comprises a recruiting domain of at least 8 nucleotides at its 5′ end and a stabilization domain adjacent and 3′ to the recruiting domain and comprising at least 4 nucleotides, wherein the stabilization domain of the guide molecule has sufficient complementarity to the target RNA or DNA sequence such that the Argonaute polypeptide:guide molecule complex stably binds to the target RNA or DNA sequence. In an embodiment, the stabilization domain consists of 4-8 nucleotides, such as 4, 5, 6, 7, and 8 nucleotides. In yet other embodiments, the recruiting domain consists of 8 nucleotides, and the stabilization domain consists of 4-8 nucleotides, such as 4, 5, 6, 7, and 8 nucleotides.

In other embodiments, the oligonucleotide guide molecule is a DNA guide molecule. In embodiments, the target RNA or DNA is single-stranded or double-stranded. In embodiments, binding of the Argonaute polypeptide:guide molecule complex to the target RNA or DNA molecule is at least 10- to 300-times faster than the guide molecule binding the target alone. In embodiments, the Argonaute polypeptide:guide molecule complex binding to the target RNA or DNA molecule has a dissociation constant (KD)<1 nM. In other embodiments, the stabilization domain has about 38-100% complementarity to its target RNA or DNA sequence, such as about 50%, 63%, 75%, 88%, and about 100% complementarity to its target RNA or DNA sequence. In other embodiments, the guide molecule comprises one or more mismatches 3′ of g5. In embodiments, the guide molecule comprises two or more mismatches 3′ of g5; in further embodiments, the guide molecule comprises two mismatches 3′ of g5 and 5′ of g9. In other further embodiments, the guide molecule comprises two mismatches 3′ of g8 to the 3′ end of the molecule. In other embodiments, the guide molecule consists of 12-16 nucleotides, such as 12, 13, 14, 15, and 16 nucleotides.

In some embodiments, the guide molecule comprises a nucleotide sugar modification or a nucleotide substitution. In some embodiments, the nucleotide sugar modification comprises a 2′ sugar modification and is selected from the group consisting of a 2′-O—CH3, a 2′-F, and a 2′-MOE modification. In other embodiments, the nucleotide substitution comprises one selected from the group consisting of locked nucleic acid (LNA), an unlocked nucleic acid (UNA), deoxyuridine, pseudouridine, 5-methylcytosine, 2-aminopurine, 2,6-diaminopurine, deoxyinosine, 5-hydroxybutynl-2′-deoxyuridine, 8-aza-7-deazaguanosine, and 5-nitroindole. In further embodiments, the guide molecule comprises a sugar modification and a nucleotide substitution.

In embodiments, the Argonaute polypeptide is a prokaryotic Argonaute polypeptide, for example a Thermus thermophilus Argonaute polypeptide.

In embodiments, the heterologous target RNA or DNA is a eukaryotic, prokaryotic, or viral mRNA or gene. In other embodiments, the heterologous guide molecule is chemically modified with one or more modified nucleotides.

In some embodiments, the Argonaute polypeptide:guide molecule complex is attached to a solid support. The solid support can comprise at least one selected from the group consisting of agarose, cross-linked agarose, cellulose, dextran, polyacrylamide, latex, polystyrene, polyethylene, polypropylene, polyfluoroethylene, polyethyleneoxy, glass, silica, controlled pore glass, reverse phase silica, and metal. In further embodiments, the Argonaute polypeptide comprises an affinity tag and is attached to the solid support by the affinity tag binding to a binding partner, wherein the binding partner is immobilized on the solid support. The affinity tag can be, for example, biotin, and the binding partner is avidin or streptavidin.

In some embodiments, the sample comprises a cell, such as a prokaryotic or eukaryotic cell. The cell can be alive. In other embodiments, the sample comprises a cell extract or a bodily fluid. In yet other embodiments, the sample comprises purified RNA or DNA. In other embodiments, the sample can comprise a plasmid. In yet other embodiments, the sample comprises in vitro transcribed mRNA.

In some embodiments, the Argonaute polypeptide comprises an additional polypeptide sequence. The additional polypeptide sequence can comprise a sequence selected from the group consisting of a nuclear localization sequence, a mitochondrial localization sequence, and a chloroplast localization sequence. In some embodiments, the target is an RNA molecule, and the RNA molecule is selected from the group consisting of a nuclear, a mitochondrial, a plastid, and a viral RNA molecule. In other embodiments, the target is a DNA molecule, the DNA molecule is selected from the group consisting of a nuclear, a mitochondrial, a plastid, and a viral DNA molecule.

In embodiments, contacting the Argonaute polypeptide:guide molecule complex to a sample depletes the sample of the target RNA or DNA molecule, such as an rRNA sequence. In other embodiments, contacting the Argonaute polypeptide:guide molecule complex to a sample isolates the target RNA or DNA molecule. In other embodiments, the target comprises an RNA molecule, and the guide molecule targets a splice site on the mRNA molecule. In further embodiments, the Argonaute polypeptide:guide molecule complex inhibits mRNA splicing at the splice site at least partially. Such inhibiting mRNA splicing can be in a living cell or in vitro.

In some embodiments, the method further comprises detecting the Argonaute polypeptide:guide molecule complex. Thus, in some embodiments, the guide molecule further comprises a detectable label, which can be a fluorescent dye or a radiolabel. In some embodiments, the detectable label is a fluorescent dye that is at the 3′ end of the guide molecule. In other embodiments, the detecting comprises detecting the target RNA or DNA nucleotide sequence. In embodiments, detecting the target RNA or DNA nucleotide sequence comprises using a probe to detect the target RNA or DNA nucleotide sequence.

In other embodiments, the detectable label is at least one fluorophore, the fluorophore localized to the recruiting or stabilization domain forming a first arm, wherein the guide molecule comprises additional sequence at the 3′ end that is complementary to the domain comprising the at least one fluorophore, the sequence labeled with at least one quencher and forming a second arm; the first arm separated from the second arm by not more than about 60 nucleotides; the guide molecule forming with the target RNA or DNA sequence under detection conditions a double-stranded hybrid having a first strength; the first and second arm sequences having sufficient complementarity to one another to form under detection conditions a double-stranded stem hybrid having a second strength less than the first strength, whereby in the absence of the target RNA or DNA sequence fluorescence of the at least one fluorophore is quenched; and wherein the first and second hybrid strengths being selected such that the guide molecule fluoresces when the at least one fluorophore is stimulated under detection conditions in the presence of the target RNA or DNA sequence. In some embodiments, the guide molecule further comprises an additional sequence added to the 3′ end of the guide molecule; the detecting can be detecting the additional sequence of the guide molecule. The detecting of the Argonaute polypeptide:guide molecule complex can be in a cell, such as a prokaryotic or eukaryotic cell; the cell can be alive. In other embodiments, the detecting of the Argonaute polypeptide:guide molecule complex is in a cell extract or a bodily fluid.

In some embodiments, the target RNA or DNA molecule encodes a disease marker sequence, a disorder marker sequence, or an infectious agent sequence.

In yet other embodiments, the Argonaute polypeptide can be altered, for example, by comprising an additional polypeptide sequence. Altering the Argonaute polypeptide can also comprise altering a catalytic domain of the Argonaute polypeptide, for example, by removing nucleic acid cleaving activity. An additional polypeptide sequence can comprise a sequence selected from the group consisting of a nuclear localization sequence, a mitochondrial localization sequence, and a chloroplast localization sequence.

In some embodiments, the target is an RNA molecule, the RNA molecule is selected from the group consisting of a nuclear, a mitochondrial, a plastid, and a viral RNA molecule. In other embodiments, the target is a DNA molecule, the DNA molecule is selected from the group consisting of a nuclear, a mitochondrial, a plastid, and a viral DNA molecule.

In a fourth aspect, disclosed herein is a method of identifying an RNA binding polypeptide comprising binding to a target RNA sequence in an RNA molecule a complex comprising an Argonaute polypeptide and a heterologous, single-stranded oligonucleotide guide molecule that comprises a recruiting domain comprising at least 8 nucleotides at its 5′ end and a stabilization domain adjacent and 3′ to the recruiting domain and comprising at least 4 nucleotides in a sample, wherein the stabilization domain of the guide molecule has sufficient complementarity to the target RNA sequence such that the Argonaute polypeptide:guide molecule complex binds stably to the target RNA sequence, isolating the Argonaute polypeptide:guide molecule complex bound to the target RNA sequence, and detecting polypeptides bound to the RNA molecule comprising the target RNA sequence. In an embodiment, the stabilization domain consists of 4-8 nucleotides, such as 4, 5, 6, 7, and 8 nucleotides. In yet other embodiments, the recruiting domain consists of 8 nucleotides, and the stabilization domain consists of 4-8 nucleotides, such as 4, 5, 6, 7, and 8 nucleotides.

In embodiments, the oligonucleotide guide molecule is a DNA guide molecule. In embodiments, the target RNA is single-stranded or double-stranded. In embodiments, binding of the Argonaute polypeptide:guide molecule complex to the target RNA molecule is at least 10- to 300-times faster than the guide molecule binding the target alone. In embodiments, the Argonaute polypeptide:guide molecule complex binding to the target RNA molecule has a dissociation constant (KD)<1 nM. In other embodiments, the stabilization domain has about 38-100% complementarity to its target RNA sequence, such as about 50%, 63%, 75%, 88%, and about 100% complementarity to its target RNA sequence. In other embodiments, the guide molecule comprises one or more mismatches 3′ of g5. In embodiments, the guide molecule comprises two or more mismatches 3′ of g5; in further embodiments, the guide molecule comprises two mismatches 3′ of g5 and 5′ of g9. In other further embodiments, the guide molecule comprises two mismatches 3′ of g8 to the 3′ end of the molecule. In other embodiments, the guide molecule consists of 12-16 nucleotides, such as 12, 13, 14, 15, and 16 nucleotides.

In some embodiments, the guide molecule comprises a nucleotide sugar modification or a nucleotide substitution. In some embodiments, the nucleotide sugar modification comprises a 2′ sugar modification and is selected from the group consisting of a 2′-O—CH3, a 2′-F, and a 2′-MOE modification. In other embodiments, the nucleotide substitution comprises one selected from the group consisting of locked nucleic acid (LNA), an unlocked nucleic acid (UNA), deoxyuridine, pseudouridine, 5-methylcytosine, 2-aminopurine, 2,6-diaminopurine, deoxyinosine, 5-hydroxybutynl-2′-deoxyuridine, 8-aza-7-deazaguanosine, and 5-nitroindole. In further embodiments, the guide molecule comprises a sugar modification and a nucleotide substitution.

In embodiments, the Argonaute polypeptide is a prokaryotic Argonaute polypeptide, for example a Thermus thermophilus Argonaute polypeptide.

In embodiments, the heterologous target RNA is a eukaryotic, prokaryotic, or viral mRNA or gene. In other embodiments, the heterologous guide molecule is chemically modified with one or more modified nucleotides.

In some embodiments, the Argonaute polypeptide:guide molecule complex is attached to a solid support. The solid support can comprise at least one selected from the group consisting of agarose, cross-linked agarose, cellulose, dextran, polyacrylamide, latex, polystyrene, polyethylene, polypropylene, polyfluoroethylene, polyethyleneoxy, glass, silica, controlled pore glass, reverse phase silica, and metal. In further embodiments, the Argonaute polypeptide comprises an affinity tag and is attached to the solid support by the affinity tag binding to a binding partner, wherein the binding partner is immobilized on the solid support. The affinity tag can be, for example, biotin, and the binding partner is avidin or streptavidin.

In some embodiments, the sample comprises a cell, such as a prokaryotic or eukaryotic cell. The cell can be alive. In other embodiments, the sample comprises a cell extract or a bodily fluid. In yet other embodiments, the sample comprises purified RNA. In other embodiments, the sample can comprise a plasmid. In yet other embodiments, the sample comprises in vitro transcribed mRNA.

In some embodiments, the Argonaute polypeptide is altered, such as by comprising an additional polypeptide sequence. The altering of the Argonaute polypeptide can comprise altering a catalytic domain of the Argonaute polypeptide, such as removing nucleic acid cleaving activity. The additional polypeptide sequence can comprise a sequence selected from the group consisting of a nuclear localization sequence, a mitochondrial localization sequence, and a chloroplast localization sequence.

In some embodiments, the target is an RNA molecule, the RNA molecule is selected from the group consisting of a nuclear, a mitochondrial, a plastid, and a viral RNA molecule.

### BRIEF DESCRIPTION OF THE DRAWINGS

FIG. 1 shows binding of a guide strand and Argonaute polypeptide according to the methods of the invention. (A) The guide strand having both a recruiting domain and a stabilization domain by itself slowly binds its target with low specificity. (B) However, in the presence of an Argonaute protein and the guide molecule having both domains binds quickly and with high specificity to its target. (C) shows the different parts involved in the targeting and binding of a guide strand and Argonaute polypeptide.

FIG. 2 shows various applications of the guide strand and Argonaute polypeptide complexes according to the methods of the invention. (A) shows isolation of a target sequence to which are bound proteins. In a first step, a lysate is prepared, then a complex of Argonaute:guide molecule (“Ago-guide”) that has been biotinylated (indicated by the letter “B”) is added to the lysate. The complex quickly and stably binds to its target. The complex bound to its target can be isolated using streptavidin-coated beads, and the proteins (open circles) analyzed by liquid chromatography-tandem mass spectrometry (LC-MS/MS). (B) shows an assay for determining the presence of a target. A substrate is prepared with complexes of Argonaute:guide molecule, immobilized by biotin (indicated by the letter “B”) to a streptavidin-coated surface. Samples are then applied, and the plate is probed for binding of the target to the Argonaute:guide molecule complex, visualized with a probe to the target sequence (filled circle). (C) shows a method of depleting a sample of a target nucleic acid. A sample is passed through a column, the column prepared with anchored Argonaute:guide molecule complexes. Note that the column can be prepared with Argonaute:guide complexes having different targets. Having passed through the column, the sample is now depleted of the target nucleic acids.

FIG. 3 shows the use of Argonaute:guide molecule complexes as DNA cleaving enzymes and as an in vitro RNA transcript trimming enzyme. (A) A double stranded plasmid is linearized for cloning. The Argonaute:guide complex can be designed to target any sequence, and once bound, will cleave one of the DNA strands. Using two Argonaute:guide complexes, a plasmid can be linearized at a specific nucleotide to give custom “sticky ends” for increased ligation efficiency. (B) Precise excision can be designed by preparing four different Argonaute:guide complexes, giving rise to an excised DNA fragment that has nucleotide-specific cuts and custom “sticky ends” for increased ligation efficiency. Finally, (C), Argonaute:guide complexes can be used to precisely “clean up” in vitro-transcribed RNA by eliminating n+l's from the terminus.

FIG. 4 shows single-molecule analysis of nucleic acid-guided Argonaute proteins. (A) Single-molecule strategy to measure RNA- or DNA-guided Argonaute interaction with RNA or DNA targets. (B) Photobleaching of a target labeled with a single Alexa647 dye is indistinguishable from target cleavage. In contrast, the stepwise photobleaching of a target with 17 Alexa647 dyes is readily distinguished from target. (C) Michaelis-Menten analysis of target cleavage using a standard RNA guide versus a 3′ Alexa555-labeled RNA guide. Values are mean±S.D. (n=3). (D) A trace of an individual molecule of target RNA undergoing RNAi. dark gray: 5′-tethered, 3′ (Alexa647×17)-labeled RNA target, fully complementary to let-7a; light gray: mouse AGO2-RISC programmed with 3′ Alexa555-labeled let-7a RNA guide. Above trace at left, colored bars summarize the species observed. This color code is used throughout the Figures in the rastergrams. (E) Rastergram representation of let-7a-guided AGO2 binding and cleaving a fully complementary RNA target. The rastergram presents 426 individual RNA target molecules, each in a single row.

FIG. 5 shows Argonaute accelerates guide binding to target, compared to nucleic acid alone. (A) Comparison of target binding rates (kon) by 21 nucleotide RNA-guided mouse AGO2- and 16 nucleotide DNA-guided Argonaute:guide molecule complex versus the RNA or DNA guide strands alone. Cumulative binding fraction plots are accompanied by the fluorescence intensity trace for a representative individual molecule. light gray arrowheads: photobleaching of the Alexa555 guide strand; dark gray arrowheads: stepwise photobleaching of a single Alexa647 group; scissors: Argonaute-catalyzed target cleavage; dark gray F: Förster resonance energy transfer from the Alexa555 guide to the Alexa647 target bearing 17 dye moieties. (B) Comparison of kon values for mouse AGO2-let-7a and TtAgo-let-7a. For mouse AGO2 paradigmatic RNA targets, values are mean±S.D. (n=3) and were collected for >1,000 individual molecules. Data for all other pairings were from several hundred individual molecules and error corresponds to the error of fit. All kon values were corrected for the rate of non-specific binding to the slide surface.

FIG. 6 shows mismatches highlight the role of the seed sequence in target binding. Comparison of the kinetic properties of let-7a-guided mouse AGO2-RISC with different let-7a target pairings. Values were derived from data collected from several hundred individual RNA target molecules; error is the error of fit. All kon and koff values were corrected for the rate of non-specific binding to the slide surface. The koff and KD values for nucleic acid in the absence of protein were predicted from the measured kon and the ΔG37° C. calculated by nearest neighbor analysis. koff was not determined for the fully complementary target because it was cleaved under our experimental conditions.

FIG. 7 shows let-7a binds tightly to the seed-matching, 3′ product of target cleavage. (A) A trace of an individual molecule of a 3′-tethered target fully complementary to let-7a. The trace shows that mouse let-7a-RISC bound (dark gray bar), cleaved the target, and then remained bound to the 3′ product (red bar). Finally, RISC departed or the Alexa555 on the guide photobleached. The 3′ cleavage product containing the seed remains on the slide surface, allowing a new molecule of RISC to bind it. (B) Rastergrams comparing 5′-tethered (426 individual molecules) and 3′-tethered (452 individual molecules) RNA targets, fully complementary to let-7a.

FIG. 8 shows AGO2-catalyzed cleavage and product release. (A) Global fit analysis of 5′- and 3′-tethered targets for AGO2 guided by let-7a or miR-21. (B) The detailed kinetic scheme used for global fitting. Rate values are color-coded according to (A). Percentages in parentheses report the proportion of molecules of that product released first.

FIG. 9 shows the plasmids and guides used in the examples to for site-specific cleavage of double-stranded DNA with TtAgo. (A) The 5481 bp plasmid pET GFP LIC cloning vector (u-msfGFP; “plasmid 1”) (B) The 2858 bp plasmid pET empty polycistronic destination vector (2Z′ “plasmid 2”) and (C) the guide molecules A-D are shown hybridizing to their nucleic acid targets.

FIG. 10 shows the results of cleavage reactions containing TtAgo, guided by two pairs of 16 nucleotide single-stranded DNA guides as shown in FIG. 9C, and double-stranded plasmid target DNA. L, 1 kbp double-stranded DNA markers; M1, untreated target plasmid; M2, target plasmid DNA linearized with PvuII. (A) shows cleavage of plasmid 1 in a buffer of 18 mM HEPES-KOH, pH 7.5 at 22° C., 50 mM sodium chloride, 3 mM MnCl₂, 0.01% IGEPAL CA-630 (w/v), 5 mM dithiothreitol, 10% (w/v) glycerol (“buffer 1”) using the guides shown in FIG. 9C. (B) shows cleavage of plasmid 2 in the same buffer used in FIG. 10A and using the same guides described in FIG. 9C. (C) shows cleavage of plasmid 1 in a buffer solution of 18 mM HEPES-KOH, pH 7.5 at 22° C., 75 mM mono-sodium glutamate, 3 mM MnCl₂, 0.01% (w/v) IGEPAL CA-630, 5 mM dithiothreitol, 10% (w/v) glycerol (“buffer 2”) using the guides described in FIG. 9C.

FIG. 11 shows the results of experiments testing guide-length variation for site-specific cleavage of double-stranded DNA with TtAgo. Controls including both target only and target with TtAgo (no guides) and cleavage reactions containing TtAgo, guided by two pair of small, single-stranded DNA ranging in length from 9 nucleotides to 21 nucleotides (nt) and double-stranded plasmid 1 target DNA were incubated for the indicated times (minutes). M, target plasmid DNA linearized with PvuII; SC=supercoiled plasmid; OC=open circle plasmid.

FIG. 12 shows the results of cleaving plasmid 1 with the guides shown in FIG. 9 and TtAgo in different buffers. (A) shows cleavage of plasmid 1 in buffer 1 using 12 nucleotide guides; (B) shows cleavage of plasmid 1 in buffer 2 using 12 nucleotide guides, and (C) shows cleavage of plasmid 1 in a buffer of 10 mM Tris-HCl pH 8, 125 mM NaCl and 0.5 mM MnCl₂ using 21 nucleotide guides.

FIG. 13 shows the results of cleaving plasmid 1 with guide molecules as shown in FIG. 9C except at the indicated positions, counted from the 5′ end of the guide sequence, are identical sequences to target (i.e., mismatches (“MM”). L, 1 kbp double-stranded DNA markers; M, target plasmid 1 DNA linearized with PvuII.

FIG. 14 shows a schematic for using Argonaute:guide molecule complex for nucleic acid cloning wherein the removed segments have identical cleavage sites.

FIG. 15 shows a schematic for using Argonaute:guide molecule complex for nucleic acid cloning wherein the removed segments have distinct cleavage sites and are cloned using bridge oligonucleotides.

FIG. 16 shows the results of using Argonaute:guide molecule complexes as a probe in fixed cells. FIG. 14.1 shows TtAgo in complex with the telomeric DNA guide staining in U2OS cells. FIG. 14.2 shows telomeric DNA guide alone staining in U2OS cells. FIG. 14.3 shows TtAgo in complex with the telomeric DNA guide staining in RPE-1 cells. FIG. 14.4 shows telomeric DNA guide alone staining in RPE-1 cells. FIG. 14.5 shows TtAgo in complex with the random DNA staining in U2OS cells. FIG. 14.6 shows random DNA guide alone staining in U2OS cells. FIG. 14.7 shows TtAgo in complex with the random DNA guide staining in RPE-1 cells. FIG. 14.8 shows random DNA guide alone staining in RPE-1 cells.

### DETAILED DESCRIPTION

Provided herein are methods and compositions using Argonaute:guide molecule complexes as highly specific probes, as a means to capture specific DNA or RNA molecules (e.g., to purify a specific DNA or RNA molecule), and as highly specific, “custom-designed” nucleic acid enzymes. The inventors have discovered optimal conditions for specific binding and cleavage using Argonaute:guide molecule complexes, including unexpected findings regarding the useful and optimal length of guide molecules.

Argonaute:guide molecule complexes have a number of advantageous features. For example, when a single stranded guide nucleic acid molecule is combined with an Argonaute polypeptide, the complex stably and specifically binds its target. Because the guide molecule can be designed to bind any sequence, the disclosed methods and compositions can be used to bind any sequence, whether its origin is prokaryotic, eukaryotic, or viral. Since the Argonaute:guide molecule complexes have nucleic acid cleavage activity and they can be designed to bind any sequence, Argonaute:guide molecule complexes can be used in methods of cleaving nucleic acids at almost any given sequence; unlike currently available endonucleases, which are both DNA double stranded-dependent and sequence-dependent.

The guide molecule can be divided into two domains, a recruiting (or seed) domain (nucleotide positions g1-g8, with nucleotides g2-g8 seeming to be responsible for recruiting activity), and a stabilization domain. The recruiting domain helps the Argonaute:guide molecule complexes to identify the RNA or DNA target sequence and speed up the process of binding to the target RNA or DNA; the stabilization domain appears to provide further complementarity to the target RNA or DNA to stabilize binding and to allow for temperature-dependent cleavage. Prokaryotic guide molecules are about 16 nucleotides long in vivo and eukaryotic guide molecules are about 21 nucleotides long in vivo. In contrast, the inventors have found that guide molecules as small as 12 nucleotides permit function, and in some cases, are preferable to the longer guide molecules found in vivo in the disclosed methods.

Using Argonaute from Thermus thermophilus (TtA), the inventors also observed that binding occurred 10-300 times faster when the guide molecule was complexed with TtA than alone, and that the binding was extremely stable, having a dissociation constant (KD) of <1 nM for perfect pairing to single stranded RNA (ssRNA) or single-stranded DNA (ssDNA).

In contrast to restriction enzymes, TtAgo has been shown to use 21 nucleotide single-stranded DNA guides that direct the Argonaute protein to nick double-stranded DNA on one strand (Swarts et al., 2014. DNA-guided DNA interference by a prokaryotic Argonaute. Nature, 507, 258-261), although in vivo, TtAgo is thought to use 16 nucleotide single-stranded DNA guides. DNA cleavage is mediated by Mg²⁺ or Mn²⁺ and generates fragments with 3′ hydroxy and 5′ monophosphate ends (Wang et al., 2009. Nucleation, propagation and cleavage of target RNAs in Ago silencing complexes. Nature 461, 754-761), the termini required for enzymatic ligation of DNA. The inventors have surprisingly found that in contrast to the guide molecules found in vivo, shorter guide molecules (e.g., 12-15 nucleotides) give optimal cleavage results in vitro in appropriate buffers without generating side-products.

The inventors also discovered that Argonaute:guide molecule complexes comprising a detectable moiety (such as a fluorescent dye) at the 3′ end of the guide molecule bind to target nucleic acid sequences at physiologic temperatures and can be used in imaging, diagnostic and preparative applications.

### DEFINITIONS

An “affinity tag” refers to either a peptide affinity tag or a nucleic acid affinity tag. Affinity tag generally refers to a protein or nucleic acid sequence that can be bound to a molecule (e.g., bound by a small molecule, protein, covalent bond). An affinity tag can be a non-native sequence. A peptide affinity tag can comprise a peptide. A peptide affinity tag can be one that is able to be part of a split system (e.g., two inactive peptide fragments can combine together in trans to form an active affinity tag). A plurality of affinity tags can be fused to a native protein or nucleotide sequence. A nucleic acid affinity tag can comprise a nucleic acid, such as a sequence that can selectively bind to a known nucleic acid sequence (e.g. through hybridization). A nucleic acid affinity tag can be a sequence that can selectively bind to a protein. An affinity tag can be introduced using methods of in vitro or in vivo transcription. An affinity tag can be fused to a nucleotide sequence. Nucleic acid affinity tags can include, for example, a chemical tag, an RNA-binding protein binding sequence, a DNA-binding protein binding sequence, a sequence hybridizable to an affinity-tagged polynucleotide, a synthetic RNA aptamer, a synthetic DNA aptamer, or an aptazyme. Examples of chemical nucleic acid affinity tags include nucleotriphosphates containing biotin (which binding partner is avidin or streptavidin), fluorescent dyes, and digoxegenin. Examples of protein-binding nucleic acid affinity tags include restriction endonuclease binding sequences, transcription factor binding sequences, zinc finger binding sequences, TALEN binding sequences, or any sequence recognized by a DNA binding protein. Examples of protein-binding nucleic acid affinity tags include the MS2 binding sequence, the U1A binding sequence, stem-loop binding protein sequences, the boxB sequence, the eIF4A sequence, or any sequence recognized by an RNA binding protein. Examples of nucleic acid affinity-tagged oligonucleotides include biotinylated oligonucleotides, 2,4-dinitrophenyl oligonucleotides, fluorescein oligonucleotides, and primary amine-conjugated oligonucleotides

“Argonaute” generally refers to a polypeptide with at least about 5%, 10%, 20%, 30%, 40%, 50%, 60%, 70%, 80%, 90%, 95%, or 100% sequence identity to a wild type Argonaute polypeptide (e.g., Argonaute from Thermus thermophilus, SEQ ID NO:1, Table 1). Argonaute can be an Aquifex aeolicus, a Microsystis aeruginosa, a Clostridium bartlettii, an Exiguobacterium, an Anoxybacillus flavithermus, a Halogeometricum borinquense, a Halorubrum lacusprofundi, an Aromatoleum aromaticum, a Thermus thermophilus, a Synechococcus, a Synechococcus elongatus, or a Thermosynechococcus elogatus Argonaute. Argonaute can be mammalian Argonaute, such as mouse AGO2. Argonaute can refer to the wild-type or a modified form of the Argonaute protein that can comprise an amino acid change such as a deletion, insertion, substitution, variant, mutation, fusion, chimera, or any combination thereof.

TABLE 1
Argonaute polypeptide sequence from T.
thermophilus (SEQ ID NO: 1)

```
Met Asn His Leu Gly Lys Thr Glu Val Phe Leu Asn
1 5 10
Arg Phe Ala Leu Arg Pro Leu Asn Pro Glu Glu Leu
                15 20
Arg Pro Trp Arg Leu Glu Val Val Leu Asp Pro Pro
25 30 35
Pro Gly Arg Glu Glu Val Tyr Pro Leu Leu Ala Gln
                        40 45
Val Ala Arg Arg Ala Gly Gly Val Thr Val Arg Met
        50 55 60
Gly Asp Gly Leu Ala Ser Trp Ser Pro Pro Glu Val
                                65 70
Leu Val Leu Glu Gly Thr Leu Ala Arg Met Gly Gln
                75 80
Thr Tyr Ala Tyr Arg Leu Tyr Pro Lys Gly Arg Arg
85 90 95
Pro Leu Asp Pro Lys Asp Pro Gly Glu Arg Ser Val
                        100 105
Leu Ser Ala Leu Ala Arg Arg Leu Leu Gln Glu Arg
        110 115 120
Leu Arg Arg Leu Glu Gly Val Trp Val Glu Gly Leu
                                125 130
Ala Val Tyr Arg Arg Glu His Ala Arg Gly Pro Gly
                135 140
Trp Arg Val Leu Gly Gly Ala Val Leu Asp Leu Trp
145 150 155
Val Ser Asp Ser Gly Ala Phe Leu Leu Glu Val Asp
                        160 165
Pro Ala Tyr Arg Ile Leu Cys Glu Met Ser Leu Glu
        170 175 180
Ala Trp Leu Ala Gln Gly His Pro Leu Pro Lys Arg
                                185 190
Val Arg Asn Ala Tyr Asp Arg Arg Thr Trp Glu Leu
                195 200
Leu Arg Leu Gly Glu Glu Asp Pro Lys Glu Leu Pro
205 210 215
Leu Pro Gly Gly Leu Ser Leu Leu Asp Tyr His Ala
                        220 225
Ser Lys Gly Arg Leu Gln Gly Arg Glu Gly Gly Arg
        230 235 240
Val Ala Trp Val Ala Asp Pro Lys Asp Pro Arg Lys
                                245 250
Pro Ile Pro His Leu Thr Gly Leu Leu Val Pro Val
                255 260
Leu Thr Leu Glu Asp Leu His Glu Glu Glu Gly Ser
265 270 275
Leu Ala Leu Ser Leu Pro Trp Glu Glu Arg Arg Arg
                        280 285
Arg Thr Arg Glu Ile Ala Ser Trp Ile Gly Arg Arg
        290 295 300
Leu Gly Leu Gly Thr Pro Glu Ala Val Arg Ala Gln
                                305 310
Ala Tyr Arg Leu Ser Ile Pro Lys Leu Met Gly Arg
                315 320
Arg Ala Val Ser Lys Pro Ala Asp Ala Leu Arg Val
325 330 335
Gly Phe Tyr Arg Ala Gln Glu Thr Ala Leu Ala Leu
                        340 345
Leu Arg Leu Asp Gly Ala Gln Gly Trp Pro Glu Phe
        350 355 360
Leu Arg Arg Ala Leu Leu Arg Ala Phe Gly Ala Ser
                                365 370
Gly Ala Ser Leu Arg Leu His Thr Leu His Ala His
                375 380
Pro Ser Gln Gly Leu Ala Phe Arg Glu Ala Leu Arg
385 390 395
Lys Ala Lys Glu Glu Gly Val Gln Ala Val Leu Val
                        400 405
Leu Thr Pro Pro Met Ala Trp Glu Asp Arg Asn Arg
        410 415 420
Leu Lys Ala Leu Leu Leu Arg Glu Gly Leu Pro Ser
                                425 430
Gln Ile Leu Asn Val Pro Leu Arg Glu Glu Glu Arg
                435 440
His Arg Trp Glu Asn Ala Leu Leu Gly Leu Leu Ala
445 450 455
Lys Ala Gly Leu Gln Val Val Ala Leu Ser Gly Ala
                        460 465
Tyr Pro Ala Glu Leu Ala Val Gly Phe Asp Ala Gly
        470 475 480
Gly Arg Glu Ser Phe Arg Phe Gly Gly Ala Ala Cys
                                485 490
Ala Val Gly Gly Asp Gly Gly His Leu Leu Trp Thr
                495 500
Leu Pro Glu Ala Gln Ala Gly Glu Arg Ile Pro Gln
505 510 515
Glu Val Val Trp Asp Leu Leu Glu Glu Thr Leu Trp
                        520 525
Ala Phe Arg Arg Lys Ala Gly Arg Leu Pro Ser Arg
        530 535 540
Val Leu Leu Leu Arg Asp Gly Arg Val Pro Gln Asp
                                545 550
Glu Phe Ala Leu Ala Leu Glu Ala Leu Ala Arg Glu
                555 560
Gly Ile Ala Tyr Asp Leu Val Ser Val Arg Lys Ser
565 570 575
Gly Gly Gly Arg Val Tyr Pro Val Gln Gly Arg Leu
                        580 585
Ala Asp Gly Leu Tyr Val Pro Leu Glu Asp Lys Thr
        590 595 600
Phe Leu Leu Leu Thr Val His Arg Asp Phe Arg Gly
                                605 610
Thr Pro Arg Pro Leu Lys Leu Val His Glu Ala Gly
                615 620
Asp Thr Pro Leu Glu Ala Leu Ala His Gln Ile Phe
625 630 635
His Leu Thr Arg Leu Tyr Pro Ala Ser Gly Phe Ala
                        640 645
Phe Pro Arg Leu Pro Ala Pro Leu His Leu Ala Asp
        650 655 660
Arg Leu Val Lys Glu Val Gly Arg Leu Gly Ile Arg
                                665 670
His Leu Lys Glu Val Asp Arg Glu Lys Leu Phe Phe
                675 680
Val
685
```

An Argonaute can refer to any modified (e.g., shortened, mutated, lengthened) polypeptide sequence or homologue of the Argonaute. An Argonaute polynucleotide can be codon optimized for a particular organism. An Argonaute can be enzymatically inactive, partially active, constitutively active, fully active, inducibly active and/or more active, (e.g. more than the wild type homologue of the protein or polypeptide).

“Cell” includes prokaryotic cells and eukaryotic cells.

“Complementarity” refers to the ability of nucleotides, or analogues thereof, to form Watson-Crick base pairs. Complementary nucleotide sequences will form Watson-Crick base pairs and non-complementary nucleotide sequences will not.

“Guide molecule,” and “guide strand” refer to a single-stranded oligonucleotide that comprises at least 12 nucleotides and is capable of directing an Argonaute polypeptide:guide molecule complex to a target polynucleotide. The guide molecule can be a DNA or an RNA molecule. The guide molecule binds an Argonaute protein of the disclosure and hybridizes to a target nucleic acid. Guide molecule nucleotides can be numbered from 5′ to 3′, with the initial nucleotide being indicated as g1, and subsequent nucleotides being indicated, proceeding 5′ to 3′, as g2, g3, g4, etc. Guide molecules are discussed further below.

“Oligonucleotide” refers to a polymer of nucleotides comprising naturally occurring nucleotides, non-naturally occurring nucleotides, derivatized nucleotides, or a combination thereof.

“RNA,” “RNA molecule,” or “ribonucleic acid molecule” refers to a polymer of ribonucleotides. “DNA,” “DNA molecule,” or “deoxyribonucleic acid molecule: refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains.

“Sample” generally refers to a sample from a biological entity. A sample can comprise nucleic acid. The nucleic acid can be purified and/or enriched. Other components may also be purified or enriched in a sample, such as specific or a class of molecules, such as proteins, mRNA molecules, DNA molecules, etc. Samples can come from various sources. Examples of samples include blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, check swab, breath, hair, finger nails, skin, biopsy, placental fluid, amniotic fluid, cord blood, lymphatic fluids, cavity fluids, sputum, pus, microbiota, meconium, breast milk, buccal samples, nasopharyngeal wash, other excretions or bodily fluids, or any combination thereof. Samples can originate from tissues. Examples of tissue samples include connective tissue, muscle tissue, nervous tissue, epithelial tissue, cartilage, cancerous or tumor sample, bone marrow, or bone. The sample can be provided from a human or animal. The sample may be provided from a mammal, vertebrate, such as murines, simians, humans, farm animals, sport animals, or pets. Samples can also originate from cell lysates. Samples can include cultured cells (prokaryotic and eukaryotic). Samples can include viruses. In some cases, a sample is assembled from partially-purified or purified components.

“Specific” refers to an interaction of two molecules where one of the molecules through, for example chemical or physical means, specifically binds to the second molecule. Exemplary specific binding interactions can refer to antigen-antibody binding, avidin-biotin binding, carbohydrates and lectins, complementary nucleic acid sequences (e.g., hybridizing), complementary peptide sequences including those formed by recombinant methods, effector and receptor molecules, enzyme cofactors and enzymes, enzyme inhibitors and enzymes, and the like. “Non-specific” can refer to an interaction between two molecules that is not specific.

“Target nucleic acid,” “target polynucleotide,” and “target nucleotide sequence” generally refer to a target nucleic acid to be targeted in the methods of the invention. A target nucleic acid can refer to a chromosomal sequence or an extrachromosomal sequence, (e.g. an episomal sequence, a minicircle sequence, a plasmid, a mitochondrial sequence, a chloroplast sequence, etc.). A target nucleic acid can be a double-stranded or single-stranded DNA; a target nucleic acid may also be an RNA.

The instant disclosure provides methods and compositions using Argonaute:guide molecule complexes as probes, purification aids, and DNA and RNA cutting enzymes.

### Argonaute:Guide Molecule Complexes as Probes

One embodiment comprises a method of recruiting an Argonaute polypeptide to a heterologous target RNA or DNA sequence comprising combining the Argonaute polypeptide with a heterologous, single-stranded oligonucleotide guide molecule that comprises a recruiting domain of at least 8 nucleotides at its 5′ end of the guide molecule (g1-g8) and a stabilization domain adjacent and 3′ to the recruiting domain and comprising at least 4 nucleotides (g9-g12), wherein the stabilization domain of the guide molecule has sufficient complementarity to the target RNA or DNA sequence such that the Argonaute polypeptide:guide molecule complex stably binds to the target RNA or DNA sequence. Once recruited, the Argonaute:guide molecule complex can be detected; thus methods of detecting Argonaute:guide molecule complexes are included; such methods can be used to detect and quantify, for example, RNA in a sample.

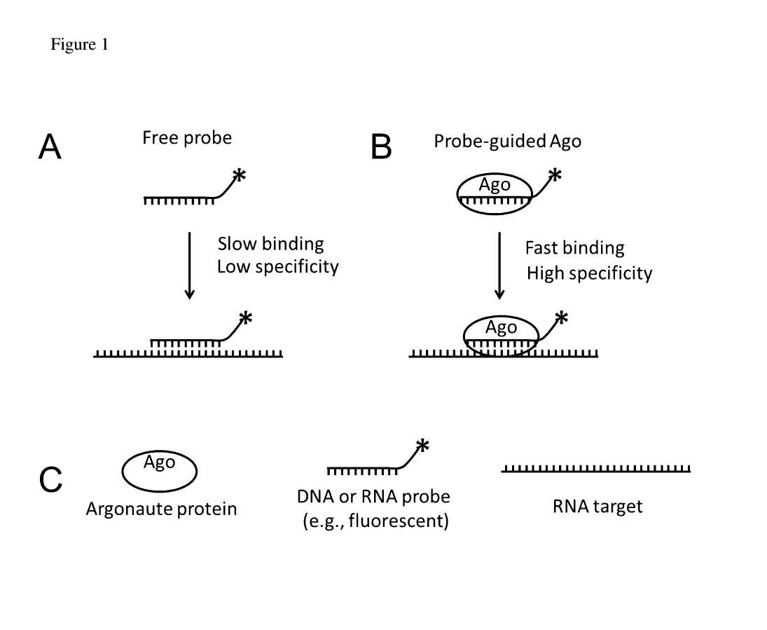

In some embodiments, the Argonaute:guide molecule complexes of the invention are able to bind their target polynucleotides 10 to 300 times faster than the guide molecule binding the target polynucleotide alone. The binding of a guide molecule by itself is illustrated in FIG. 1A; the binding of an Argonaute:guide molecule complex is shown in FIG. 1B. Once bound, the Argonaute:guide molecule complexes may have dissociation constants of less than 1 nM.

### Guide Molecules

Any RNA or DNA sequence can be bound by Argonaute:guide molecule complexes provided that a suitable guide molecule can be designed. As noted above, guide molecules are at least about 12 nucleotides long and can be RNA or DNA molecules. Guide molecules have two functional domains. A first domain, 5′ of the molecule, can be thought of as a recruiting domain, with positions g2-g8 being responsible for this activity. This domain is used to target a sequence on an RNA or DNA molecule. The second domain, a stabilization domain, is at the 3′ end and is at least 4 nucleotides long, and has a role in stabilizing the interaction between the guide strand and its complementary target when complexed with an Argonaute polypeptide. At least positions g9-g12 are responsible for this activity, although some engineered guide strands will have less than 100% complementarity to its target sequence, such as about 38%-100% complementarity, including about 38%, 50%, 63%, 75%, 88%, and 100% complementarity. A stretch of nucleotides of the guide molecule can be complementary to the target nucleic acid (e.g., hybridizable). A stretch of at least 8, 9, 10, 11, 12, 13, 14, 15, 16, 17, 18, 19, 20, 21, 22, 23, 24, 25, 25, 27, 28, 29, or 30 contiguous nucleotides can be complementary to target nucleic acid. Preferably, the guide molecule is phosphorylated at its 5′ end. A guide molecule may have additional sequences appended to its 3′ end. A guide molecule may be from 12 to about 100 nucleotides long or more (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more). These additional sequences can be exploited as a means to detect the guide molecule, such as through hybridization or multiplexing assays. However, in other applications, such as using Argonaute:guide molecule complexes to cleave nucleic acids, shorter length guide molecules are preferable.

The guide molecule can hybridize to a target nucleic acid. The guide molecule can hybridize with a mismatch between the guide molecule and the target nucleic acid. A guide molecule can comprise at least 1, 2, 3, 4, 5, 6, 7, or 8 or more mismatches when hybridized to a target nucleic acid. Guide molecules can tolerate few mismatches in the recruiting domain, although some tolerance is possible at g6, g7, and g8. In the stabilization domain, there can be about 1, 2, 3, 4, or 5 mismatches (depending on the length of the guide molecule; shorter guide molecules with only 4 nucleotides in the stabilization domain may tolerate 3 or fewer mismatches). Such mismatches can be anywhere in the stabilization domain, but preferably at the 3′ end of the molecule. For example, positions g6-g16, such as g6, g7, g8, g9, g10, g11, g12, g13, g14, g15, and g16 or any combination thereof, can be mismatched in 16 nucleotide long guide molecules. Mismatches in the recruiting domain can have mismatches preferably in positions g6, g7, and/or g8 (See Examples).

A guide molecule can comprise one or more modifications (e.g., a base modification, a backbone modification), to provide the nucleic acid with a new or enhanced feature (e.g., improved stability). A guide molecule can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming guide molecules, the phosphate groups can covalently link adjacent nucleosides to one another to form a linear polymeric compound. In addition, linear compounds may have internal nucleotide base complementarity and may therefore fold in a manner as to produce a fully or partially double-stranded compound. Within guide molecules, the phosphate groups can commonly be referred to as forming the internucleoside backbone of the guide molecule. The linkage or backbone of the guide molecule can be a 3′ to 5′ phosphodiester linkage.

A guide molecule can comprise nucleoside analogs, which are oxy- or deoxy-analogues of the naturally-occurring DNA and RNA nucleosides deoxycytidine, deoxyuridine, deoxyadenosine, deoxyguanosine and thymidine. A guide molecule can also include a universal base, such as deoxyinosine, or 5-nitroindole.

A guide molecule can comprise a modified backbone and/or modified internucleoside linkages. Modified backbones can include those that retain a phosphorus atom in the backbone and those that do not have a phosphorus atom in the backbone.

Suitable modified guide molecule backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable guide molecules having inverted polarity can comprise a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

A guide molecule can comprise one or more phosphorothioate and/or heteroatom internucleoside linkages, in particular —CH₂—NH—O—CH₂—, —CH₂—N(CH₃)—O—CH₂— (i.e. a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—).

A guide molecule can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A guide molecule can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A guide molecule can comprise a nucleic acid mimetic. The term “mimetic” includes polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A guide molecule can comprise linked morpholino units (i.e. morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of guide molecules. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA 4,4′-dimethoxytrityl (DMT) protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bridged bicyclic sugar moiety. The linkage can be a methylene (—CH₂—), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties. Another useful modification includes unlocked nucleic acid (UNA) monomers, which are acyclic derivatives of RNA lacking the C2′-C3′-bond of the ribose ring of RNA. The missing bond increases the flexibility of the molecule, decreasing duplex thermo stability.

A guide molecule can comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)—ONH₂, and O(CH₂)ₙON((CH₂)ₙCH₃)₂, where n and m are from 1 to about 10. A sugar substituent group can be selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an guide molecule, or a group for improving the pharmacodynamic properties of a guide molecule, and other substituents having similar properties. A suitable modification can include 2′-methoxyethoxy (2′-O—CH₂CH₂OCH₃, also known as 2′-O-(2-methoxyethyl) or 2′-MOE i.e., an alkoxyalkoxy group). A further suitable modification can include 2′-dimethylaminooxyethoxy, (i.e., a O(CH₂)₂O N(CH₃)₂ group, also known as 2′-DMAOE), and 2′-dimethylaminoethoxyethoxy (also known as 2′-O-dimethyl-amino-ethoxy-ethyl or 2′-DMAEOE), i.e., 2′-O—(CH₂)₂—O—(CH₂)₂—N(CH₃)₂.

Other suitable sugar substituent groups can include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 3′-arabino modification is 2′-F. Similar modifications may also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds may also have sugar mimetics such as cyclobutyl moieties in place of the pentofuranosyl sugar.

A guide molecule may also include nucleobase (often referred to simply as “base”) modifications or substitutions. “Unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminopurine, 2,6-diaminopurine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil found in pseudouridine), 5-hydroxybutynl-2′-deoxyuridine, 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,2′:4,5)pyrrolo(2,3-d)pyrimidin-2-one).

Heterocyclic base moieties can include those in which the purine or pyrimidine base is replaced with other heterocycles, for example 7-deaza-adenine, 7-deazaguanosine, 8-aza-7-deazaguanosine, 2-aminopyridine, and 2-pyridone; isoG and isoC and hydrophobic non-natural bases such thioisoquinolines/isocarbostyrils (SICS) (Seo et al. Journal of American Chemical Society 2009 p 3246-52). Nucleobases can be useful for increasing the binding affinity of a polynucleotide compound. These can include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

In some embodiments, the guide molecules comprise one or more sugar modifications (2′), such as a 2′-O—CH₃, a 2′-F, a 2′-MOE modification. In some embodiments, guide molecules can comprise one or more modified bases, such as a LNA, a UNA, deoxyuridine, pseudouridine, 5-methylcytosine, 2-aminopurine, 2,6-diaminopurine, deoxyinosine, 5-hydroxybutynl-2′-deoxyuridine, 8-aza-7-deazaguanosine, or 5-nitroindole. In some embodiments, guide molecules comprise one or more sugar modifications and one or more modified bases.

A modification of a guide molecule can comprise chemically linking to the guide molecule one or more moieties or conjugates that can enhance the activity, cellular distribution or cellular uptake of the guide molecule. These moieties or conjugates can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties include lipid moieties such as a cholesterol moiety, cholic acid, a thioether (e.g., hexyl-S-tritylthiol), a thiocholesterol, an aliphatic chain (e.g., dodecanediol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety.

A modification may include a “protein transduction domain” or PTD. The PTD can refer to a polypeptide, polynucleotide, carbohydrate, or organic or inorganic compound that facilitates traversing a lipid bilayer, micelle, cell membrane, organelle membrane, or vesicle membrane. A PTD can be attached to another molecule, which can range from a small polar molecule to a large macromolecule and/or a nanoparticle, and can facilitate the molecule traversing a membrane, for example going from extracellular space to intracellular space, or cytosol to within an organelle. A PTD can be covalently linked to the amino terminus or carboxy terminus of a polypeptide. A PTD can be covalently linked to a nucleic acid. Exemplary PTDs include a minimal peptide protein transduction domain; a polyarginine sequence comprising a number of arginines sufficient to direct entry into a cell (e.g., 3, 4, 5, 6, 7, 8, 9, 10, or 10-50 arginines), a VP22 domain, a Drosophila Antennapedia protein transduction domain, a truncated human calcitonin peptide, polylysine, and transportan, arginine homopolymer of from 3 arginine residues to 50 arginine residues. The PTD can be an activatable cell penetrating peptide (ACPP). ACPPs can comprise a polycationic cell penetrating peptide (e.g., Arg9 or “R9”) connected via a cleavable linker to a matching polyanion (e.g., Glu9 or “E9”), which can reduce the net charge to nearly zero and thereby inhibit adhesion and uptake into cells. Upon cleavage of the linker, the polyanion can be released, locally unmasking the polyarginine and its inherent adhesiveness, thus “activating” the ACPP to traverse the membrane.

It is not necessary for all positions in a given oligonucleotide to be uniformly modified, and in fact more than one of the aforementioned modifications may be incorporated in a single oligonucleotide or even at within a single nucleoside within an oligonucleotide.

### Argonaute Proteins

The Argonaute polypeptide can be from a prokaryote or a eukaryote. A eukaryotic Argonaute can include mouse Argonautes, such as AGO2. The Argonaute may be derived from an archaea or a bacterium. The bacterium may be selected from a thermophilic bacterium and a mesophilic bacterium. The bacteria or archaea may be selected from Aquifex aeolicus, Pyrococcus furiosus, Microsystis aeruginosa, Clostridium bartlettii, Exiguobacterium, Anoxybacillus flavithermus, Halogeometricum borinquense, Halorubrum lacusprofundi, Aromatoleum aromaticum, Thermus thermophilus, Synechococcus, Synechococcus elongatus, and Thermosynechococcus elogatus, or any combination thereof. In some embodiments, Argonaute polypeptide comprises at least 20% amino acid sequence identity to Argonaute from T. thermophilus. In some embodiments, the Argonaute polypeptide comprises at least 60%, 70%, 80%, 90%, 95%, and 100% amino acid sequence identity to Argonaute from T. thermophilus.

In some embodiments, the Argonaute polypeptide further comprises an affinity tag or other additional polypeptide sequence, such as a nuclear localization sequence (reviewed in Marfori et al., Biochimica et Biophsyica Acta 1813:1562-1577, 2011 and Lange et al., J. Biol. Chem. 282:5101-5105), a mitochondrial localization sequence, and a chloroplast localization sequence.

In some embodiments, the Argonaute is a type I prokaryotic Argonaute. In some embodiments, the type I prokaryotic Argonaute carries a DNA nucleic acid-targeting nucleic acid. In some embodiments, the DNA nucleic acid-targeting nucleic acid targets one strand of a double stranded DNA (dsDNA) to produce a nick or a break of the dsDNA. The nick or break can trigger host DNA repair. In some embodiments, the host DNA repair is non-homologous end joining (NHEJ) or homologous directed recombination (HDR). In some embodiments, the dsDNA is selected from a genome, a chromosome, and a plasmid. The type I prokaryotic Argonaute can be a long type I prokaryotic Argonaute, which may possess an N-PAZ-MID-PIWI domain architecture. In some embodiments the long type I prokaryotic Argonaute possesses a catalytically active PIWI domain. The long type I prokaryotic Argonaute can possess a catalytic tetrad encoded by aspartate-glutamate-aspartate-aspartate/histidine (DEDX). The catalytic tetrad can bind one or more magnesium ions or manganese ions. In some embodiments, the type I prokaryotic Argonaute anchors the 5′ phosphate end of a DNA guide. In some embodiments, the DNA guide has a deoxy-cytosine at its 5′ end. In some embodiments, the type I prokaryotic Argonaute is a Thermus thermophilus Ago (TtAgo).

Yet in other embodiments, the prokaryotic Argonaute is a type II Ago. The type II prokaryotic Argonaute can carry an RNA nucleic acid-targeting nucleic acid. The RNA nucleic acid-targeting nucleic acid can target one strand of a double stranded DNA (dsDNA) to produce a nick or a break of the dsDNA which may trigger host DNA repair; the host DNA repair can be non-homologous end joining (NHEJ) or homologous directed recombination (HDR). In some embodiments, the dsDNA is selected from a genome, a chromosome and a plasmid. The type II prokaryotic Argonaute may be a long type II prokaryotic Argonaute and a short type II prokaryotic Argonaute. A long type II prokaryotic Argonaute may have an N-PAZ-MID-PIWI domain architecture. A short type II prokaryotic Argonaute may have a MID and PIWI domain, but not a PAZ domain. In some embodiments, the short type II Ago has an analog of a PAZ domain. In some embodiments the type II Ago does not have a catalytically active PIWI domain. The type II Ago may lack a catalytic tetrad encoded by aspartate-glutamate-aspartate-aspartate/histidine (DEDX). In some embodiments, a gene encoding the type II prokaryotic Argonaute clusters with one or more genes encoding a nuclease, a helicase or a combination thereof. The nuclease or helicase may be natural, designed or a domain thereof. In some embodiments, the nuclease is selected from a Sir2, RE1 and TIR. The type II Ago may anchor the 5′ phosphate end of an RNA guide. In some embodiments, the RNA guide has a uracil at its 5′ end. In some embodiments, the type II prokaryotic Argonaute is a Rhodobacter sphaeroides Argonaute.

In some embodiments, it may be desirable to use an Argonaute polypeptide that has lost its ability to cleave a nucleic acid, such as in applications where the Argonaute:guide molecule complex is used as a probe. In such embodiments, one or more of the amino acid residues in the catalytic domain is substituted or deleted, such that catalytic activity is abolished. In other embodiments, using a cleavage temperature-inducible Argonaute may be desired to control the timing of cleavage, or if cleavage should be inhibited at non-inducible temperatures. An example of a “temperature inducible” Argonaute polypeptide is that from T. thermophilus, which, when complexed with a suitable guide strand, will cleave RNA only at temperatures of 55° C. or higher (up to at least 75° C.), or in the case of cleaving DNA, only at temperatures of 65° C. or higher (up to at least 75° C.).

### Target Polynucleotides

A target nucleic acid may comprise one or more sequences that are at least partially complementary to one or more guide molecules. The target nucleic acid can be part or all of a gene, a 5′ end of a gene, a 3′ end of a gene, a regulatory element (e.g. promoter, enhancer), a pseudogene, non-coding DNA, a microsatellite, an intron, an exon, and chromosomal DNA. Furthermore, the target nucleic acid may comprise DNA or RNA of prokaryotes, eukaryotes, and viruses, and includes DNA and RNA from mitochondria, nuclei, plastids (such as chloroplasts). The target nucleic acid can be part or all of a plasmid DNA. The target nucleic acid can be in vitro or in vivo.

Vectors In certain embodiments, Argonaute polypeptides or guide polynucleotides are expressed from a recombinant vector. Suitable recombinant vectors include DNA plasmids, viral vectors or DNA minicircles. Generation of the vector construct can be accomplished using any suitable genetic engineering techniques well known in the art, including PCR, oligonucleotide synthesis, restriction endonuclease digestion, ligation, transformation, plasmid purification, and DNA sequencing (Sambrook et al. Molecular Cloning: A Laboratory Manual. (1989), Coffin et al. Retroviruses. Plainview, N.Y.: Cold Spring Harbor Laboratory Press (1997) and RNA Viruses: A Practical Approach (Alan J. Cann, Ed., Oxford University Press, (2000)). As will be apparent to one of ordinary skill in the art, a variety of suitable vectors are available for transferring nucleic acids into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including a retrovirus, lentivirus, adenovirus, adeno-associated virus, pox virus or alphavirus. The recombinant vectors can be delivered as described herein, and persist in target cells (e.g., stable transformants).

In certain embodiments, guide molecules used to practice the invention are synthesized in vitro using chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68: 109; Beaucage (1981) Tetra. Lett. 22: 1859; U.S. Pat. No. 4,458,066.

### Argonaute:Guide Molecule Complexes and Solid Supports

In some embodiments, Argonaute:guide molecule complexes are attached to a substrate, or solid support. The configuration of a substrate can be in the form of beads, spheres, particles, granules, a gel, a membrane, or a surface. Surfaces can be planar, substantially planar, or non-planar. Solid supports can be porous or non-porous.

A support or matrix is any material to which a binding molecule is covalently attached. Many substances have been described and utilized as matrices, including agarose (such as cross-linked agarose), cellulose, dextran, polyacrylamide, latex, polystyrene, polyethylene, polypropylene, polyfluoroethylene, and polyethyleneoxy, as well as co-polymers and grafts thereof. Solid supports can also comprise inorganic materials, such as glass, silica, controlled pore glass (CPG), reverse phase silica; or metal, such as gold, iron (such as iron oxide), or platinum. Especially useful supports are those with a high surface area to volume ratio, chemical groups that are easily modified for covalent attachment of binding molecules, minimal nonspecific binding properties, good flow characteristics, and mechanical and chemical stability.

Methods for immobilizing polypeptides on substrates are well-known in the art, and polypeptides can be attached covalently or non-covalently. In most embodiments, the polypeptide is covalently attached to the support. The types of functionalities generally used for attachment include easily reactive components, such as primary amines, sulfhydryls, aldehydes, carboxylic acids, hydroxyls, phenolic groups, and histidinyl residues. Most often the solid support is first activated with a compound that is reactive to one of these functionalities. The activated complex can then form a covalent linkage between the polypeptide and the support, immobilizing the polypeptide on the solid support.

Coupling polypeptides through their amine groups is possible because of the abundance of lysine side chain ε-amines and N-terminal α-amines. Solid supports are prepared to have free aldehyde groups, which can be used to immobilize amine-containing polypeptides by reductive amination. For example, cyanoborohydride or other appropriate mild reducing agent can be used to couple the polypeptide to an aldehyde-prepared support.

In other amine-reactive methods, solid supports are derivatized with an azlactone ring, such as is available from Life Technologies (Grand Island, N.Y.). Another approach is to prepare supports (such as agarose supports) with reactive imidazole carbamates. This method is also appropriate for immobilizing small organic molecules. Other amine-reactive methods include the use of N-Hydroxysuccinimide (NHS)-ester-, periodate and cyanoborohydride-, and cyanogen bromide-activated supports.

Coupling through sulfhydryl groups can have the advantage that coupling can occur at distinct (thiol group) sites on the coupled protein instead of the more ubiquitous amine groups. Such coupling may be advantageous to avoid coupling at binding sites in the polypeptides. Polypeptides, especially polypeptides, can be engineered to include a terminal sulfhydryl group to promote coupling. Supports that have been derivatized with iodo-acetyl groups, preferably at the end of a spacer arm are useful for sulfhydryl-mediated coupling.

As with sulfhydryl group coupling, coupling through carbonyl groups can also have the advantage of localized coupling. Although biological molecules do not usually contain carbonyl ketones or aldehydes, such groups can be created. Glycomolecules (e.g., glycoproteins and glycolipids) often have sugar residues that are adjacent to carbon molecules having hydroxyl groups; these can be periodate-oxidized to create aldehydes. These aldehydes can be linked to supports through immobilized hydrazide, hydrazine, or amine group (by Schiff base formation or reductive animation).

Coupling through carboxyl groups is also useful. Supports containing amines or hydrazides can be used to form amide bonds with carboxylates through carbodiimide-mediated reactions, such as those using the carbodiimide, 1-ethyl-3-(3-dimethylaminopropyl) carbodiimide (EDC).

In some embodiments, the polypeptide can be bound to another molecule that is directly linked to the support. For example, protein A or protein G may be coupled to the support, and then the bound protein used to bind antibodies or other binding proteins comprising a protein A or protein G binding portion. Likewise, avidin- or streptavidin-coated supports can be used for molecules that are biotinylated. Finally, polypeptide polypeptides can be engineered to have “tags” incorporated into the polypeptide, such as a His tag, and then use supports prepared with a molecule that binds the tag, such as nickel.

Many commercial kits are available for coupling, such as those from Life Sciences, InnovaBiosciences (Cambridge, UK), PlexBio (South San Francisco, Calif.), Polysciences, Inc. (Warrington, Pa.), and Bangs Laboratories, Inc. (Fishers, Ind.).

The size of the substrates can vary. For example, spherical supports can be 300 nm-200 μm or greater in diameter, including 300 nm, 400 nm, 500 nm, 600 nm, 700 nm, 800 nm, 900 nm, 1,000 nm (1 μm), 2 μm, 3 μm, 4 μm, 5 μm, 10 μm, 50 μm, 100 μm, 150 μm, and 200 μm or more.

Oligonucleotides can be attached to a solid support in a number of ways. Techniques for coupling nucleic acids to solid supports used to construct microarrays are well known in the art, including poly-L-lysine and phenylboronic acid methods. Most mRNAs contain a poly(A) tail at their 3′ end allowing them to be enriched by affinity chromatography, for example, using oligo(dT) or poly(U) coupled to a solid support, such as cellulose or Sephadex™ (see Ausubel et al., eds., 1994, Current Protocols in Molecular Biology, Vol. 2, Current Protocols Publishing, New York). Solid supports can be coated with a polymer such as polyethylene glycol (PEG) that does not comprise functional groups that interact with oligonucotides and their functional groups. PEG linkers of varying lengths can be used so that nucleic acids can be attached at varying distances from the solid support surface, thereby decreasing the amount of steric hindrance that may otherwise exist between nucleic acids and the complexes they ultimately form. The solid supports can be coated one or more times with a mixture of 2, 3, 4, or more PEG linkers of differing lengths. The end result is an increased distance between ends of PEG linkers attached to the solid support. Attachment of primers to the PEG linkers can be accomplished using any reactive groups known in the art. As an example, click chemistry can be used between azide groups on the ends of PEG linkers and alkyne groups on the primers.

Purification affinity tags can be used. Suitable affinity purification tags include members of binding partner pairs. For example, the tag may be a hapten or antigen, which will bind its binding partner. The binding partner can be attached to a solid support. For example, suitable binding partner pairs include antigens (such as proteins (including peptides)) and antibodies (including fragments thereof (Fabs, etc.)); proteins and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are also useful. Useful binding partner pairs include biotin (or imino-biotin) and streptavidin, digeoxeinin and Abs.

Additional techniques include enzymatic attachment, chemical attachment, photochemistry or thermal attachment and absorption.

Polymers having preferably more than one functional (or reactive) group can be used. Each of the functional groups is available for conjugation with a separate oligonucleotide. Useful polymers in this regard include those having hydroxyl groups, amine groups, thiol groups, and the like. Examples of suitable polymers include dextran and chitosan. Linear or branched forms of these polymers may be used. An example of a branched polymer with multiple functionalities is branched dextran. It will be apparent to those of ordinary skill in the art that any chimeric polymer or copolymer may also be used provided it has a sufficient number of functional groups for primer attachment.

Enzymatic techniques can be used to attach oligonucleotides to the support. For example, terminal transferase end-labeling techniques can be used (Hermanson, Bioconjugate Techniques, San Diego, Academic Press, 1996). A nucleotide labeled with a secondary label (e.g. a binding ligand, such as biotin) is added to a terminus of the oligonucleotide; supports coated or containing the binding partner (e.g. streptavidin) can thus be used to immobilize the target nucleic acid. Alternatively, the terminal transferase can be used to add nucleotides with special chemical functionalities that can be specifically coupled to a support. Similarly, random-primed labeling or nick-translation labeling (Hermanson, Bioconjugate Techniques, San Diego, Academic Press, 1996) can also be used. In some cases, the oligonucleotide is synthesized with biotinylated nucleotides or biotinylated after synthesis.

Chemical labeling (Hermanson, Bioconjugate Techniques, San Diego, Academic Press, 1996) can be used. Bisulfate-catalyzed transamination, sulfonation of cytosine residues, bromine activation of T, C and G bases, periodate oxidation of RNA or carbodiimide activation of 5′ phosphates can be done.

Photochemistry or heat-activated labeling can be done (Hermanson, Bioconjugate Techniques, San Diego, Academic Press, 1996). Thus for example, aryl azides and nitrenes preferably label adenosines, and to a less extent C and T (Aslam et al., Bioconjugation: Protein Coupling Techniques for Biomedical Sciences; New York, Grove's Dictionaries, 1998). Psoralen or angelicin compounds can also be used (Aslam et al., Bioconjugation: Protein Coupling Techniques for Biomedical Sciences; New York, Grove's Dictionaries, 1998). The preferential modification of guanine can be accomplished via intercalation of platinum complexes (Aslam et al., Bioconjugation: Protein Coupling Techniques for Biomedical Sciences; New York, Grove's Dictionaries, 1998).

The oligonucleotide can be absorbed on positively charged surfaces, such as an amine coated solid phase. The target nucleic acid can be cross-linked to the surface after physical absorption for increased retention (e.g. PEI coating and glutaraldehyde cross-linking; Aslam et al., Bioconjugation: Protein Coupling Techniques for Biomedical Sciences; New York, Grove's Dictionaries, 1998).

Direct chemical attachment or photocrosslinking can be done to attach the oligonucleotide to the solid phase, by using direct chemical groups on the solid phase substrate. For example, carbodiimide activation of 5′ phosphates, attachment to exocyclic amines on DNA bases, and psoralen can be attached to the solid phase for crosslinking to the DNA.

Argonaute:guide molecule complexes can be used to inhibit activity of target polynucleotide, such as for example targeting a splice site in an mRNA. By targeting an mRNA site, the Argonaute:guide molecule complex takes up residency at the splice site, inhibiting splicing complexes from interacting with the mRNA. Such inhibition may be in vivo (such as in a cell) or in vitro. Inhibition may be partial or complete.

### Argonaute:Guide Molecule Complexes as Probes

An embodiment is directed to using Argonaute:guide molecule complexes as probes in a sample.

Argonaute:guide molecule complexes can be detected in a variety of ways. For example, an affinity tag can be exploited such that a binding partner incorporates a detectable label. Suitable detectable labels include an enzyme, a radioisotope, a member of a specific binding pair; a fluorescent dye; a fluorescent protein; a quantum dot, and the like. Fluorescent labels of nucleotides include fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′ dimethylaminophenylazo) benzoic acid (DABCYL), Cascade dark Gray®, Oregon Green®, Texas Red®, Cyanine and 5-(2′-aminoethyl)aminonaphthalene-1-sulfonic acid (EDANS). When guide molecules are labeled, they can be advantageously labeled at the 3′ part of the molecule. Alternatively, an Argonaute:guide molecule complex can be detected by probing it, including probing any additional sequence beyond the recruiting and stabilization domains, with a suitable, detectably labeled probe. Detection can be done in vitro (such as in extracts) or in vivo (such as in a cell), in living or dead cells.

Alternatively, the guide molecules can be designed to be a molecular beacon. Molecular beacons are single-stranded oligonucleotide hybridization probes that form a stem-and-loop structure. The loop contains a probe sequence that is complementary to a target sequence, and the stem is formed by the annealing of complementary arm sequences that are located on either side of the probe sequence. A fluorophore is covalently linked to the end of one arm and a quencher is covalently linked to the end of the other arm. Molecular beacons do not fluoresce when they are free in solution. However, when they hybridize to a nucleic acid strand containing a target sequence they undergo a conformational change that enables them to fluoresce brightly.

The method of using such a beacon would comprise a detectable label that is at least one fluorophore, the fluorophore localized to the recruiting or stabilization domain forming a first arm, wherein the guide molecule comprises additional sequence at the 3′ end that is complementary to the domain comprising the at least one fluorophore, the sequence labeled with at least one quencher and forming a second arm; the first arm separated from the second arm by not more than about 60 nucleotides; the guide molecule forming with the target RNA or DNA sequence under detection conditions a double-stranded hybrid having a first strength; the first and second arm sequences having sufficient complementarity to one another to form under detection conditions a double-stranded stem hybrid having a second strength less than the first strength, whereby in the absence of the target RNA or DNA sequence fluorescence of the at least one fluorophore is quenched; and the first and second hybrid strengths being selected such that the guide molecule fluoresces when the at least one fluorophore is stimulated under detection conditions in the presence of the target RNA or DNA sequence. Compositions and methods detailing molecular probes and related technologies can be found in U.S. Pat. Nos. 5,925,517, 6,150,097, 6,037,130, 7,662,550, and 7,385,043.

The target nucleic acid can be RNA or DNA. The RNA or DNA can be a nuclear, mitochondrial, plastid (e.g., chloroplast), or a viral RNA or DNA.

In some cases, the target RNA or DNA encodes a disease or disorder-related marker, or an infectious agent marker. As a non-limiting example, Table 2 provides non-limiting example of tumor markers that can be detected with the methods of the invention. Infectious agents include cytomegalovirus, herpes simplex virus type I, II, H. pylori, ebolavirus, and varicella zoster virus.

TABLE 2
Tumor markers and associated tumor types
(Adapted from Casciato and Territo (Casciato and Territo, Manual of clinical oncology.
Lippincott Williams & Wilkins, Philadelphia, 2009))

| Tumor marker | Associated tumor types |
|---|---|
| Alpha fetoprotein (AFP) | germ cell tumor, hepatocellular carcinoma |
| Calretinin | mesothelioma, sex cord-gonadal stromal tumor, adrenocortical |
| | carcinoma, synovial sarcoma |
| Carcinoembryonic | gastrointestinal cancer, cervix cancer, lung cancer, ovarian cancer, |
| antigen | breast cancer, urinary tract cancer |
| CD34 | hemangiopericytoma/solitary fibrous tumor, pleomorphic lipoma, |
| | gastrointestinal stromal tumor, dermatofibrosarcoma protuberans |
| CD99MIC 2 | Ewing sarcoma, primitive neuroectodermal tumor, |
| | hemangiopericytoma/solitary fibrous tumor, synovial sarcoma, |
| | lymphoma, leukemia, sex cord-gonadal stromal tumor |
| CD117 | gastrointestinal stromal tumor, mastocytosis, seminoma |
| Chromogranin | neuroendocrine tumor |
| Cytokeratin (various | Many types of carcinoma, some types of sarcoma |
| types) | |
| Desmin | smooth muscle sarcoma, skeletal muscle sarcoma, endometrial |
| | stromal sarcoma |
| Epithelial membrane | many types of carcinoma, meningioma, some types of sarcoma |
| antigen (EMA) | |
| Factor VIII, CD31 FL1 | vascular sarcoma |
| Glial fibrillary acidic | glioma (astrocytoma, ependymoma) |
| protein (GFAP) | |
| Gross cystic disease | breast cancer, ovarian cancer, salivary gland cancer |
| fluid protein (GCDFP- | |
| 15) | |
| HMB-45 | melanoma, PEComa (for example angiomyolipoma), clear cell |
| | carcinoma, adrenocortical carcinoma |
| Human chorionic | gestational trophoblastic disease, germ cell tumor, choriocarcinoma |
| gonadotropin (hCG) | |
| immunoglobulin | lymphoma, leukemia |
| inhibin | sex cord-gonadal stromal tumor, adrenocortical carcinoma, |
| | hemangioblastoma |
| keratin (various types) | carcinoma, some types of sarcoma |
| lymphocyte marker | lymphoma, leukemia |
| (various types) | |
| MART-1 (Melan-A) | melanoma, steroid-producing tumors (adrenocortical carcinoma, |
| | gonadal tumor) |
| Myo D1 | rhabdomyosarcoma, small, round, blue cell tumor |
| muscle-specific actin | myosarcoma (leiomyosarcoma, rhabdomyosarcoma) |
| (MSA) | |
| neurofilament | neuroendocrine tumor, small-cell carcinoma of the lung |
| neuron-specific enolase | neuroendocrine tumor, small-cell carcinoma of the lung, breast cancer |
| (NSE) | |
| placental alkaline | seminoma, dysgerminoma, embryonal carcinoma |
| phosphatase (PLAP) | |
| prostate-specific antigen | prostate |
| PTPRC (CD45) | lymphoma, leukemia, histiocytic tumor |
| S100 protein | melanoma, sarcoma (neurosarcoma, lipoma, chondrosarcoma), |
| | astrocytoma, gastrointestinal stromal tumor, salivary gland cancer, |
| | some types of adenocarcinoma, histiocytic tumor (dendritic cell, |
| | macrophage) |
| smooth muscle actin | gastrointestinal stromal tumor, leiomyosarcoma, PEComa |
| (SMA) | |
| synaptophysin | neuroendocrine tumor |
| thyroglobulin | post-operative marker of thyroid cancer (but not in medullary thyroid |
| | cancer) |
| thyroid transcription | all types of thyroid cancer, lung cancer |
| factor-1 | |
| vimentin | sarcoma, renal cell carcinoma, endometrial cancer, lung carcinoma, |
| | lymphoma, leukemia, melanoma |

### Argonaute:Guide Molecule Complexes for Co-Purification and Depletion

Other embodiments are directed to a method of identifying an RNA binding polypeptide comprising binding to a target RNA sequence in an RNA molecule a complex comprising an Argonaute polypeptide and a heterologous, single-stranded oligonucleotide guide molecule that comprises a recruiting domain comprising at least 8 nucleotides at its 5′ end of the guide molecule (g1-g8) and a stabilization domain adjacent and 3′ to the recruiting domain and comprising at least 4 nucleotides (g9-g12) in a sample, wherein the stabilization domain of the guide molecule has sufficient complementarity to the target RNA sequence such that the Argonaute polypeptide:guide molecule complex binds stably to the target RNA sequence, isolating the Argonaute polypeptide:guide molecule complex bound to the target RNA sequence, and detecting polypeptides bound to the RNA molecule comprising the target RNA sequence. Such methods thus identify an RNA binding polypeptide by binding an Argonaute:guide molecule complex to a target RNA nucleic acid, isolating the bound RNA, and then isolating/analyzing those polypeptides bound to the RNA nucleic acid.

Examples of RNA-binding proteins are translation initiation factors that bind with messenger RNA (mRNA), small nuclear ribonucleoproteins (snRNPs), and RNA editing proteins such as RNA specific adenosine deaminase. These RNA binding proteins perform such functions as regulating translation and RNA splicing and editing.

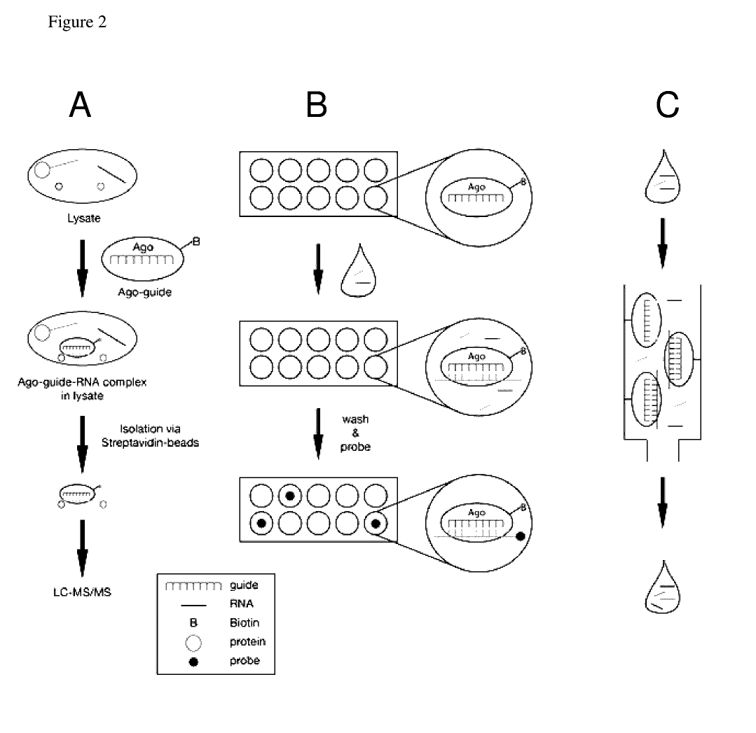

In the methods of the invention, the Argonaute:guide molecule complex acts as a means to target a specific RNA molecule by targeting a nucleic acid sequence within the specific RNA molecule. The Argonaute:guide molecule complex complex acts as means to purify targeted RNA and the molecules complexed with the RNA. For example and as shown in FIG. 2A, the Argonaute:guide molecule complex can be biotinylated, either on the guide molecule (on nucleotides that do not interfere with the guide molecule's targeting and stabilization functions), or preferably, on the Argonaute polypeptide. The biotinylated Argonaute:guide molecule complex is mixed with a cell lysate or other sample, and allowed to bind its target nucleic acid. A target nucleic acid comprises only a small portion of the target RNA molecule, but when the biotinylated Argonaute:guide molecule complex is isolated using avidin- or streptavidin-conjugated beads, the entire target RNA molecule will be isolated. The sample can be analyzed using liquid chromatography-tandem mass spectrometry to analyze the bound proteins.

As another example, before binding the Argonaute:guide molecule complex to its target RNA, proteins can be radiolabeled in a cell by supplying ³⁵S-methionine and/or ³⁵S-cysteine. Such labeled proteins can be visualized after isolating the Argonaute:guide molecule complex:target RNA complexes on SDS-PAGE gels and exposing the gel to X-ray film. The bound polypeptides can be purified from SDS-PAGE gels, end-sequenced or sequenced using mass spectrometry, and antibodies or nucleic acid probes made that can purify the bound polypeptides and its nucleic acid sequence, respectively.

In another embodiment, shown in FIG. 2B, Argonaute:guide molecule complexes can be targeted to RNA sequences that are specific to biomarkers or infectious agents; such complexes can be immobilized on a surface, such as in a microarray. A substrate is prepared with Argonaute:guide molecule complexes, immobilized by biotin (indicated by the letter “B” in FIG. 2B) to a streptavidin-coated surface. Samples are then applied, and the plate is probed for binding of the target to the Argonaute:guide molecule complex, visualized with a probe to the target sequence (filled circle in FIG. 2B).

Attached to a support, for example, Argonaute:guide molecule complexes can be used to purify from a sample, or deplete a sample of, a target polynucleotide (and molecules associated with the polynucleotide comprising the target polynucleotide, if desired) by contacting the sample with the support-Argonaute:guide molecule complex complexes (FIG. 2C). The support-Argonaute:guide molecule complex complexes can then be removed by centrifugation, gravity, magnetics (depending on the type of support used); or if the support is planar, the sample isolated from the support. Isolating the sample separate from the support-Argonaute:guide molecule complex complexes, thus depletes the sample of the target polynucleotide, such as a DNA sequence, or an RNA sequence, such as an rRNA or mRNA. In another embodiment, shown in FIG. 2C, Argonaute:guide molecule complexes can be used to purify a sample of one or more RNAs, such as rRNAs. A column is prepared with solid supports, to which are attached Argonaute:guide molecule complexes. A sample is applied to the column, and the target nucleic acids are bound by the Argonaute:guide molecule complexes; in this way, RNA molecules comprising the target nucleotide sequence(s) are removed from the sample. More than one type of Argonaute:guide molecule complex can be used simultaneously to remove multiple RNA sequences.

The same method can be used to purify target polynucleotides and associated molecules, such as RNA- and DNA-binding polypeptides. In such an instance, the support-Argonaute:guide molecule complex complexes would be saved, washed in appropriate buffers, and then either used in an application or the Argonaute:guide molecule complexes eluted from the support for further analysis or use.

### Argonaute:Guide Molecule Complexes as Designer Nucleic Acid Enzymes

Another embodiment is directed to a method of cleaving an RNA or DNA molecule, comprising binding to a target RNA or DNA sequence a complex comprising an Argonaute polypeptide and a heterologous, single-stranded oligonucleotide guide molecule that comprises a recruiting domain comprising at least 8 nucleotides at the 5′ end of the guide molecule (g1-g8) and a stabilization domain adjacent and 3′ to the recruiting domain and comprising at least 4 nucleotides (g9-g12) in a sample, wherein the stabilization domain of the guide molecule has sufficient complementarity to its target RNA or DNA sequence such that the Argonaute polypeptide:guide molecule complex binds stably to the target RNA or DNA sequence, and allowing the Argonaute polypeptide:guide molecule to cleave the RNA or DNA molecule.

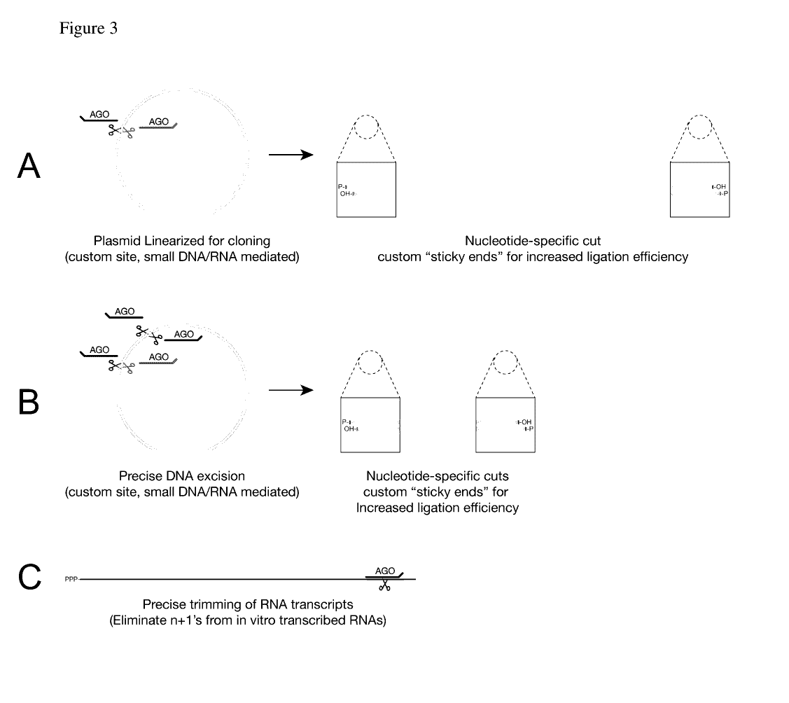

A further embodiment provides for methods to generate a double-stranded break in a double-stranded target nucleic acid using Argonaute:guide molecule complexes. An embodiment is directed to methods for generating a blunt end cut in a double-stranded target nucleic acid, as shown in FIG. 3A. A double-stranded target nucleic acid can be contacted with two Argonaute:guide molecule complexes. One Argonaute:guide molecule complex targets a region of a first strand of the double-stranded target nucleic acid. The other Argonaute:guide molecule complex targets a region of the second strand of the double-stranded target nucleic acid. In some instances the targeted region of the first strand of the double-stranded target nucleic acid and the targeted region of the second strand of the double-stranded target nucleic acid can overlap (e.g., be complementary) such that the cleavage by the Argonaute:guide molecule complex of each strand of the double-stranded target nucleic acid results in a blunt end, double-stranded break of the target nucleic acid. In other embodiments, single-stranded nucleic acids are cleaved, such as single-stranded RNA or DNA.

In some embodiments, the targeted regions of the first strand of the target nucleic acid and the second strand of the target nucleic acid may partially overlap, thereby promoting generation of sticky ends after cleavage. FIG. 3A depicts an exemplary embodiment of the generation of sticky ends by Argonaute:guide molecule complexes. A double-stranded target nucleic acid can be contacted with two Argonaute:guide molecule complexes. One Argonaute:guide molecule complex targets a region of a first strand of the double-stranded target nucleic acid. The other Argonaute:guide molecule complex targets a region of the second strand of the double-stranded target nucleic acid. A portion, or none, of the targeted region of the first strand of the double-stranded target nucleic acid and the targeted region of the second strand of the double-stranded target nucleic acid can be complementary to each other (e.g., overlap). The targeted region of the first strand of the double-stranded target nucleic acid and the targeted region of the second strand of the double-stranded target nucleic acid can partially overlap (e.g., be partially complementary) such that the cleavage by the Argonaute:guide molecule complexes of each strand of the double-stranded target nucleic acid results in a sticky end double-stranded break of the target nucleic acid.

In another embodiment and as shown in FIG. 3B, four Argonaute:guide molecule complexes can be used to liberate a fragment with sticky ends from a plasmid. One Argonaute:guide molecule complex targets a first region of a first strand of the double-stranded target nucleic acid. A second Argonaute:guide molecule complex targets a first region of the second strand of the double-stranded target nucleic acid. The targeted region of the first strand of the double-stranded target nucleic acid and the targeted region of the second strand of the double-stranded target nucleic acid can partially overlap (e.g., be partially complementary) such that the cleavage by the Argonaute:guide molecule complexes of each strand of the double-stranded target nucleic acid results in a sticky end double-stranded break of the target nucleic acid. A third Argonaute:guide molecule complex targets a second region of the first strand of the double-stranded target nucleic acid, and a fourth Argonaute:guide molecule complex targets the same region, of the second strand of the double-stranded target nucleic acid, as that of the third Argonaute:guide molecule complex. A portion, or none, of the second targeted region of the first strand of the double-stranded target nucleic acid and the second targeted region of the second strand of the double-stranded target nucleic acid can be complementary to each other (e.g., overlap). The targeted second region of the first strand of the double-stranded target nucleic acid and the targeted second region of the second strand of the double-stranded target nucleic acid can partially overlap (e.g., be partially complementary) such that the cleavage by the Argonaute:guide molecule complexes of each strand of the double-stranded target nucleic acid results in a sticky end double-stranded break of the target nucleic acid. In this manner, precise excision is possible. This same method also allows for the removal of a portion of DNA from a DNA molecule, thus preparing the DNA to receive an insert with complementary sticky ends, or to use the excised DNA as an insert for cloning.

These methods can easily be applied to single-stranded molecules, such as single-stranded RNA and DNA. In such instances, for each cleavage location, only one Argonaute:guide molecule complex is necessary.

For example, Argonaute:guide molecule complexes can be used to remove nucleotides that are added to RNA transcripts by RNA polymerase, referred to as “n+1” activity. Such activity can be detrimental in a number of applications. This application is shown in FIG. 3C, using a single Argonaute:guide molecule complex.

These methods can comprise contacting the target nucleic with a plurality of Argonaute:guide molecule complexes. A target nucleic acid can be contacted with at least about 1, 2, 3, 4, 5, 6, 7, 8, or 9 or more Argonaute:guide molecule complexes. The Argonautes of the Argonaute:guide molecule complexes may be the same or different.

While the guide molecules can be any length greater than 12 nucleotides that can be conveniently handled and maintain activity, guide molecules that are 12-15 nucleotides long are preferred. Guide molecules that are 12-15 nucleotides long have been found by the inventors, under appropriate conditions, to specifically cleave the intended target(s) with little, if any, production of mis-cleaved molecules (“side-products”). See the Examples.

The guide molecules of the Argonaute:guide molecule complexes may be the same or different. The guide molecules of the Argonaute:guide molecule complex (e.g., 2 Argonaute:guide molecule complexes) may differ by at least 1, 2, 3, 4, 5, 6, 7, 8, 9, or 10 or more nucleotides. The guide molecules of the Argonaute:guide molecule complexes may be fully or partially complementary to each other. The guide molecules may be complementary to each other over at least 1, 2, 3, 4, 5, 6, 7, 8, 9, 10, 11, 12, 13, 14, 15, 16, 17, 18, 19, 20, or 21 or more consecutive nucleotides. Nucleic acid-targeting nucleic acids can be fully or partially complementary to each other when they are designed to target overlapping regions on each strand of a double-stranded target nucleic acid.

The Argonaute proteins may be the same or different Argonaute proteins. When the two Argonaute proteins are different, they may differ by at least 1, 2, 3, 4, 5, 6, 7, 8, 9, 10, 20, 30, 40, 50, 60, 70, 80, 90 or 100%. Argonaute may differ in the N, PAZ, MID, and/or PIWI domain.

The selection of buffers in which the Argonaute:guide molecule complexes cleave their targets is critical to obtain complete cleavage over time without the production of side-products. Generally, the buffers comprise buffer that maintains the target pH of the reaction. Such buffers can be selected by one of skill in the art. Examples of buffers include N-(2-acetamido)-2-aminoethanesulfonic acid (ACES), N-(2-acetamido)iminodiacetic acid (ADA), N,N-bis(2-hydroxyethyl)-2-aminoethanesulfonic acid (BES), 2-(N-morpholino)ethanesulfonic acid (MES), 3-(N-morpholino)-propanesulfonic acid (MOPS), 3-(N-morpholinyl)-2-hydroxypropanesulfonic acid (MOPSO), piperazine-N,N′-bis(2-ethanesulfonic acid) [Pipes], N-tris-(hyrdroxymethyl)-methyl-2-aminoethanesulfonic acid (TES), 3-[N-tris (hydroxymethyl) methylamino]-2-hydroxypropanesulfonic acid (TAPSO), and 3-[N-tris-(hydroxymethyl-mettlylamino]-propanesulfonic acid (TAPS); a preferred buffer is 2-[4-(2-hydroxyethyl)piperazin-1-yl]ethanesulfonic acid (HEPES), such as 2-[4-(2-hydroxyethyl)piperazin-1-yl]ethanesulfonic acid (HEPES)-KOH and HEPES-NaOH. A preferred concentration of the buffer is about 18 mM, although other concentrations can be used from about 1 mM to about 200 mM, including 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 50 mM, 75 mM, 100 mM, 125 mM, 150 mM, 175 mM, and 200 mM. Suitable pH values for cleaving reactions are those of about pH 7 to about pH 8.8, including pHs of about 7, 7.1, 7.2, 7.3, 7.4, 7.5, 7.6, 7.7, 7.8, 7.9, 8.0, 8.1, 8.2, 8.3, 8.4, 8.5, 8.6, 8.7, and about 8.8, with about pH 7.4 to about pH 7.5 being preferred.

In preferred embodiments, the buffer comprises a salt, such as potassium chloride (KCl), sodium chloride (NaCl) or monosodium glutamate (C₅H₈NNaO₄) at suitable concentrations. Concentrations of these salts that are useful range from about 25 mM to about 100 mM, such as (in mM) 25, 30, 35, 40, 45, 50, 55, 60, 65, 70, 75, 80, 85, 90, 95, and 100. A preferred concentration of sodium chloride is 50 mM, a preferred concentration of monosodium glutamate is 75 mM, and a preferred concentration of potassium chloride is 100 mM. Buffers can also further comprise metal cations, such as divalent metal cations, such as Mn²⁺ and Mg²⁺. In a preferred embodiment, the metal cations are supplied by MnCl₂ salt. Metal cation salts, such as MnCl₂ and MgCl₂, are present at suitable concentrations, such as from 1 mM to about 100 mM, such as 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 50 mM, 75 mM, and 100 mM; in a preferred embodiment, MnCl₂ salt is present at a concentration of about 3 mM. The buffer can further comprise a reducing agent, such as dithiothreitol (DTT) or 2-mercaptoethanol (β-mercaptoethanol). These reagents are present at a suitable concentration from about 1 mM to about 20 mM, such as 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, and 20 mM. In a preferred embodiment, the reducing agent is DTT at a concentration of about 5 mM.

The buffer can further comprise a detergent, such as a nonionic, non-denaturing detergent or a zwitterionic nondenaturing detergent. Examples of nonionic, non-denaturing detergents include poly(ethyleneoxy)ethanol (IGEPAL®-CA630, Nonidet™ P-40); Octylphenolpoly(ethyleneglycolether)ₓ (Triton® X-100), Polyethylene glycol tert-octylphenyl ether (Triton® X-114), Polyoxyethylene (23) lauryl ether (Brij® 35), Polyethylene glycol hexadecyl ether (Brij® 58), Polyethylene glycol sorbitan monolaurate (Tween® 20), Polyethylene glycol sorbitan monooleate (Tween® 80), and octylglucoside. In a preferred embodiment, the detergent is poly(ethyleneoxy)ethanol. Examples of zwitterionic nondenaturing detergents include 3-((3-cholamidopropyl) dimethylammonio)-1-propanesulfonate (CHAPS) and 3-([3-Cholamidopropyl]dimethylammonio)-2-hydroxy-1-propanesulfonate (CHAPSO). Detergents may be present at a concentration of 0.001% to about 2%, including (in %) 0.001, 0.005, 0.01, 0.025, 0.05, 0.075, 0.1, 0.125, 0.150, 0.175, 0.2, 0.225, 0.250, 0.275, 0.3, 0.325, 0.350, 0.375, 0.4, 0.425, 0.450, 0.475, 0.5, 0.525, 0.550, 0.575, 0.6, 0.65, 0.7, 0.75, 0.8, 0.85, 0.9, 0.95, 1, 1.25, 1.5, 1.75, and 2.

Buffers can also comprise additional agents, such as glycerol or sugars (such as sucrose). In this embodiment, the glycerol or sugar is present at about 1-20% (w/v), including about (in % (w/v)) 1, 2, 3, 4, 5, 6, 7, 8, 9, 10, 11, 12, 13, 14, 15, 16, 17, 18, 19, and about 20.

In some embodiments, buffers are prepared two-fold to about five-fold concentrated.

### Argonaute:Guide Molecule Complexes to Facilitate Cloning

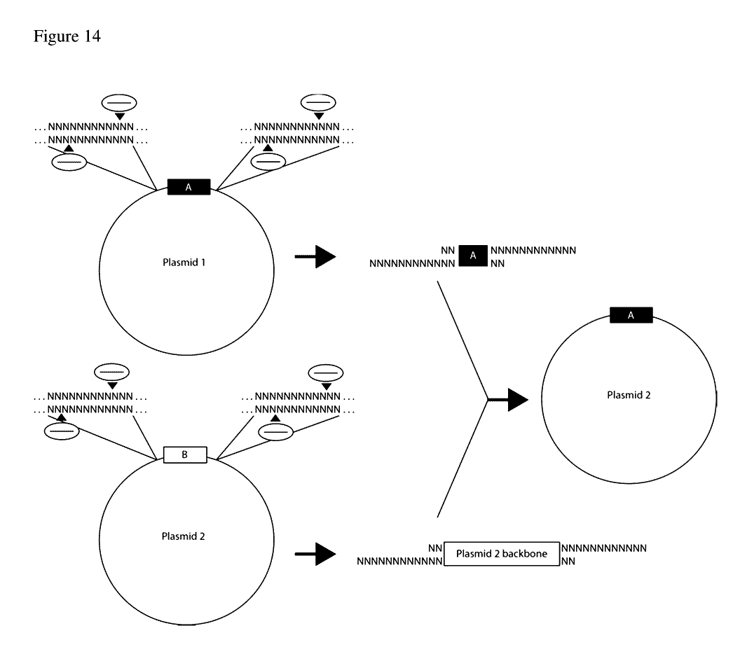

Argonaute:guide molecule complexes can be used to subclone double-stranded nucleic acid fragments. For example, where a desired fragment is to be subcloned into a plasmid, complimentary sticky-ends can be generated as described previously in the plasmid and in liberating the desired fragment from its host DNA. The prepared fragment and prepared plasmid are isolated and combined in the presence of a ligase to complete the subcloning. The use of sticky ends allow for directional cloning of the desired fragment. This procedure is summarized in FIG. 14 and described in the Examples.

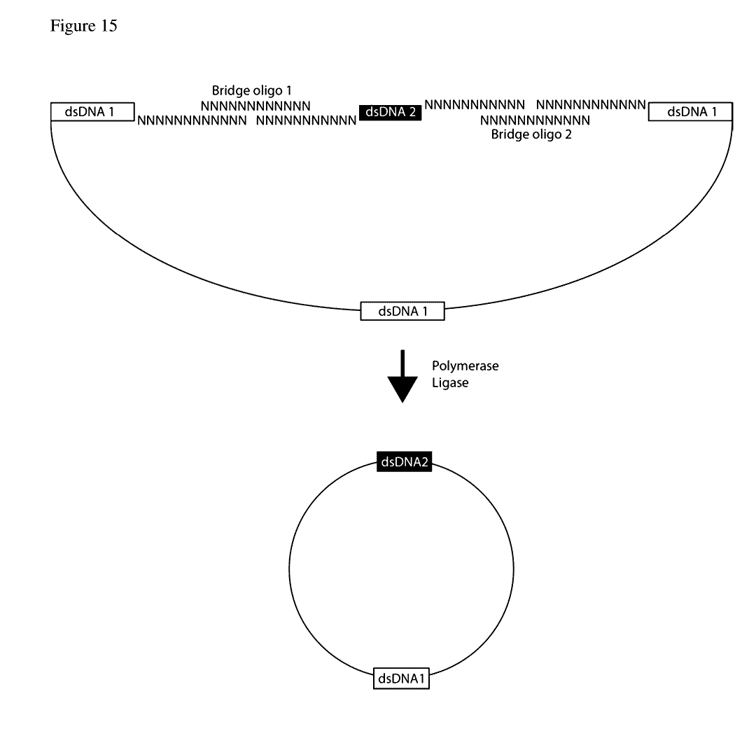

In an example where a desired fragment is to be cloned into another double stranded DNA molecule, but where complementary 3′ and 5′ overhangs cannot be generated between the donor and acceptor molecules, bridging oligonucleotides can be used to bridge the overhangs of the desired fragment into the target DNA molecule. Sticky ends are generated using the methods as described above, and two bridge oligonucleotides that are complementary to overhangs of the 3′ and 5′ ends of the dsDNA1 and dsDNA2 (FIG. 15) are hybridized, and the complex incubated with polymerase and then ligase to complete the subcloning (FIG. 15, bottom). The use of bridge oligonucleotides also permits for directional cloning. See the Examples.

In another approach, fragments of double-stranded DNA molecules generated by Argonaute:guide molecule complexes are cloned without ligase. In this embodiment, guide molecules are designed to be complementary to regions of the double stranded target to produce longer sticky ends. Because these sticky ends are longer, when they hybridize to their complementary sequence, they are sufficiently stably complexed, thus completing the cloning, and are transformed into a host cell without further ligase treatment. One skilled in the art can determine the optimal length for the sticky ends depending on factors such as the characteristics of the targeted overhang sequences themselves (e.g., repeats), GC content, melting point, and annealing temperature. In some embodiments, the sticky ends are from 18 to 24 nucleotides long or longer; such as 18, 19, 20, 21, 22, 23, or 24 nucleotides long or longer.

### Kits

An embodiment is directed to a kit, comprising an Argonaute polypeptide and a single-stranded oligonucleotide guide molecule that comprises a recruiting domain comprising 8 nucleotides at the 5′ end of the guide molecule (g1-g8) and a stabilization domain adjacent and 3′ to the recruiting domain and comprising at least 4 nucleotides (g9-g12) and having a sequence sufficiently complementary to a target RNA or DNA molecule nucleic acid sequence such that the Argonaute polypeptide:guide molecule complex binds stably to the target RNA or DNA sequence.

(a) Containers or Vessels

Reagents included in kits can be supplied in containers of any sort such that the life of the different components are preserved and are not adsorbed or altered by the materials of the container. For example, sealed glass ampules may contain lyophilized components (such as guide strand molecules), or buffers that have been packaged under a neutral, non-reacting gas, such as nitrogen. Containers may also contain Argonaute polypeptides. Suitable buffers include those that permit an Argonaute polypeptide and guide molecule to complex, and/or to bind their target RNA or DNA molecules. Ampules may consist of any suitable material, such as glass, organic polymers (i.e., polycarbonate, polystyrene, etc.), ceramic, metal or any other material typically employed to hold reagents. Other examples of suitable containers include simple bottles that may be fabricated from similar substances as ampules, and envelopes that may have foil-lined interiors, such as aluminum or alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate and/or may be supplied as an electronic-readable medium, such as a floppy disc, CD-ROM, DVD-ROM, DVD, videotape, audio tape, etc. Detailed instructions may not be physically associated with the kit; instead, a user may be directed to an internet web site specified by the manufacturer or distributor of the kit, or supplied as electronic mail.

In one embodiment, kits of the invention include an Argonaute polypeptide and a single-stranded nucleic acid guide molecule. In some embodiments, the kits provide for Argonaute:guide molecule complexes that bind disease or disorder marker sequence, or an infectious agent sequence. The guide molecules and/or the Argonaute polypeptides may be supplied on solid supports. The Argonaute:guide molecule complexes may further be labeled with a detectable label.

In one embodiment, a kit comprises Argonaute protein packaged in a buffer, such as 2× or 5× buffer. Suitable buffers include those described for Argonaute:guide molecule complex-mediated nucleic acid cleavage. A preferable buffer (at 1×) comprises 18 mM HEPES-KOH, pH 7.4; 50 mM NaCl, 3 mM MnCl₂, 0.01% octylphenoxy poly(ethyleneoxy)ethanol, 5 mM DTT, and 10% glycerol. Another preferable buffer comprises 18 mM HEPES-KOH, pH 7.4; 75 mM C₅H₈NNaO₄, 3 mM MnCl₂, 0.01% octylphenoxy poly(ethyleneoxy)ethanol, 5 mM DTT, and 10% glycerol. Such a kit can be used to facilitate cloning, biological sample component identification (such as non-biologic and biologic samples, including hematopoietic and non-hematopoietic samples) for identifying biomarkers, viruses, mRNA or isoforms; precipitation of RNA and RNA-protein complexes, and for histological component identification, such as identifying unique markers, genes, mutations, and aneuploidy. Steps in using the kit comprise mixing desired guide molecules with the Argonaute protein, heating for a period of time, and then incubating the resulting Argonaute:guide molecule complexes with the sample to be analyzed (or the nucleic acid to be cloned, or the nucleic acid/protein complexes to be purified from). In further embodiments, the kit comprises instructions for the intended use.

In another embodiment, a kit comprises a multiwall plate to which surface is bound Argonaute protein. The surface of the multiwall plate can be coated with Argonaute protein by using SNAP (protein tag derived from the human DNA repair protein O6-alkylguanine-DNA alkyltransferase and acts irreversibly on O6-benzylguanine (BG) derivatives) or other protein tags, (strept)avidin-biotin linkages, etc. The wells of the plates can be filled with a buffer. A preferable buffer (at 1×) comprises 18 mM HEPES-KOH, pH 7.4; 50 mM NaCl, 3 mM MnCl₂, 0.01% octylphenoxy poly(ethyleneoxy)ethanol, 5 mM DTT, and 10% glycerol. Another preferable buffer comprises 18 mM HEPES-KOH, pH 7.4; 75 mM C₅H₈NNaO₄, 3 mM MnCl₂, 0.01% octylphenoxy poly(ethyleneoxy)ethanol, 5 mM DTT, and 10% glycerol. Such a kit is useful for regular and high-throughput screening (HTS) for identifying, for example, nucleic acid components within a sample and for quantifying nucleic acid components within a sample. In an embodiment, the kit is used by adding desired guide molecules to the plates with the bound Argonaute and heating the mixture to form Argonaute:guide molecule complexes. Samples are then added to the wells, incubated, and washed. The wells are then imaged for signal using, for example, hybrid capture assays, branched DNA (bDNA) assays, padlock probes, multiplexing (as available from NanoString, for example), and DNA/RNA binding protein with a detectable label, such as a fluorescent dye. In further embodiments, the kit comprises instructions for use.

In yet another embodiment, the kit comprises pre-packaged Argonaute:guide molecule complex with pre-determined guide sequences. Such a kit can be used, for example, as a diagnostic tool, singe molecule microsocopy, live cell imaging, etc. The kit can further comprise instructions for use.

### EXAMPLES

The following examples are given to aid in the understanding of the invention, not to limit it in any way.

### Example 1

### Colocalization Single Molecules Spectroscopy (CoSMoS)

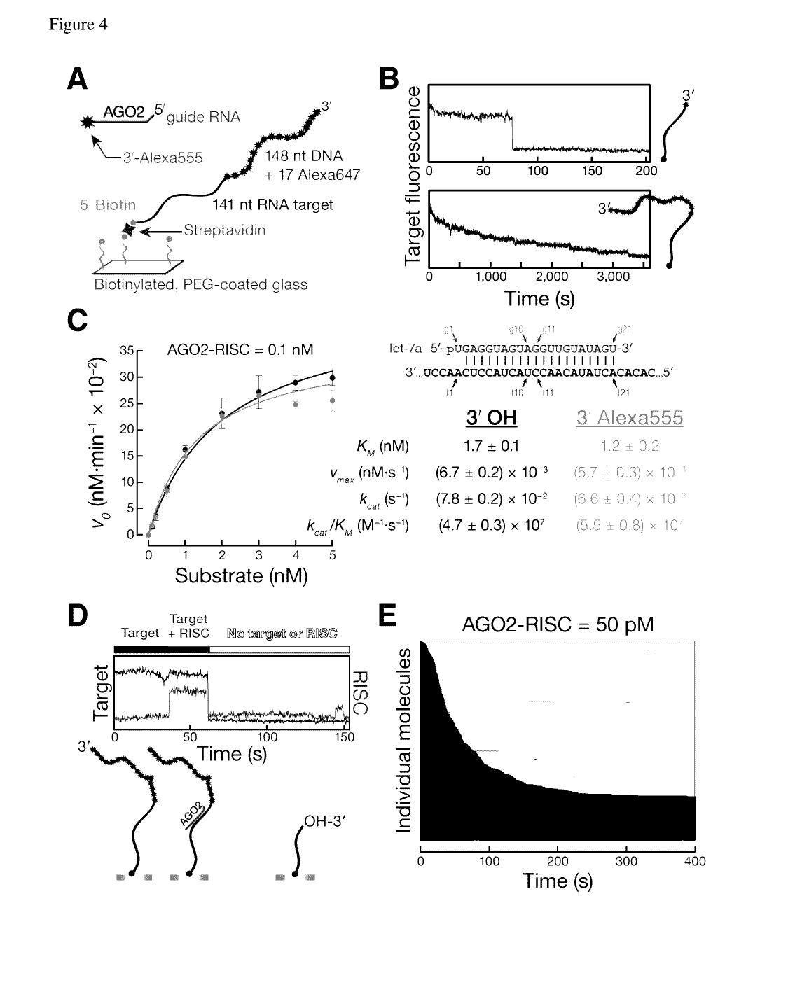

To measure how Argonaute proteins alter the properties of nucleic acid oligonucleotides, we used Colocalization Single Molecules Spectroscopy (CoSMoS), an implementation of multicolor total internal reflection fluorescence (TIRF) microscopy that achieves high signal-to-noise ratios by exciting only those fluorescent molecules immediately above the slide surface (Friedman et al. Biophys J 91, 1023-1031, 2006). To adapt CoSMoS to study RISC, a fluorescently labeled target RNA was attached to a glass surface via a biotin-streptavidin-biotin-PEG 3,400 linkage and then incubated with purified RISC assembled in vitro to contain a fluorescent guide strand (FIG. 4A). The strategy relies on two novel reagents developed for these studies: (1) a target RNA designed to allow the unambiguous differentiation between target cleavage and photobleaching; and (2) RISC assembled via the cellular Argonaute-loading pathway using a siRNA containing a fluorescently labeled guide strand and then purified to remove contaminating free guide (Flores-Jasso et al., RNA 19, 271-279, 2013).

Photobleaching of fluorescent molecules is a technical challenge that plagues many single-molecule experiments, especially when high time resolution is required or when a molecule of interest must be continuously excited with laser light for an extended time. To overcome photobleaching and to distinguish photobleaching from target cleavage, we constructed a 141 nucleotide RNA target containing 17 Alexa647 dyes within a 148 nucleotide DNA 3′ extension. This multiply labeled target provided two related advantages. First, its extreme brightness allowed the use of decreased laser power, thereby decreasing the rate at which individual dyes photobleached. This allowed long observation times (30 min continuous illumination capturing 10,000 frames at 100 ms per frame; FIG. 4B). Second, the presence of multiple Alexa647 dyes yielded a characteristic stepwise photobleaching pattern that was readily distinguishable from the all-or-none fluorescence change caused by target cleavage and 3′ product release. FIG. 4B compares two molecules undergoing photobleaching: the target labeled with a single 3′ Alexa647 dye undergoes binary signal loss indistinguishable from target cleavage, whereas the target bearing 17 Alexa647 groups gradually loses fluorescence in multiple discrete steps.

Mouse Argonaute 2 (AGO2) was loaded with an RNA guide in extract from Ago2^{-/-} mouse embryonic fibroblasts overexpressing AGO2 under the control of the murine stem cell virus promoter (O'Carroll et al., Genes Dev 21, 1999-2004, 2007). Loading was accomplished using a double-stranded siRNA carrying a 3′ Alexa555 group on the guide strand; programmed RISC was then sequence-affinity purified (Flores-Jasso et al., RNA 19, 271-279, 2013). To test whether dye addition altered the properties of AGO2-RISC, we compared the KM and kcat of AGO2 programmed with an unmodified guide corresponding to the sequence of let-7a to AGO2 programmed with the 3′ Alexa555-labeled guide (FIG. 4C). The KM for let-7a-loaded RISC (1.7±0.1 nM) was nearly identical to that containing 3′ Alexa555-labeled let-7a (1.2±0.2 nM). Moreover, these KM values agree well with previous values for human AGO2-RISC (mouse AGO2 is 99% similar to human AGO2, differing only in seven N-terminal amino acids; Martinez and Tuschl, Genes Dev 18, 975-980, 2004; Rivas et al., Nat Struct Mol Biol 12, 340-349, 2005; Ameres et al., Cell 130, 101-112, 2007). Similarly, kcat for RISC containing the 3′ Alexa555-labeled let-7a guide (6.6±0.4×10-² sec-¹) was similar to the kcat of RISC programmed with let-7a without the dye (kcat=7.8±0.2×10-² sec-¹).

### Example 2

### RISC Changes the Rate—Determining Step for Nucleic Acid Hybridization

Argonaute proteins have been proposed to increase the rate of nucleic acid hybridization by pre-organizing the nucleotides of the seed sequence into a stacked conformation that makes productive collisions with target more likely. The association rate constant, kon, for mammalian AGO2 has been inferred from KD and koff values measured in ensemble binding experiments (Wee et al., Cell 151, 1055-1067, 2012) or estimated by fitting pre-steady state ensemble data to a three-phase exponential model in which the fastest phase was assumed to correspond to kon (Deerberg et al., Proc Natl Acad Sci USA 110, 17850-17855, 2013).

To measure kon directly, we simultaneously recorded the fluorescence of individual target RNA attached to the slide and individual molecules of mouse AGO2-RISC containing fluorescent guide strand (FIG. 4D). For each target RNA molecule, RISC arrival time was taken to be the first detectable co-localization of RISC fluorescence and target RNA fluorescence. We restricted that the arrivals of RISC molecules must remain co-localized with a target ≧400 msec (i.e., two frames at 5 frames·−s-¹). FIG. 4D provides an example of Alexa555-labeled RISC arriving at an Alexa647-labeled target: when RISC arrives at ˜40 s, the Alexa 555 fluorescence co-localizing with the Alexa647 target increases in a single step; it remains high until both Alexa555 (RISC) and ALexa647 (target) fluorescence drop to baseline at ˜60 s, signifying target cleavage and simultaneous RISC and 3′ product release. FIG. 4E displays 426 individual single-molecule traces, ordered by time of target cleavage, as a ‘rastergram.’ Rastergrams summarize the arrivals, departures, and target cleavage events for many individual target molecules.

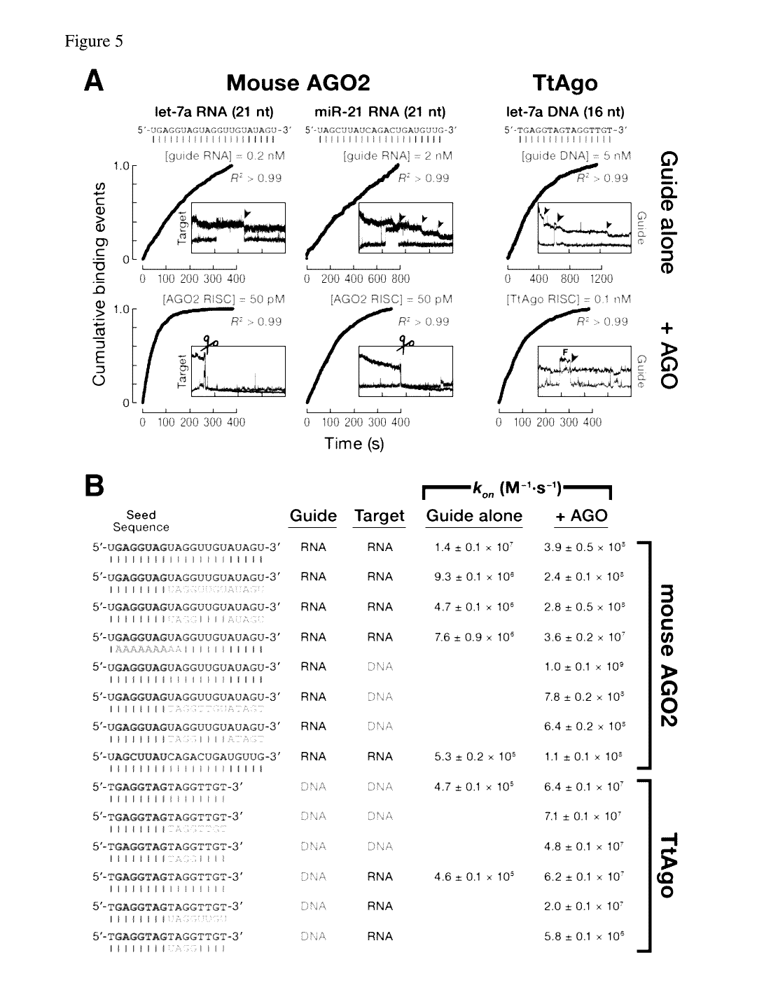

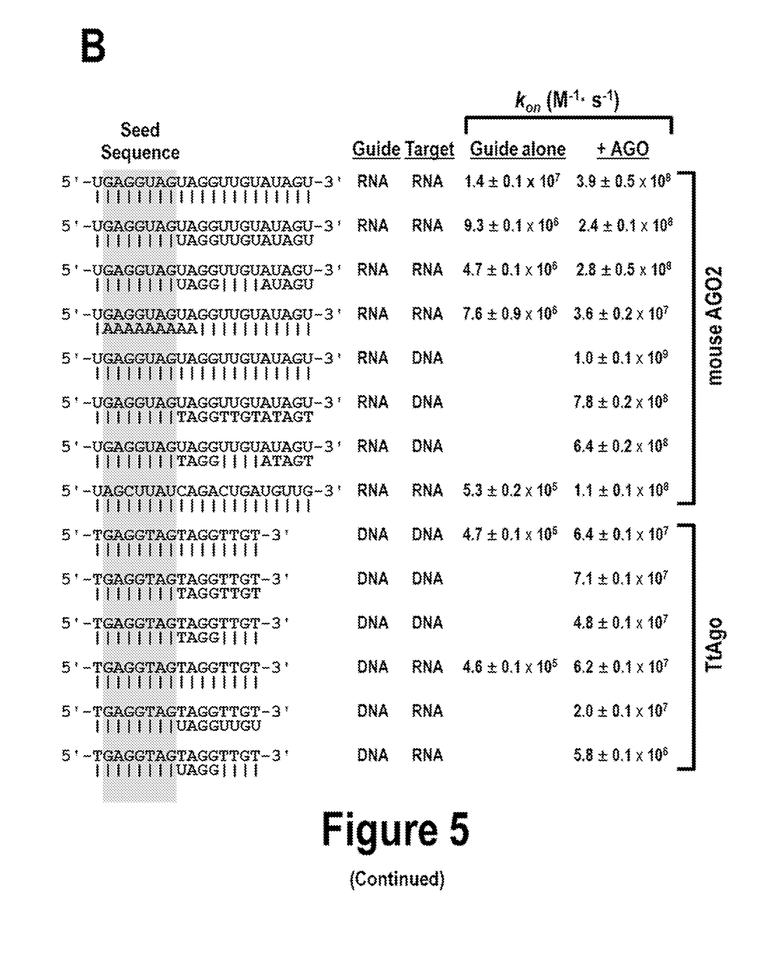

To understand how AGO2 changes the rate at which an oligonucleotide arrives at a target, we used CoSMoS to compare the kon of single-stranded let-7a RNA and let-7a-programmed AGO2-RISC (FIG. 5). After their arrival at the target, let-7a alone and let-7a bound to AGO2 follow different paths. Formation of a 21 bp RNA:RNA duplex is essentially irreversible under physiological conditions, so observation of let-7a ended when its Alexa555 label photobleached, and, subsequently, no new, fluorescent RISC was detected binding the target; the target, labeled with 17 Alexa647 dyes continued to be detectable, gradually losing fluorescence via discrete photobleaching events (FIG. 5A). In contrast, binding of let-7a RISC ended with target cleavage; Alexa555 and Alexa647 fluorescence were lost simultaneously.

On-rates for RISC (kon) or let-7a alone were determined by fitting the cumulative distribution of arrivals to a single exponential, corrected for nonspecific background binding to the slide. The on-rate of let-7a RNA alone binding to a fully complementary target (1.4±0.1×10⁷ M-¹·−s-¹; FIG. 5A) was considerably slower than the rate of macromolecular diffusion. The sequence of let-7a comprises only three (A, G, and U) of the four nucleotides. The on-rate for let-7a alone measured by CoSMoS agrees well with previous single-molecule estimates of kon for short oligonucleotides lacking G or C (Zhang et al., eLife 3, e01775, 2014). In comparison, kon for let-7a-programmed mouse AGO2-RISC binding to the fully complementary target RNA (3.9±0.5×10⁸ M-¹·s-¹) was ˜25-fold faster than kon for let-7a alone. Moreover, the rate at which AGO2-RISC finds its target approaches the limit of macromolecular diffusion at 37° C.: ˜6.4×10⁹ M-¹˜•s-¹ under our standard conditions of 20% glycerol (Segur and Oberstar, Industrial and Engineering Chemistry 43, 2117-2120, 1951; Berg and von Hippel, Annu Rev Biophys Biophys Chem 14, 131-160, 1985). Thus, as proposed previously (Wee et al., Cell 151, 1055-1067, 2012; Deerberg et al., Proc Natl Acad Sci USA 110, 17850-17855, 2013; Jung et al., J Am Chem Soc 135, 16865-16871, 2013), Argonaute accelerates productive arrival of its guide at a complementary target sequence.

While these data indicate that AGO2 improves kon by ˜25-fold for binding of let-7a to a fully complementary target sequence, the unusual sequence composition of let-7a might understate the general enhancement in target finding. To test this, we determined kon for miR-21, a miRNA containing all four nucleotides, either alone or bound to mouse AGO2 (FIG. 5). As expected from its greater sequence complexity, miR-21 RNA alone bound its complementary RNA target ˜26 times more slowly (kon, miR-21 alone=5.3±0.2×10⁵ M-¹·−s-¹) than did let-7a alone. But mouse AGO2 accelerated miR-21 binding ˜200-fold (kon miR-21 AGO2-RISC=1.1±0.1×10⁸ M-¹·−s-¹). Thus, accelerating oligonucleotide target finding to close to the rate of diffusion is a general property of AGO2-RISC.

Is this acceleration of target finding a general property of other Argonaute proteins or unique to mouse AGO2? To answer this, we measured kon to a fully complementary sequence for TtAgo, the DNA-guided Argonaute protein (Swarts et al., Nature 507, 258-261, 2014) from the eubacterium Thermus thermophilus. T. thermophilus grows optimally at 70° C. (Cava et al., Extremophiles 13, 213-231, 2009), and TtAgo does not efficiently cleave either RNA or DNA at 37° C. Control experiments established that the addition of an Alexa555 dye to the 3′ end of the DNA guide does not alter the ensemble binding properties of TtAgo. In vivo, TtAgo binds 16 nucleotide DNA guides, so we loaded TtAgo at 70° C. with a single-stranded DNA comprising the first 16 nucleotide of let-7a, and then studied its binding at 37° C. using CoSMoS. On its own, the 16 nucleotide “let-7a” guide bound a complementary RNA target ˜130 times more slowly (kon=4.7±0.1×10⁵M-¹·−s-¹) than the same DNA guide incorporated into TtAgo (kon=6.4±0.1×10⁷M-¹·−s-¹; FIG. 5). Thus, both mouse AGO2 and TtAgo, despite >2.5 billion years of evolutionary divergence, retain the ability to alter the rate-determining step for nucleic acid hybridization (kon) so that the speed at which Argonaute finds its complementary target RNA or DNA is limited by the rate of macromolecular diffusion.

### Example 3

### Argonaute Accelerates the Rate of Target Finding by Creating the Seed Sequence

The three structural domains of Argonaute proteins divide their guide RNAs into discrete functional domains. To determine which of these functional domains contributes most to the Argonaute-dependent enhancement of target binding, we measured kon using three different target RNAs: (1) a target complementary just to the seed sequence (g2-g8); (2) a target complementary to both the seed and the region of 3′ supplementary pairing (g13-g16); and (3) a target with complete complementarity to the guide (g2-g21; FIG. 2B). For each target RNA, we determined kon for both the guide alone and the guide loaded into mouse AGO2 (FIG. 5). We also measured kon for let-7a binding to a non-target having ≦6 nucleotide complementary to any region of the let-7a guide sequence and ≦4 nucleotide complementary to the let-7a seed sequence. For this essentially non-complementary control RNA, we were unable to detect any binding interactions above non-specific background binding to the slide.

Structural comparisons of eubacterial and human AGO2 show that an N-terminal Argonaute domain prevents pairing beyond g16 in animal Argonautes; and computational analyses of piRNAs in flies, silk moths, and mice suggest that target cleavage does not require complementarity beyond target position t16 (Wang et al., Nature 461, 754-761, 2009; Kwak and Tomari, Nat Struct Mol Biol 19, 145-151, 2012; Wee et al., Cell 151, 1055-1067, 2012; Faehnle et al., Cell Rep 3, 1901-1909, 2013; Hauptmann et al., Nat Struct Mol Biol 20, 814-817, 2013; Wang et al., Molecular Cell 56:708-716, 2014). Thus even for targets with complete complementarity to the guide strand, they are unlikely to pair past g16 when the guide is bound to Argonaute.

In the absence of protein, nucleic acid hybridization is favored by greater complementarity, presumably because the larger number of potential base pairs provides more opportunities for nucleation, the rate-determining step for productive binding (Egli and Saenger, Principles of Nucleic Acid Structure (Springer Advanced Texts in Chemistry) Springer, 1988). Consistent with this principle, kon for let-7a RNA alone increased >10-fold, from 9.3±0.1×10⁶_{M}-1ₛ-1 for the target with complementarity only to the seed sequence (g1-g8) or 4.7±0.1×10⁶_{M}-1ₛ-1 for the target with seed and 3′ supplementary pairing (g1-g8 plus g13-g16) to 1.4±0.1×10⁷_{M}-1ₛ-1 for the fully complementary target (g1-g21) FIG. 5B). Yet when loaded in AGO2-RISC, let-7a bound all three targets with very similar, near diffusion-limited on-rates (varying from 2.4±0.1×10⁸ M-¹·−s-¹ to 3.9±0.5×10⁸ M-¹·−^{s}-¹; FIG. 5B). In contrast, the apparent rate of target finding for an RNA fully complementary to let-7a except for the seed nucleotides (3.6±0.2×10⁷) was ˜10-fold slower (FIG. 5B). We conclude that the seed sequence created by mouse AGO2 accounts for most of the enhancement in the rate of target finding.

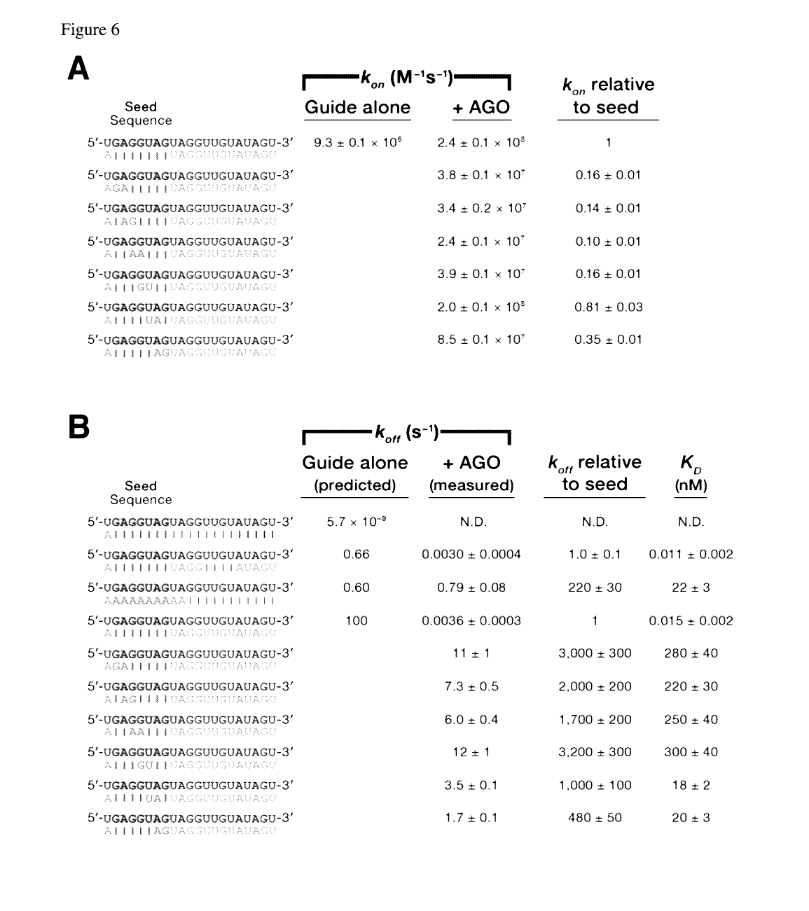

To further test this idea, we measured kon for a series of six target RNAs with a dinucleotide mismatch to the seed-pairing sequence (FIG. 6A). For these experiments, we required detection of an arriving RISC in at least two frames at 10 frames·−s-¹. Compared to a seed-matched target, dinucleotide mismatches with guide positions g2g3, g3g4, g4g5, or g5g6 reduced kon 6.3- to 10-fold. Mismatches with g6g7 and g7g8 reduced kon 1.2- to 2.9-fold, compared to a target complementary with the entire seed sequence. These data further support the view that Argonautes accelerate target finding by pre-organizing the seed, and the acceleration is diminished when the seed pairing is disrupted. They also suggest that positions g2-g5 of the seed contribute more to a successful encounter of RISC with target RNA than positions g6 or g7.

TtAgo also required seed complementarity to accelerate the rate of target finding: the on-rate for TtAgo bound to a 16 nucleotide DNA guide complementary to the target DNA, in only the seed (kon=7.1±0.1×10⁷ M-¹·−s-¹) or in both the seed and the 3′ supplementary region (kon=4.8±0.1×10⁷ M-¹·−s-¹), was essentially the same as when the entire guide was complementary to the target (kon=6.4±0.1×10⁷ M-¹·−s-¹); FIG. 5, Table 3).

TABLE 3
Binding properties of Argonaute-bound guide complexes with target RNAs or DNAs
with different extents of complementarity to the guide strand
(N.D., not determined because koff was much slower than the photobleaching rate)

| Extent of | RNA Target | | | DNA Target | | |
|---|---|---|---|---|---|---|
| complementarity | ko1, (M⁻¹ · s⁻¹) | koff (s⁻¹) | KD (nM) | ko1, (M⁻¹ · s⁻¹) | koff (s⁻¹) | KD (nM) |

21 nucleotide let-7a RNA loaded in mouse AGO2

| | | | | | | |
|---|---|---|---|---|---|---|
| Complete | 3.9 ± 0.5 × 10⁸ | N.D. | N.D. | 1.0 ± 0.1 × 10⁹ | N.D. | N.D. |
| Seed only | 2.4 ± 0.1 × 10⁸ | 0.0036 ± 0.0003 | 0.015 ± 0.002 | 7.8 ± 0.2 × 10⁸ | 0.41 ± 0.09 | 0.53 ± 0.07 |
| Seed + 3′ | 2.8 ± 0.5 × 10⁸ | 0.0030 ± 0.0004 | 0.011 ± 0.002 | 6.4 ± 0.2 × 10⁸ | 0.57 ± 0.02 | 0.88 ± 0.12 |
| Supplementary | | | | | | |

16 nucleotide let-7a-derived DNA guide loaded in TtAgo

| | | | | | | |
|---|---|---|---|---|---|---|
| Complete | 6.2 ± 0.1 × 10⁷ | N.D. | N.D. | 6.4 ± 0.1 × 10⁷ | N.D. | N.D. |
| Seed only | 2.0 ± 0.1 × 10⁷ | 0.35 ± 0.03 | 17 ± 2 | 7.1 ± 0.1 × 10⁷ | 1.0 ± 0.1 | 15 ± 2 |
| Seed + 3′ | 5.8 ± 0.1 × 10⁶ | 0.51 ± 0.06 | 88 ± 12 | 4.8 ± 0.1 × 10⁷ | 0.86 ± 0.08 | 18 ± 2 |
| Supplementary | | | | | | |

### Example 4

### Seed Mismatches Cause Rapid Dissociation of Mouse AGO2 RISC

Ensemble experiments at 25° C. show that mouse AGO2-RISC departs slowly from seed-matched targets (˜2,000 s at 25° C.; Wee et al., Cell 151, 1055-1067, 2012), a time-scale too long for direct observation by fluorescence of individual RISC molecules, because photobleaching of the guide RNA dye generally occurs before a departure is observed (the rate of Alexa555 photobleaching in our experiments was ˜0.06 s-¹, τ˜17 s). As an alternative strategy to measure koff for mouse AGO2-RISC at 37° C., a more physiologically appropriate temperature, we measured the apparent koff over a range of laser exposure (i.e., by changing the frame length) and extrapolated to no laser exposure (the y-intercept) to obtain koff: 0.0036±0.0003 s-¹, ˜280 s. In contrast, because the photobleaching rate was much slower than the dissociation rate, koff was readily measured by standard methods for the six targets containing a dinucleotide mismatch within the let-7a seed-match (FIG. 6B). Compared to the seed-matched target, RISC dissociated from these seed-mismatched targets from 480 to 3,200 times faster. As we observed for kon, individual positions within the seed contributed differentially to anchoring AGO2-RISC on its target RNA, with base pairs at g2-g6 contributing more than base pairs at g7 or g8. Thus, RISC discriminates between seed-matched and seed-mismatched targets both during its initial search and after it has bound; it finds seed-mismatched targets more slowly and remains bound to them for less time than seed-matched targets.

### Example 5

### Mouse AGO2, but not TtAgo, Discriminates Between RNA and DNA Targets

Mouse AGO2, like all known animal Argonautes, has only been reported to function by binding RNA targets. In contrast, TtAgo can cleave both RNA and DNA targets, although only DNA targets have been identified in vivo (Wang et al., Nature 456, 921-926, 2008; Wang et al., Nature 456, 209-213, 2008; Wang et al., Nature 461, 754-761, 2009; Swarts et al., Nature 507, 258-261, 2014). How do animal Argonaute proteins discriminate between RNA and DNA? We compared the binding of mouse AGO2 to RNA targets with binding to the same sequences composed of DNA (FIG. 5B). As we observed for RNA, AGO2 accelerated the search for a complementary binding site on DNA. In fact, kon for a seed-matched (g2-g8), seed-matched plus supplementary pairing (g2-g8, g13-g16), or a completely complementary (g2-g16) DNA target was ˜2.3 to 3.3 times faster than the on-rate for the corresponding RNA target (Table 3). However, mouse AGO2-RISC did not remain stably bound to the DNA, dissociating, on average, after just ˜2.4 s (koff=0.41±0.09 s-¹) after binding a seed-matched DNA target. In contrast, AGO2-RISC remained bound to an otherwise identical RNA target for an average of ˜280 s (koff=0.0036±0.0003 s-¹; Table 3). We observed a similar difference in koff for DNA and RNA targets with both seed and 3′ supplementary complementarity. The >110-fold faster dissociation of AGO2-RISC from DNA compared to RNA supports the view that even when acting in the nucleus, eukaryotic RISCs bind nascent transcripts, not single-stranded DNA (Buhler et al., Cell 125, 873-886, 2006; Sabin et al., Mol Cell 49, 783-794, 2013).

In contrast to mammalian Argonautes, bacterial Argonautes are thought to preferentially bind and cleave foreign DNA, such as horizontally transferred plasmids (Olovnikov et al., Mol Cell 51, 594-605, 2013; Swarts et al., Nature 507, 258-261, 2014). Consistent with this function, TtAgo showed no substantive binding preference for RNA over DNA targets (FIG. 5B and Table 3). TtAgo found its binding sites in RNA and DNA at similar rates (e.g., seed-matched target kon=2.0±0.1×10⁷ for RNA versus 7.1±0.1×10⁷ for DNA; FIG. 5B), and, once bound, departed from RNA and DNA at similar rates (koff=0.35±0.03 s-¹ for RNA versus 1.0±0.1 s-1 for DNA; Table 3).

### Example 6

### A Kinetic Framework for Mammalian RNAi

With a 5′-tethered target, target cleavage by RISC leaves the 5′ cleavage product tethered to the slide surface, allowing detection of RISC that remains bound via the guide nucleotides g11-g21. When let-7a guides mouse AGO2, target cleavage and release of the 5′ cleavage product from RISC were simultaneous within the time resolution of our experiments (e.g., FIG. 4D). This suggests that 5′ product release is faster than RISC dissociation from the 3′ cleavage product that contains the seed complementary sequence.

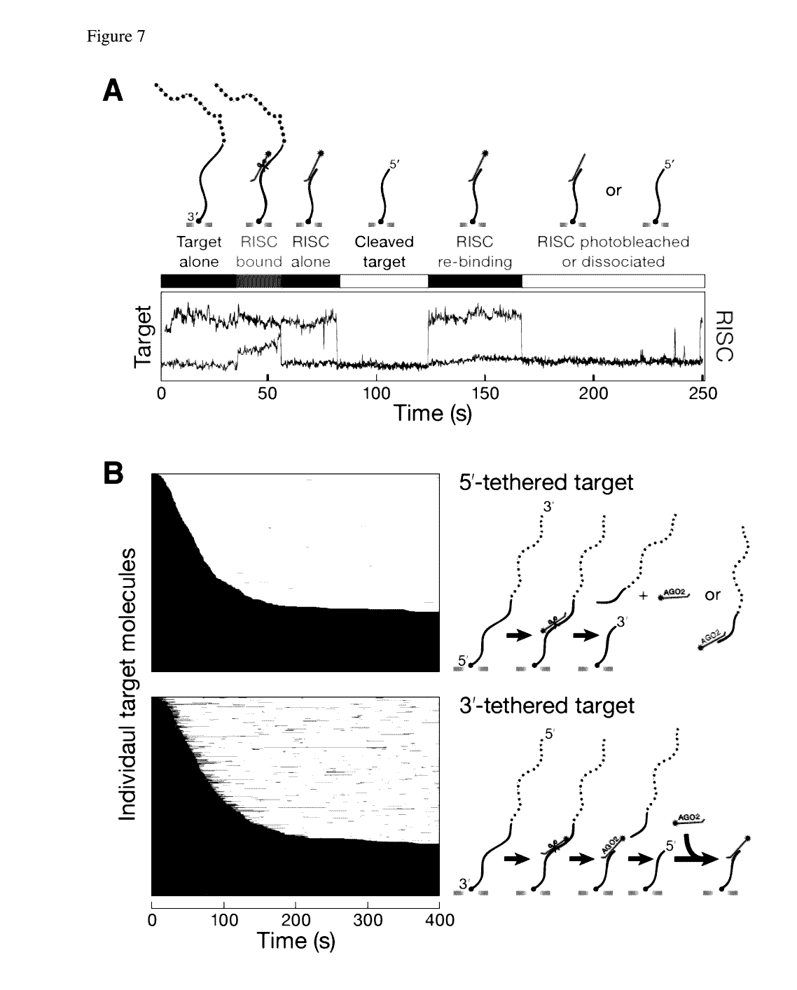

To directly measure 3′ cleavage product release, we synthesized a let-7a-complementary target with biotin on its 3′ end and 16 Alexa647 dyes at the 5′ end. Together, the 3′-tethered target allowed us to detect four distinct reaction species: (1) target alone, (2) RISC bound to the target, (3) RISC bound to the 3′ cleavage product, and (4) the 3′ product after RISC dissociation (FIG. 7A). The previous experiments with the 5′-tethered target adds information about RISC bound to 5′ cleavage product and the 5′ product alone, completing the set of all observable species in the RNAi reaction. FIG. 7B presents rastergrams that summarize observations of hundreds of RNA target molecules where the reaction states of AGO2-RISC and target were observed for the entire duration of the experiment.

As expected, both 3′- and 5′-tethered targets produced nearly identical AGO2-RISC binding rates. The kon for AGO2-RISC binding to the 3′-tethered target was 3.7±0.1×10⁸ M-¹·−s-¹, agreeing well with that for the 5′-tethered target (kon=3.9±0.5×10⁸ M-¹·−s-¹, FIG. 7B). With regard to product release, however, we observed striking differences in the order and rates of dissociation rates of the 5′ and 3′ cleavage products (FIG. 7). The first product to be released was nearly always the 5′ product, after which AGO2-RISC slowly dissociated from the 3′ product. After AGO2-RISC departure, we frequently observed its rebinding to the 3′ cleavage product (FIG. 7B). The 3′ product is complementary to the seed sequence; thus, let-7a AGO2-RISC maintains high affinity for a seed-match even after target cleavage, highlighting the essential role of the seed in RISC binding.

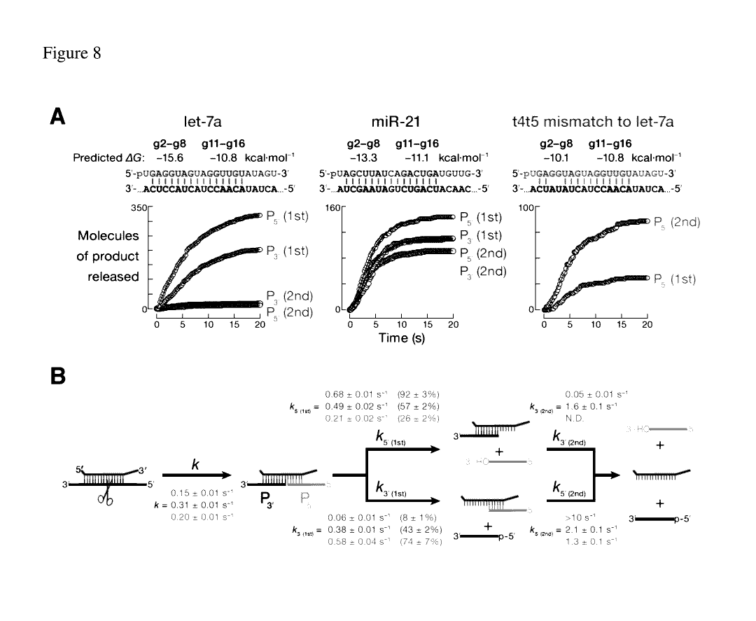

To quantitatively assess the product release mechanism, we performed global fitting (FIG. 5A) of a unified reaction scheme that accounts for all observed intermediates and products in the RNAi reaction (FIG. 8B). This reaction mechanism includes branched pathways for product release: one branch corresponds to the 5′ product being released first and the 3′ product released subsequently (FIG. 8B, k5′ 1st followed by k3′ 2nd), while in the other the order of product release is reversed (FIG. 8B, k3′ 1st followed by k5′ 2nd). Both branches arrive at the same final state: two free products and free AGO2-RISC. The inclusion of an additional step was required to account for the sigmoidal kinetics of product release (FIG. 8A). The rate constant for this additional step likely corresponds to the rate constant (k) of the slowest step in the target cleavage reaction—e.g., the conformational change in Argonaute that brings the catalytic Mg²⁺ near the scissile phosphate—rather than the actual chemical step of slicing. The global fit based on four experimental product release curves obtained from experiments with 5′- and 3′-tethered targets defined three of the five rate constants (k, k5′ 1st and k3′ 1st). The rate constants (k3′ 2nd and k5′ 2nd) for release of the 3′ or 5′ products following release of the other product were determined directly from the distributions of waiting times from the departure of the first cleavage product to the departure of the second, after subtracting the photobleaching rate.

### Example 7

### Seed Pairing Determines the Rate of Slicing and the Order of Product Release

In order to determine whether the features of cleavage and product release observed for let-7a RISC depend on the guide RNA identity and base-pairing stability with the target, we performed experiments paralleling those shown in FIG. 7B with 5′- and 3′-tethered targets fully complementary to miR-21 miRNA and AGO2-RISC loaded with miR-21. We also made a 5′-tethered let-7a target that contained mismatches with let-7a seed at positions g4 and g5. We then carried out global fitting of the kinetic scheme in FIG. 8B to these data to determine slicing and product release rates, as well as the order of product release.

We find that the slicing rate depends on guide strand identity: let-7a has the slowest slicing rate we measured (k=0.15 s-¹), while miR-21 AGO2-RISC cleaves its target twice as fast (k=0.31 s-¹). The let-7a has the strongest seed pairing with G=−15.6 kcal·mol-¹. The miR-21 seed is significantly weaker at G=−13.3 kcal·mol-¹. The let-7a target with seed mismatches that weaken the seed base pairing by about 5 kcal·−mol-¹ (G=−10.1 kcal·mol-¹) is cleaved faster than perfectly complementary let-7a target (k=−0.20 s-¹), but still slower than miR-21. This trend appears to inversely correlate with the stability of seed pairing to the target, albeit not precisely, indicating that there may be additional determinants of the cleavage rate. Interestingly, miR-21 AGO2-RISC failed to cleave or even detectably bind the miR-21 target with g4g5 mismatches, indicating that weakening seed base pairing below a certain threshold abolishes target recognition and cleavage.

The stability of seed pairing with respect to the stability of base pairs in the 3′-part of the guide strand determines the order of product release, as clearly evidenced by the proportion of the reaction directed through one of the two product release branches (FIG. 8B, values in parentheses). For let-7a, whose seed pairing (g2-g8; G=−15.6 kcal·mol-¹) is predicted to be more stable than the pairing of the 3′-half of the guide with the 5′ product (g11-g16; G=−10.8 kcal·mol-¹), the 5′ product was released before the seed-matching 3′ cleavage product for 92% of molecules.

For let-7a, release of the 3′ cleavage product from RISC limits the rate of enzyme turnover, kcat. Is 3′ product release generally rate-determining for RISC turnover? To test this, we analyzed two additional miRNA:target combinations: miR-21 paired to a fully complementary target and let-7a paired to a target bearing two mismatches to the let-7 seed. Unlike let-7a, miR-21 has a smaller predicted difference in the stabilities of target pairing to the seed and 3′-half (Gg2-g8=−13.3 kcal·mol-¹ versus Gg11-g16=−11.1 kcal·mol-¹). For miR-21, only 57% of the 5′ product was released first. For let-7a paired to a target mismatched to guide positions g4g5, the 5′ product is predicted to pair with let-7a more stably than the partially seed-matching 3′ product (Gg2-g8=−10.1 kcal·mol-¹ versus Gg11-g16=−10.8 kcal·mol-¹). For this guide:target pair, only 26% of the 5′ product was released before the 3′ cleavage product. We conclude that the order of product release—5′ first or 3′ first—does not follow a strict order but rather reflects the sequences of the guide and target.

Product release rates followed the base pair stability trends. For the 3′ product, the release rate of let-7a paired to a fully complementary target (0.06 s-¹) was tenfold slower than for let-7a paired to the g4g5:t4t5 mismatched target differ by an order of magnitude (0.6 s-¹), reflecting their different predicted free energies of base pairing between the seed sequence and the 3′ product (−15.6 kcal·mol-¹ versus −10.1 kcal·mol-¹). For the 5′ product, the release rates were more similar—0.7 s-¹ for let-7a, 0.5 s-¹ for miR-21, and 0.2 s-¹ for let-7a with the seed-mismatched target. The free energies of pairing for all three 5′ products were roughly −11 kcal·mol-¹.

### Example 8

### Release of the First Product Promotes Release of the Second

Global fitting analysis provides two rates for the release of each product, a rate for when the product departs first, leaving the other product still bound and a rate for when the product departs second with the other product having already dissociated. For example, k5′ 1st is the rate for 5′ product release in the presence of bound 3′ product, while k5′ 2nd is the rate for 5′ product release after the 3′ product departure. Our data suggest that release of the first product promotes release of the second product (FIG. 5). For example, the rates of the 5′ and 3′ products of miR-21 were both ˜4-fold faster when they were released second rather than first. Similarly, release of the 5′ product of cleavage of the let-7a seed-mismatched target was 0.21±0.01 s-¹ when released first, but 1.3±0.1 s-¹ when released second. A notable exception was the seed-matched 3′ product of let-7a target, by far the most stably bound product we examined. This 3′ cleavage product dissociated at ˜0.05 s-¹ regardless of the presence of the 5′ product.

We can imagine two mechanisms by which the departure of one product can accelerate dissociation of the other. Their binding may be mutually stabilized by stacking interactions between the terminal bases of the products. Supporting this view, the 5′ product of miR-21 leaves ˜4-fold faster when it departs after the 3′ product (and vice versa)—a ˜0.9 kcal·mol-¹ difference in stability. This difference in G is within the range of the contributions of dangling nucleotides to RNA helix stability (Xia et al., Thermodynamics of RNA Secondary Structure Formation. In RNA, Soll, D., S. Nishimura, and P. B. Moore, eds. (Oxford: Pergamon), pp. 21-48, 2001). Alternatively, departure of one of the two products may facilitate a conformational change that destabilizes the second product. Such a conformational change might correspond to the return of the endonuclease active site to the conformation present prior to zippering of the guide:target helix 3′ to the seed sequence (Wang et al., Nature 461, 754-761, 2009; Elkayam et al., Cell 150, 100-110, 2012; Schirle and MacRae, Science 336, 1037-1040, 2012; Faehnle et al., Cell Rep 3, 1901-1909, 2013).

### Example 9

### Strong Seed Pairing Slows RISC Turnover

The rates for target cleavage and product release allow calculation of the overall turnover rate: kcat=k·−k5′ 1st·−k3′ 2nd/(k·−k5′ 1st+k·−k3′ 2nd+k5′ 1st·−k3′ 2nd) when the 5′ product is released first, and kcat=k·−k3′ 1st·−k5′ 2nd/(k·−k3′ 1st+k·−k5′ 2nd+k3′ 1st·−k5′ 2nd) when the 3′ product is released first. Both pathways result in similar kcat values, due to the same trends of product release rates in both pathways for the two products. For let-7a, the calculated kcat value, 0.036±0.002 s-¹, agrees well with kcat determined by traditional initial velocity analysis (0.066±0.004 s-¹; FIG. 4C). The calculated turnover rate was about fourfold faster for both miR-21 (0.16±0.1 s-¹) and let-7a with the g4g5 seed-mismatched target (0.13±0.1 s-¹). The slower kcat for let-7a reflects the stronger seed pairing to its fully complementary target (G=−15.6 kcal·mol-¹, k3′ 1st=0.06 s-¹, k3′ 2nd=0.05 s-¹). This slow 3′ product release step for let-7a limits the overall turnover rate. Both miR-21, with its weaker pairing strength in its seed, and let-7a paired to the g4g5 seed-mismatched target whose seed pairing was intentionally weakened with mismatches, direct faster cleavage than let-7a with a fully complementary target, because their product release rates are comparable to or faster than k, the apparent RISC cleavage rate (FIG. 8B). Thus, guide RNAs, including siRNAs, with more stable base pairing between their seed and their target are predicted to cleave fewer targets per unit time than targets whose rate of 3′ product release is not rate-determining.

### Example 10

### AGO2 Distinguishes Between miRNA—Like Binding Sites and the Products of Target Cleavage

The free energy of base pairing between the seed sequence and its target influences the rates of all steps in the RNAi reaction, including binding and dissociation of RISC, cleavage of the target, and release of the cleaved products. However, one aspect of RISC function emerges from our studies that is not predicted by the stability of guide-target base pairing: AGO2 appears to discriminate between a miRNA-like binding site, which typically pairs only with nucleotides g2-g8, and binding to the seed-matched, 3′ product of target cleavage, which pairs with nucleotides g2-g10 (FIG. 6).

For let-7a, the predicted AG for g2-g10 base pairing with the 3′ product of target cleavage (ΔGg2-g10) is −16.9 kcal·mol-¹; the predicted ΔG for g2-g8 seed pairing to a full-length target RNA (ΔGg2-g8) is −13.3 kcal·mol-¹. Yet the rate of dissociation from AGO2 for the 3′ product (0.05 s-¹) was tenfold faster than for the seed-only target (0.0036 s-¹). Thus, AGO2 discriminates between seed pairing and product binding. When catalyzing RNAi, AGO2 acts as a classical, multiple turnover enzyme: its affinity for the product of cleavage is lower than for its substrate, despite the structural and energetic similarities between substrate and product. When acting as an RNA-guided, RNA-binding protein—the typical function of RISC guided by miRNAs—AGO2 remains bound, on average, for ˜280 s (for seed-match target) rather than 20 s (for 3′ product).

How can AGO2 bind more weakly to the 3′ product of target cleavage than to the seed-matched target, an RNA with which it makes two fewer base pairs? We hypothesize that extending base pairing beyond position g8 of the seed may trigger a conformational rearrangement in RISC that accelerates dissociation of the 3′ product. Such a conformational switch would explain why base pairing beyond guide position g8 is not typical for miRNA-target interactions. Alternatively, AGO2 might make sequence-independent, stabilizing contacts with the RNA backbone of the seed-matched target. Of course, such contacts are not possible with the 3′ cleavage product.

### Example 11

### Site-Specific Cleavage of Double-Stranded DNA with TtAgo

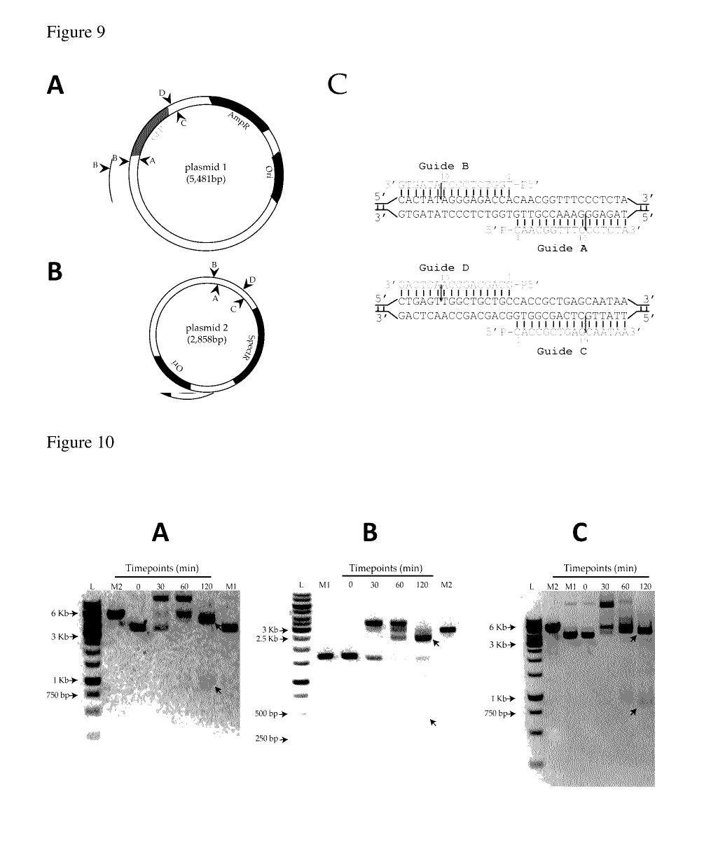

For each double-stranded cut, a pair of DNA guides was designed to be complementary to the target site. All Argonaute proteins, including TtAgo, always cleave the phosphodiester bond linking target nucleotides t10 and t11, the nucleotides that pair with guide nucleotides g10 and g11. Shown in FIG. 9A is the 5481 bp plasmid pET GFP LIC cloning vector (u-msfGFP) (Addgene plasmid #29772; hereafter designated plasmid 1), which confers ampicillin resistance and contains the green fluorescent protein gene, which was cleaved using two pairs of guides (shown in FIG. 9C) predicted to generate 980 bp and 4501 bp double-stranded DNA cleavage products. Shown in FIG. 9B is the 2858 bp plasmid pET empty polycistronic destination vector (2Z) (Addgene plasmid #29776; hereafter designated plasmid 2), which confers spectinomycin resistance, was cleaved using the same pair of guides (FIG. 9C) predicted to generate 278 bp and 2580 bp double-stranded DNA cleavage products.

Cleavage reactions containing TtAgo, guided by a pair of 16 nucleotide single-stranded DNA guides as shown in FIG. 9C, and double-stranded plasmid target DNA were incubated for the indicated times in FIG. 10 in buffer 1 or buffer 2 (see Experimental Procedures Example). FIG. 10A shows cleavage of plasmid 1 (5481 bp; FIG. 9A) in buffer 1 using the guides described in FIG. 9C, yielded the expected 980 bp and 4501 bp double-stranded DNA products. FIG. 10B shows cleavage of plasmid 2 (2858 bp; FIG. 9B) in buffer 1 using the same guides described in FIG. 9C similarly yielded the expected 278 bp and 2580 bp double-stranded DNA products. FIG. 10C shows cleavage of plasmid 1 (5481 bp; FIG. 9A) with buffer 2 using the guides described in FIG. 9C, yielded the expected 980 bp and 4501 bp double-stranded DNA products.

This data demonstrates buffer 1 is active on more than one plasmid for the generation of expected products, and that buffer 2 is also effective to that end.

### Example 12

### Guide Length Variation for Site-Specific Cleavage of Double-Stranded DNA with TtAgo

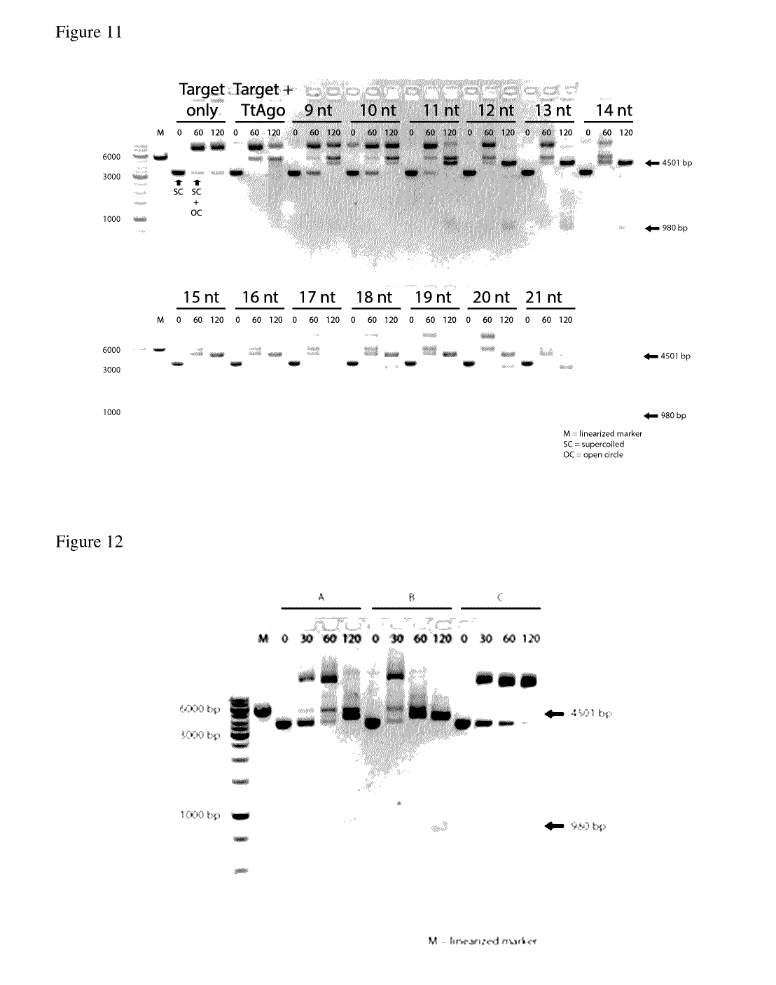

Controls including both target only and target with TtAgo (no guides) and cleavage reactions containing TtAgo, guided by two pair of small, single-stranded DNA ranging in length from 9 nucleotides to 21 nucleotides and double-stranded plasmid 1 target DNA were incubated for the indicated times in FIG. 11 (minutes) in buffer 1. As shown in FIG. 11, incubating TtAgo with single-strand DNA guides ranging from 9 to 21 nucleotides in length all yielded the desired cleavage products of 980 bp and 4501 bp. All starting plasmid was consumed after 2 hours with guides ≧12 nucleotides. TtAgo loaded with guides 12 to 15 nucleotides demonstrate complete cleavage to desired products with minimal additional products formed. Guides 16 nucleotides to 21 nucleotides show predicted cleavage products and increasing amounts of additional products.

The data suggest an optimal guide length for cleavage occurs over a narrow range, namely 12-15 nucleotides. The data also indicates that the extent of additional target nucleotides complementarity to the stabilization region of the guide beyond 15 nucleotides is correlated to increasing off-target activity.

### Example 13

### Cleavage Enablement by Buffers 1 and 2 Compared to Published Buffer for the Site-Specific Cleavage of Double-Stranded DNA with TtAgo

Cleavage reactions containing TtAgo, guided by two pairs of 12 or 21 nucleotide single-stranded DNA guides, and double-stranded plasmid 1 target DNA were incubated for the indicated times (minutes; FIG. 12) in buffers 1 or 2 or a buffer comprised of 10 mM Tris-HCl pH 8, 125 mM NaCl and 0.5 mM MnCl₂ (Swarts et al., 2014. DNA-guided DNA interference by a prokaryotic Argonaute. Nature, 507, 258-261) hereafter referred to as buffer 3. FIG. 12A shows cleavage of plasmid 1 (5481 bp; FIG. 9A) in buffer 1 using 12 nucleotide guides generates the expected 980 bp and 4501 bp double-stranded DNA products. FIG. 12 B shows cleavage of plasmid 1 (5481 bp; FIG. 9A) in buffer 2 using 12 nucleotide guides generates the expected 980 bp and 4501 bp double-stranded DNA products. FIG. 12C shows cleavage of plasmid 1 (5481 bp; FIG. 9A) in buffer 3 using 21 nucleotide guides generates predominately an open circle form of the target DNA plasmid and a minor linearized form.

This data demonstrates that buffer 3 was unconducive to cleavage, but that the two buffers (buffer 1 and buffer 2) presented here can generate the expected products in a specific fashion.

### Example 14

### Effects of Dinucleotide Mismatches on the Site-Specific Cleavage of Double-Stranded DNA with TtAgo

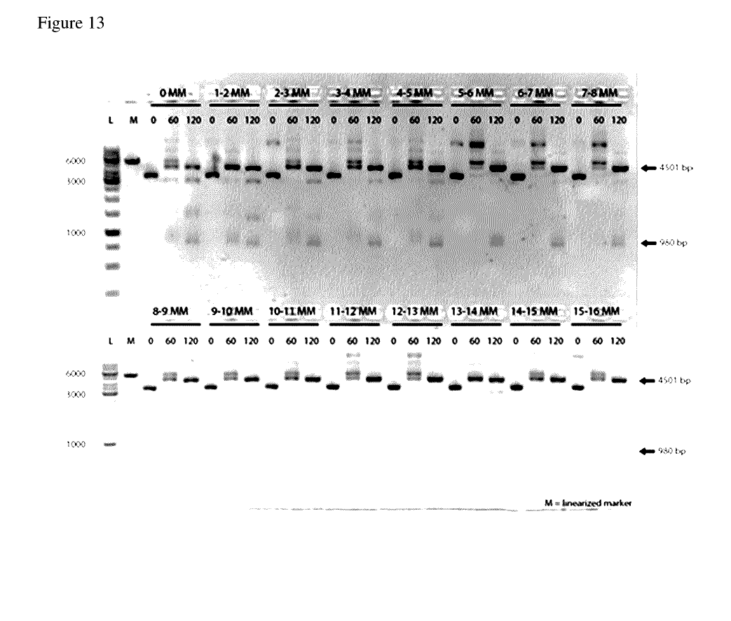

Cleavage reactions containing TtAgo, guided by two pairs of 16 nucleotide single-stranded DNA guides as described in FIG. 9C with the exception of having identical sequences to target (i.e., mismatched) at the indicated positions (FIG. 13) counted from the 5′ end of the guide sequence, and double-stranded plasmid 1 target DNA were incubated in buffer 1 for the indicated times (minutes; FIG. 13). Lanes indicated by 0 MM (MM—mismatches)) show cleavage of plasmid 1 (5481 bp; FIG. 9A) using guides fully complementary to the target generated the expected 980 bp and 4501 bp double-stranded DNA products as well as side-products. Lanes indicated by 1-2 MM to 4-5 MM show cleavage of plasmid 1 using guides containing dinucleotide mismatches sequentially from guide position 1 to guide position 5 generated the expected 980 bp and 4501 bp double-stranded DNA products as well as side-products. Lanes indicated by 5-6 MM to 15-16 MM show cleavage of plasmid 1 using guides containing dinucleotide mismatches sequentially from guide position 5 to guide position 16 generated the expected 980 bp and 4501 bp double-stranded DNA products with a reduction in observed side-products.

The data suggest that mismatches in the recruiting region are not tolerated from guide position 1 (g1) through guide position 5 (g5). Mismatches are tolerated at the 3′ end of the recruiting region (g6-g8) through the stabilization domain (g9-g16).

### Example 15

### Cloning Using TtAG: Guide Molecule Complexes (Prophetic)

Argonaute:guide molecule complexes can be used to subclone double-stranded nucleic acid fragments. For example, fragment A (FIG. 14) can be cleaved with Argonaute:guide molecule complexes, wherein the guide molecules are complementary to sequences surrounding fragment A; in this example (FIG. 14), pairs of Argonaute:guide molecule complexes are necessary so that both the 5′ and 3′ strands are cleaved at each end of fragment A, and as shown, can be designed to create 3′ and 5′ overhangs (“sticky-ends”; FIG. 14). This cleavage step liberates fragment A from plasmid 1 (FIG. 14), and has desirable sticky-ends that can be used to facilitate directional cloning. Plasmid 2, which is to receive fragment A, is treated with Argonaute:guide molecule complexes to liberate fragment B, leaving the plasmid 2 backbone with complementary sticky ends to those of liberated fragment A (FIG. 14). Digested fragment A and plasmid 2 backbone are isolated, combined with ligase under appropriate conditions, to generate a plasmid 2 with fragment A replacing previous fragment B. In a variation of this approach, the guide molecules are designed such as to create longer sticky ends, about 18-24 (or more) nucleotides in length. When the prepared fragments are mixed and incubated at an appropriate temperature, the complementary sticky ends anneal and are sufficiently stable to maintain the duplex. Ligase is not needed to complete the subcloning, and the molecule is transformed directly into a host cell, or used in another application.

In an example where a desired fragment (FIG. 15, “dsDNA2”) is to be cloned into another double stranded DNA molecule (FIG. 15, “dsDNA1”), but where complementary 3′ and 5′ overhangs cannot be generated, pairs of Argonaute:guide molecule complexes are used to generate fragment dsDNA2 with 3′ and 5′ overhangs. dsDNA1 (the DNA into which the dsDNA1 fragment is to be subcloned) is prepared with different pairs of Argonaute:guide molecule complexes using preferably 12-15 nucleotide guides and buffer 1 or 2 to create a DNA backbone that also has 3′ and 5′ overhangs. In this case, however, the 3′ and 5′ overhangs of dsDNA2 are not complementary to the 5′ and 3′ overhangs of the dsDNA1. To complete the cloning, “bridge” oligonucleotides (FIG. 15, “bridge oligo 1” and “bridge oligo 2”) that are complementary to overhangs of both the dsDNA1 and dsDNA2 (FIG. 15) are hybridzed, and the complex incubated with polymerase and ligase under appropriate conditions to subclone fragment dsDNA2 into the backbone of dsDNA1 (FIG. 15, bottom).

### Example 16

### Argonaute: Guide Molecule Complexes as Probes

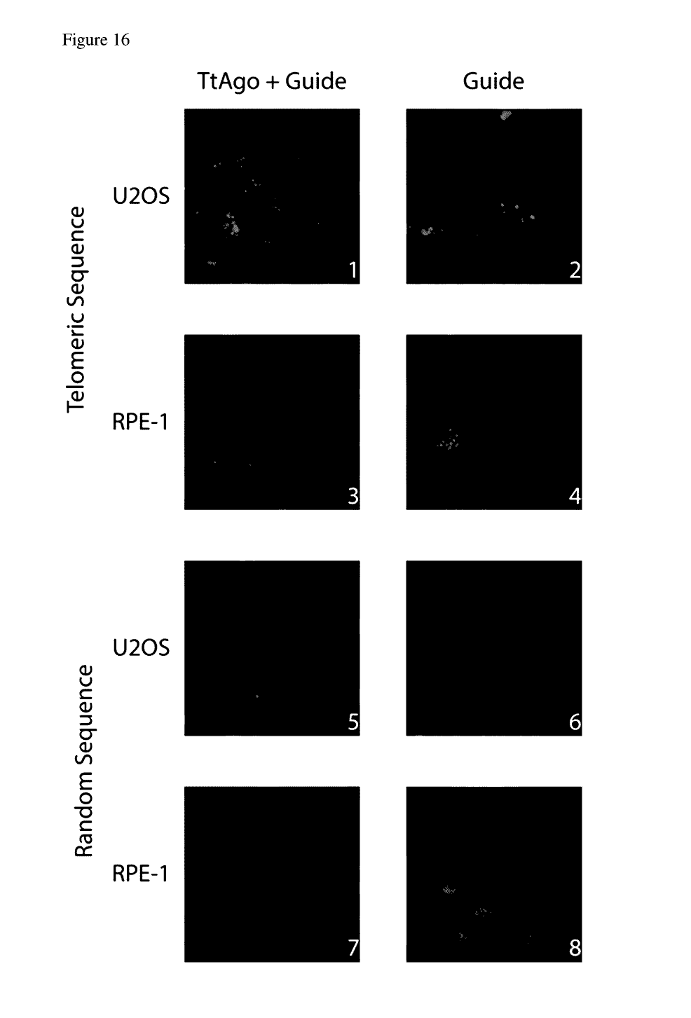

For purposes of exploring TtAgo-guide complex as a probe, we used fixed human osteosarcoma (U2OS) and retina pigmented epithelial (RPE-1) cells. U2OS cells contain elongated telomeric repeats at the ends of their chromosomes compared to normal cells, and they have been used for demonstrating ability of imaging techniques whereas RPE-1 cells have shorter telomeric repeats (Ma et al., 2013, Visualization of repetitive DNA sequences in human chromosomes with transcription-activator like effectors. Proc. Natl. Acad. Sci. USA, 110(52), 21,048-21,053). These telomeric repeats contain displacement loops (D-loops) that possess portions of single-strandedness (Griffith et al., 1999, Mammalian telomeres end in a large duplex loop. Cell, 97(4), 503-514) that can serve as targets for TtAgo-guide complexes. In FIG. 16 we used U2OS and retina pigmented epithelial (RPE-1) cells to demonstrate the specificity of TtAgo-guide complexes acting as probes.

Sixteen (16) nucleotide telomeric or random DNA guide sequences either in complex with TtAgo or alone were incubated for 30 minutes with fixed U2OS at guide concentration of 1 nM or fixed RPE-1 cells at guide concentration of 10 nM. Nuclear staining was performed with DAPI and images were acquired according to the above method. The results are shown in FIG. 16. FIG. 16.1 shows probing with TtAgo in complex with the telomeric DNA guide which exhibited many nuclear punctate signals in U2OS cells. FIG. 16.2 shows probing with telomeric DNA guide alone which exhibited minimal nuclear staining in U2OS cells. FIG. 16.3 shows probing with TtAgo in complex with the telomeric DNA guide which demonstrated reduced nuclear binding in RPE-1 cells. FIG. 16.4 shows probing with telomeric DNA guide alone which exhibited reduced nuclear staining in RPE-1 cells. FIG. 16.5 shows probing with TtAgo in complex with the random DNA guide which showed no nuclear staining in U2OS cells. FIG. 16.6 shows probing with a random DNA guide alone, which exhibited no nuclear staining in U2OS cells. FIG. 16.7 shows probing with TtAgo in complex with the random DNA guide which demonstrated no nuclear binding in RPE-1 cells. FIG. 16.8 shows probing with random DNA guide alone which exhibited no nuclear staining in RPE-1 cells.

The larger number of punctate, nuclear signals observed with TtAgo-guide complex with the telomeric sequence in U2OS cells (FIG. 16.1) compared to the guide alone (FIG. 16.2) demonstrates the ability of TtAgo to impart enhanced binding properties upon the guide molecule. The similar signal intensity and localization of the complex (FIG. 16.3) compared to the telomeric guide alone (FIG. 16.4) in cells lacking increased telomeric repeats demonstrates the level of minimal telomeric binding possible with this sequence. The lack of binding of TtAgo-guide complex (FIG. 16.5, FIG. 16.7) or guide alone (FIG. 16.6, FIG. 16.8) with the random guide sequence demonstrates the specificity of the telomeric guides, in complex or alone, for their targets.

### Example 17

### Experimental Procedures

### Example 17.1

### Argonaute Purification

Expression and purification of TtAgo was as described (Wang et al., 2008. Structure of the guide-strand-containing Argonaute silencing complex. Nature, 456 (7219), 209-213) except for the final chromatography step. Briefly, TtAgo was amplified from genomic DNA and cloned into pET SUMO (Life Technologies (Fisher Scientific); Waltham, Mass.). Expression in E. coli BL21-DE3 was induced with 0.1 mM isopropyl-β-D-thiogalactoside at 37° C. for 4 h. Cells were lysed with a micro-fluidizer (Microfluidics; Westwood, Mass.) and TtAgo isolated by HisTrap HP (GE Healthcare; Marlborough, Mass.) chromatography. The amino-terminal six-histidine tag was cleaved from TtAgo using SUMO-protease (Life Technologies), dialyzed into 20 mM Tris-HCl pH 7.5, 0.5 M NaCl, 2 mM MgCl₂, 10% glycerol, 2 mM DTT and passed through a HisTrap HP (GE Healthcare) column. After dialysis into 18 mM HEPES-KOH, pH 7.4, 100 mM potassium acetate, 3 mM magnesium acetate, 0.1 mM EDTA, 0.01% (w/v) IGEPAL CA-630, 5 mM dithiothreitol, 10% (w/v) glycerol), the protein was further purified by HiTrap SP HP (GE Healthcare) chromatography. Finally, purified TtAgo was dialyzed into storage buffer (18 mM HEPES-KOH, pH 7.4, 250 mM potassium acetate, 3 mM magnesium acetate, 0.1 mM EDTA, 0.01% (w/v) IGEPAL CA-630, 5 mM dithiothreitol, 20% (w/v) glycerol).

Mouse AGO2-RISC was assembled using an siRNA bearing a 3′ Alexa Fluor 555 dye (Life Technologies, Grand Island, N.Y.) on its guide strand in S100 extract made from Ago2−/− mouse embryonic fibroblasts over-expressing mouse AGO2 (O'Carroll et al., Genes Dev 21, 1999-2004, 2007) and then purified as described (Flores-Jasso et al., RNA 19, 271-279, 2013). TtAgo, cloned from genomic DNA into Champion pET SUMO (Life Technologies) was expressed in E. coli BL21-DE3, then purified and assembled with a 16 nucleotide DNA guide strand containing a 3′ Alexa Fluor 555 dye. Free DNA guide strand was removed using a Q Sepharose Fast Flow spin column (GE Healthcare Bio-Sciences, Piscataway, N.J.). Active AGO2 concentration was determined by pre-steady state kinetics (Wee et al., Cell 151, 1055-1067, 2012); TtAgo concentration was determined by guide strand fluorescence.

### Example 17.2

### RNA and DNA Substrates

RNA substrates were prepared by in vitro transcription as described (Haley et al., Methods 30, 330-336, 2003) except 5′-biotin-GMP (TriLink Biotechnologies, San Diego, Calif.) was used at a 4:1 excess over GTP. RNA was gel purified, and a DNA extension containing 17 Alexa Fluor 647-aha-dUTPs (Life Technologies) was added by DNA-templated 3′ end extension using Klenow. For 3′-tethered targets, we used a DNA template to generate a DNA piece (187 nucleotide) containing 17 Alexa Fluor 647-aha-dUTP moieties and then ligated a synthetic, 45 nucleotide DNA/RNA linker to its 3′ end with T4 DNA ligase and a DNA splint. The DNA containing piece with the 3′ ligated DNA/RNA linker (15 nucleotide DNA, 30 nucleotide RNA) was ligated onto the 5′ end of the in vitro transcribed RNA by splinted ligation as described (Moore and Query, Methods Enzymol 317, 109-123, 2000). All DNA substrates were chemically synthesized (Integrated DNA Technologies, Coralville, Iowa) and a 148 nucleotide DNA extension with (17) Alexa Fluor 647-aha-dUTPs was added by DNA-templated 3″ end Klenow extension. Table 51 contains all substrate, linker, and template sequences.

### Example 17.3

### Single—Molecule Spectroscopy and Data Analysis

Imaging was at 37° C. on an IX81-ZDC2 zero-drift, inverted microscope (Olympus, Tokyo, Japan) equipped with a motorized, multicolor TIRF illuminator, 100 W lasers, and a 100× high numerical aperture objective. Images were recorded with two EM-CCD cameras (ImagEM, Hamamatsu Photonics, Hamamatsu, Japan) using a dichroic image splitter (DC2, Photometrics, Tucson, Ariz.) to separate fluorescent emission from the two spectrally distinct fluorescent dyes. All acquisition parameters were controlled with Metamorph software (Molecular Devices, Sunnyvale, Calif.), and image analysis was performed in MATLAB using custom scripts and a co-localization analysis package developed by the Gelles laboratory (L. Friedman and J. Gelles, unpublished). Location of Alexa647 target molecules and mapping of target locations to the Alexa555 channel were as described (Crocker and Grier, Journal of Colloid and Interface Science 179, 298-310, 1996; Friedman et al., Biophys J 91, 1023-1031, 2013). All on-rate and off-rate measurements were corrected for non-specific binding to the slide surface; all dissociation rates were corrected for photobleaching. Global fitting of kinetic curves to a unified kinetic scheme was performed numerically in DynaFit 4 (BioKin, Watertown, Mass.) (Kuzmic, Anal Biochem 237, 260-273, 1996).

### Example 17.4

### Argonaute-Mediated Cleavage of Nucleic Acids at Designer Sites

Purified TtAgo was programmed with synthetic, single-stranded DNA guides containing a 5′-phosphate by incubation in 18 mM HEPES-KOH, pH 7.5 at 22° C., 50 mM sodium chloride, 3 mM MnCl₂, 0.01% IGEPAL CA-630 (w/v), 5 mM dithiothreitol, 10% (w/v) glycerol (hereafter described as buffer 1), at 75° C. for 30 min. Protein was used at a three-fold molar excess to guides. The reaction was then cooled to room temperature and gently mixed. After centrifuging briefly, the DNA to be cleaved was added (3:1:1 Argonaute:guide:target) and incubation at 75° C. resumed. The resulting DNA fragments were analyzed by agarose gel electrophoresis.

An alternative cleavage assay method was employed that is identical to the above cleavage method except for the substitution of buffer 1 for one comprised of 18 mM HEPES-KOH, pH 7.5 at 22° C., 75 mM mono-sodium glutamate, 3 mM MnCl₂, 0.01% (w/v) IGEPAL CA-630, 5 mM dithiothreitol, 10% (w/v) glycerol which is hereafter referred to as buffer 2.

### Example 17.5

### Methods for Argonaute:Guide Complex as Imaging Probes

3′ Fluorescent Labeling of DNA Guide Oligonucleotides

To 25 μL of 0.1 mM sodium tetraborate (Na₂B₄O₇) pH 8.5 was added 5 μL 1 mM 16 nt single-stranded DNA oligonucleotide with 5′ phosphate and 3′-amino modifications and 3 μL 20 mM Alexa-647 in DMSO. This was incubated at 4° C. for 16 hours protected from light. This was purified on a denaturing urea polyacrylamide gel, the band excised and eluted for 16 hours into 0.3 M NaCl. The labeled nucleotide was isolated by ethanol precipitation and quantified by fluorescence using a spectrophotometer (Beckman Coulter, Inc.).

TtAgo-Guide Complex Purification

TtAgo-guide complex was assembled by incubating 3 μM 16 nt, synthetic, single-stranded DNA oligonucleotide containing a 5′ phosphate and a 3′ Alexa647 dye (Invitrogen) with 1 μM TtAgo for 30 min at 75° C. in a reaction buffer comprising 18 mM HEPES-KOH, pH 7.4, 350 mM potassium acetate, 3 mM magnesium acetate, 0.01% (w/v) IGEPAL CA-630, 5 mM dithiothreitol, 10% (w/v) glycerol. Unassembled DNA guide was removed by passing the reaction through a Q Sepharose Fast Flow (GE Healthcare) spin column. The concentration of RISC was determined by guide fluorescence using a Typhoon FLA-7000 (GE Healthcare).

Cell Culture

Human osteosarcoma U2OS cells were cultured at 37° C. in Dulbecco-modified Eagle's Minimum Essential Medium (DMEM; Life Technologies) supplemented with heat inactivated 10% (v/v) FBS and penicillin (5000 U) and streptomycin (500 μg). Human RPE-1 cells were cultured at 37° C. in DMEM/F12 medium supplemented with heat inactivated 10% (v/v) FBS and penicillin (5000 U) and streptomycin (500 μg).

Cell Fixation

Human osteosarcoma (U2OS) cells and retinal pigment epithelial (RPE-1) cells were grown to confluence on 2-cm No. 1.5 round cover slips (Electron Microscopy Services) in 12-well plates in DMEM (U2OS) or DMEM/F12 (RPE-1) media. The media was aspirated and cells were washed once with 1× phosphate-buffered saline (PBS). PBS was aspirated, and cells were fixed by the addition of a mixture of 90% methanol and 10% 1×PBS pre-chilled at −20° C., followed by incubation at −20° C. for 10 minutes. Methanol-PBS mixture was aspirated, and the cells were washed 3 times with 1×PBS.

Fixed Cell Imaging

20-microliter drops of 0.1-10 nM TtAgo or free DNA guide strands in PBS were placed on a piece of Parafilm. Cover slips with fixed cells were removed from the 12-well plate and placed cell side down on the drops, ensuring even contact of solution with the entire surface of the slide. After 30 minutes of incubation protected from light, the cover slips were placed in a 12-well plate (cell side up), and washed with 3 changes of PBS. Cover slips were wick dried on a paper towel. Each cover slip was then pressed cell side down onto a drop of ProLong® Gold Antifade Mountant with DAPI (Life Technologies) that was placed in the middle of a rectangular microscope slide. Slides were flipped onto paper towel and allowed to dry for 10 minutes while protected from light. Slides were imaged on Olympus IX-81 inverted microscope with a 100× N.A. 1.49 objective using 405 and 640 nm laser excitation for DAPI and Alexa647-labeled guide strands, respectively.

All cited references are herein incorporated by reference in their entireties.

1. A method of cleaving an RNA or DNA molecule, comprising binding to a target RNA or DNA sequence a complex comprising an Argonaute polypeptide and a heterologous, single-stranded oligonucleotide guide molecule that comprises a recruiting domain comprising at least 8 nucleotides at the 5′ end of the guide molecule (g1-g8) and a stabilization domain adjacent and 3′ to the recruiting domain and comprising at least 4 nucleotides (g9-g12) in a sample, wherein the stabilization domain of the guide molecule has sufficient complementarity to its target RNA or DNA sequence such that the Argonaute polypeptide:guide molecule complex binds stably to the target RNA or DNA sequence, and allowing the Argonaute polypeptide:guide molecule to cleave the RNA or DNA molecule.

2. The method of claim 1, wherein the stabilization domain consists of 4 to 8 nucleotides.

3. The method of claim 1, wherein the recruiting domain consists of 8 nucleotides, and the stabilization domain consists of 4 to 8 nucleotides.

4. The method of claim 1, wherein the oligonucleotide guide molecule is a DNA guide molecule.

5. The method of claim 1, wherein the target RNA or DNA is single-stranded or double-stranded.

6.-7. (canceled)

8. The method of claim 1, wherein the guide molecule comprises one or more mismatches 3′ of g5.

9. (canceled)

10. The method of claim 8, wherein the guide molecule comprises two mismatches 3′ of g5 and 5′ of g9.

11. The method of claim 8, wherein the guide molecule comprises two mismatches 3′ of g8 to the 3′ end of the molecule.

12.-16. (canceled)

17. The method of claim 1, wherein the sample comprises a solution comprising a salt.

18.-22. (canceled)

23. The method of claim 17, wherein the solution further comprises a buffer.

24. The method of claim 23, wherein the buffer is selected from the group consisting of N-(2-acetamido)-2-aminoethanesulfonic acid (ACES), N-(2-acetamido)iminodiacetic acid (ADA), N,N-bis(2-hydroxyethyl)-2-aminoethanesulfonic acid (BES), 2-(N-morpholino)ethanesulfonic acid (MES), 3-(N-morpholino)-propanesulfonic acid (MOPS), 3-(N-morpholinyl)-2-hydroxypropanesulfonic acid (MOPSO), piperazine-N,N′-bis(2-ethanesulfonic acid) [Pipes], N-tris-(hyrdroxymethyl)-methyl-2-aminoethanesulfonic acid (TES), 3-[N-tris (hydroxymethyl) methylamino]-2-hydroxypropanesulfonic acid (TAPSO), and 3-[N-tris-(hydroxymethyl-mettlylamino]-propanesulfonic acid (TAPS); and 2-[4-(2-hydroxyethyl)piperazin-1-yl]ethanesulfonic acid (HEPES).

25.-28. (canceled)

29. The method of claim 24, wherein the pH of the buffer is from about 7 to about 8.8.

30. (canceled)

31. The method of claim 17, wherein the solution further comprises a reducing agent.

32. The method of claim 31, wherein the reducing agent comprises dithiothreitol (DTT) or 2-mercaptoethanol (β-mercaptoethanol).

33.-34. (canceled)

35. The method of claim 17, wherein the solution further comprises a detergent.

36. The method of claim 35, wherein the detergent is a nonionic, non-denaturing detergent or a zwitterionic nondenaturing detergent.

37.-40. (canceled)

41. The method of claim 17, wherein the solution further comprises glycerol or a sugar.

42.-43. (canceled)

44. The method of claim 17, wherein the solution further comprises a divalent metal cation.

45.-47. (canceled)

48. The method of claim 17, wherein the solution comprises (1) 18 mM HEPES-KOH, pH 7.4; 50 mM NaCl, 3 mM MnCl₂, 0.01% octylphenoxy poly(ethyleneoxy)ethanol, 5 mM DTT, and 10% glycerol or (2) 18 mM HEPES-KOH, pH 7.4; 75 mM C₅H₈NNaO₄, 3 mM MnCl₂, 0.01% octylphenoxy poly(ethyleneoxy)ethanol, 5 mM DTT, and 10% glycerol.

49.-52. (canceled)

53. A method of subcloning a desired double stranded nucleic acid fragment from a donor double stranded nucleic acid molecule (donor fragment) to an acceptor double stranded nucleic acid molecule (acceptor molecule), comprising the steps of:
(a) cleaving the desired donor fragment according to the method of claim 1, wherein
(i) a first Argonaute:guide molecule complex targets a first region of a first strand of the donor fragment;
(ii) a second Argonaute guide molecule complex that targets the first region of a second strand of the donor fragment, such that the targeted region of the first strand and the targeted region of the second strand partially overlap such that cleavage by the first and second Argonaute:guide molecule complex creates a first sticky end;
(iii) a third Argonaute:guide molecule complex that targets a second region of the first strand of the donor fragment;
(iv) a fourth Argonaute:guide molecule complex targets the second region of the second strand of the donor fragment as that of the third Argonaute:guide molecule complex, such that the targeted region of the first strand of the target nucleic acid and the targeted region of the second strand of target nucleic acid partially overlap such that cleavage by the third and fourth Argonaute:guide molecule complex creates a second sticky end;
(v) isolating the cleaved desired fragment;
(b) cleaving the acceptor molecule according to the method of claim 1, wherein
(i) steps (a)(i)-(a)(iv) are repeated for the acceptor molecule, thus creating third and fourth sticky ends that are complementary to the first and second sticky ends;
(ii) isolating the cleaved acceptor molecule; and
(c) combining the molecules from steps (a) and (b) to create a mixture and incubating the mixture under appropriate conditions to form a new molecule comprising the desired donor fragment subcloned into the acceptor molecule.

54. The method of claim 53, wherein ligase is added to the mixture of step (c).

55. The method of claim 53, wherein the sticky ends are from about 18 to 24 nucleotides long, and ligase is not added to the mixture of step (c).

56. The method of claim 53, wherein the sticky ends are not complementary, and further comprising in step (c) combining a first single-stranded oligonucleotide that is complementary to a sticky end of the desired fragment and to a sticky end of the acceptor molecule such that the oligonucleotide bridges the sticky ends, and a second single-stranded oligonucleotide that is complementary to the other sticky ends of the desired fragment and of the acceptor molecule, such that the oligonucleotide bridges the sticky ends; and treating the mixture with polymerase and ligase.

57. A kit, comprising an Argonaute polypeptide and a single-stranded oligonucleotide guide molecule that comprises a recruiting domain comprising 8 nucleotides at the 5′ end of the guide molecule (g1-g8) and a stabilization domain adjacent and 3′ to the recruiting domain and comprising at least 4 nucleotides (g9-g12) and having a sequence sufficiently complementary to a target RNA or DNA molecule nucleic acid sequence such that the Argonaute polypeptide:guide molecule complex binds stably to the target RNA or DNA sequence.

58. The kit of claim 57, wherein the oligonucleotide guide molecule is a DNA guide molecule.

59. The kit of claim 57, further comprising a buffer.

60. (canceled)

61. The kit of claim 59, wherein the buffer further comprises at least one selected from the group consisting of: a salt, a detergent, a reducing agent, a divalent metal cation, glycerol and sugar.

62. The kit of claim 61, wherein the buffer is selected from the group consisting of N-(2-acetamido)-2-aminoethanesulfonic acid (ACES), N-(2-acetamido)iminodiacetic acid (ADA), N,N-bis(2-hydroxyethyl)-2-aminoethanesulfonic acid (BES), 2-(N-morpholino)ethanesulfonic acid (MES), 3-(N-morpholino)-propanesulfonic acid (MOPS), 3-(N-morpholinyl)-2-hydroxypropanesulfonic acid (MOPSO), piperazine-N,N′-bis(2-ethanesulfonic acid) [Pipes], N-tris-(hyrdroxymethyl)-methyl-2-aminoethanesulfonic acid (TES), 3-[N-tris (hydroxymethyl) methylamino]-2-hydroxypropanesulfonic acid (TAPSO), and 3-[N-tris-(hydroxymethyl-mettlylamino]-propanesulfonic acid (TAPS); and 2-[4-(2-hydroxyethyl)piperazin-1-yl]ethanesulfonic acid (HEPES).

63. The kit of claim 61, wherein the salt is NaCl, KCl, or C₅H₈NNaO₄.

64. The kit of claim 61, wherein the detergent is a nonionic non-denaturing detergent or a nondenaturing zwitterionic detergent.

65. (canceled)

66. The kit of claim 59, wherein the buffer comprises (1) 18 mM HEPES-KOH, pH 7.4; 50 mM NaCl, 3 mM MnCl2, 0.01% octylphenoxy poly(ethyleneoxy)ethanol, 5 mM DTT, and 10% glycerol, or (2) 18 mM HEPES-KOH, pH 7.4; 75 mM C₅H₈NNaO₄, 3 mM MnCl₂, 0.01% octylphenoxy poly(ethyleneoxy)ethanol, 5 mM DTT, and 10% glycerol.

67. (canceled)

68. The kit of claim 57, wherein the guide molecule binds a disease marker sequence, a disorder marker sequence, or an infectious agent sequence.

69. The kit of claim 57, wherein the guide molecule further comprises a detectable label.

70.-71. (canceled)

72. The kit of claim 57, further comprising a probe to detect the target RNA or DNA sequence.

73. The kit of claim 69, wherein the detectable label is at least one fluorophore, the fluorophore localized to the recruiting or stabilization domain forming a first arm, wherein
the guide molecule comprises additional sequence at the 3′ end that is complementary to the domain comprising the at least one fluorophore, the sequence labeled with at least one quencher and forming a second arm;
the first arm separated from the second arm by not more than about 60 nucleotides;
the guide molecule forming with the target RNA or DNA sequence under detection conditions a double-stranded hybrid having a first strength;
the first and second arm sequences having sufficient complementarity to one another to form under detection conditions a double-stranded stem hybrid having a second strength less than the first strength, whereby in the absence of the target RNA or DNA sequence fluorescence of the at least one fluorophore is quenched; and
the first and second hybrid strengths being selected such that the guide molecule fluoresces when the at least one fluorophore is stimulated under detection conditions in the presence of the target RNA or DNA sequence.

74.-81. (canceled)

82. A method of recruiting an Argonaute polypeptide to a heterologous target RNA or DNA sequence comprising combining the Argonaute polypeptide with a heterologous, single-stranded oligonucleotide guide molecule that comprises a recruiting domain of at least 8 nucleotides at its 5′ end of the guide molecule (g1-g8) and a stabilization domain adjacent and 3′ to the recruiting domain and comprising at least 4 nucleotides (g9-g12), wherein the stabilization domain of the guide molecule has sufficient complementarity to the target RNA or DNA sequence such that the Argonaute polypeptide:guide molecule complex stably binds to the target RNA or DNA sequence.

83. The method of claim 82, wherein the stabilization domain consists of 4 to 8 nucleotides.

84. The method of claim 82, wherein the recruiting domain consists of 8 nucleotides, and the stabilization domain consists of 4 to 8 nucleotides.

85. The method of claim 82, wherein the oligonucleotide guide molecule is a DNA guide molecule.

86. The method of claim 82, wherein the target RNA or DNA is single-stranded or double-stranded.

87.-88. (canceled)

89. The method of claim 82, wherein the guide molecule comprises one or more mismatches 3′ of g5.

90. (canceled)

91. The method of claim 89, wherein the guide molecule comprises two mismatches 3′ of g5 and 5′ of g9.

92. The method of claim 89, wherein the guide molecule comprises two mismatches 3′ of g8 to the 3′ end of the molecule.

93.-99. (canceled)

100. The method of claim 82, wherein the heterologous target RNA or DNA is a eukaryotic, prokaryotic, or viral mRNA or gene.

101. The method of claim 82, wherein the Argonaute polypeptide:guide molecule complex is attached to a solid support.

102. The method of claim 101, wherein contacting the Argonaute polypeptide:guide molecule complex to a sample depletes the sample of the target RNA or DNA molecule.

103. The method of claim 102, wherein the depleted sequence is an rRNA sequence.

104. (canceled)

105. The method of claim 82, wherein the target comprises an RNA molecule, and the guide molecule targets a splice site on the mRNA molecule.

106.-109. (canceled)

110. The method of claim 82, further comprising detecting the Argonaute polypeptide:guide molecule complex.

111. The method of claim 110, wherein the guide molecule further comprises a detectable label.

112.-113. (canceled)

114. The method of claim 111, wherein the detecting comprises detecting the target RNA or DNA nucleotide sequence.

115. The method of claim 114, wherein detecting the target RNA or DNA nucleotide sequence comprising using a probe to detect the target RNA or DNA nucleotide sequence.

116. The method of claim 111, wherein the detectable label is at least one fluorophore, the fluorophore localized to the recruiting or stabilization domain forming a first arm, wherein
the guide molecule comprises additional sequence at the 3′ end that is complementary to the domain comprising at least one fluorophore, the sequence labeled with at least one quencher and forming a second arm;
the first arm separated from the second arm by not more than about 60 nucleotides;
the guide molecule forming with the target RNA or DNA sequence under detection conditions a double-stranded hybrid having a first strength;
the first and second arm sequences having sufficient complementarity to one another to form under detection conditions a double-stranded stem hybrid having a second strength less than the first strength, whereby in the absence of the target RNA or DNA sequence fluorescence of the at least one fluorophore is quenched; and
the first and second hybrid strengths being selected such that the guide molecule fluoresces when the at least one fluorophore is stimulated under detection conditions in the presence of the target RNA or DNA sequence.

117. The method of claim 110, wherein the guide molecule further comprises an additional sequence added to the 3′ end of the guide molecule.

118. The method of claim 117, wherein the detecting is detecting the additional sequence of the guide molecule.

119.-123. (canceled)

124. The method of claim 110, wherein the target RNA or DNA molecule encodes a disease marker sequence, a disorder marker sequence, or an infectious agent sequence.

125. A method of identifying an RNA binding polypeptide comprising binding to a target RNA sequence in an RNA molecule a complex comprising an Argonaute polypeptide and a heterologous, single-stranded oligonucleotide guide molecule that comprises a recruiting domain comprising at least 8 nucleotides at its 5′ end of the guide molecule (g1-g8) and a stabilization domain adjacent and 3′ to the recruiting domain and comprising at least 4 nucleotides (g9-g12) in a sample, wherein the stabilization domain of the guide molecule has sufficient complementarity to the target RNA sequence such that the Argonaute polypeptide:guide molecule complex binds stably to the target RNA sequence, isolating the Argonaute polypeptide:guide molecule complex bound to the target RNA sequence, and detecting polypeptides bound to the RNA molecule comprising the target RNA sequence.

126. The method of claim 125, wherein the stabilization domain consists of 4 to 8 nucleotides.

127. The method of claim 125, wherein the recruiting domain consists of 8 nucleotides, and the stabilization domain consists of 4 to 8 nucleotides.

128. The method of claim 125, wherein the oligonucleotide guide molecule is a DNA guide molecule.

129. The method of claim 125, wherein the target RNA is single-stranded or double-stranded.

130.-131. (canceled)

132. The method of claim 125, wherein the guide molecule comprises one or more mismatches 3′ of g5.

133. (canceled)

134. The method of claim 132, wherein the guide molecule comprises two mismatches 3′ of g5 and 5′ of g9.

135. The method of claim 132, wherein the guide molecule comprises two mismatches 3′ of g8 to the 3′ end of the molecule.

136.-146. (canceled)
